(19) United States
(12) Patent Application Publication
(10) Pub. No.: US 2025/0084432 A1
(43) Pub. Date: Mar. 13, 2025

(54) LODGING RESISTANCE IN ERAGROSTIS TEF

(71) Applicant: Donald Danforth Plant Science Center, St. Louis, MO (US)

(72) Inventors: Getu Beyene Duguma, St. Louis, MO (US); Donald J. Mackenzie, St. Louis, MO (US); Justin Villmer, St. Louis, MO (US)

(21) Appl. No.: 18/720,375

(22) PCT Filed: Dec. 16, 2022
(86) PCT No.: PCT/US2022/081856

§ 371 Date: Jun. 14, 2024

Related U.S. Application Data
(60) Provisional application No. 63/290,486, filed on Dec. 16, 2021.

(51) Int. Cl. C12N 15/82; A01H 6/46; C12N 9/22; C12N 15/11

(52) U.S. Cl. CPC C12N 15/8298; A01H 6/46; C12N 9/22; C12N 15/111; C12N 15/8294; C12N 15/8297; C12N 2310/20

(57) ABSTRACT

Genetically modified lodging resistant Eragrostis plants and methods of producing lodging resistance in an Eragrostis plant, part thereof, or plant cell thereof are disclosed. Methods of improving the yield of an Eragrostis plant are also disclosed.

### CROSS-REFERENCE TO RELATED APPLICATIONS

This application claims priority from Provisional Application No. 63/290,486, filed Dec. 16, 2021, the entire contents of which are hereby incorporated by reference.

### SEQUENCE LISTING

This application contains a Sequence Listing that has been submitted in .XML format via PatentCenter and is hereby incorporated by reference in its entirety. The XMI is named 077875-747032, and is 98 kilobytes in size.

### FIELD OF THE INVENTION

The present disclosure provides genetically modified lodging resistant Eragrostis plants.

### BACKGROUND OF THE INVENTION

Plants with short stature have had a major impact on world agriculture. This notion is best exemplified by the success of the green revolution, which was made possible by the advent of dwarf varieties of rice and wheat. Agronomic interest in short plants derives largely from their ability to resist lodging caused by wind, rain, or higher densities, which allows them to effectively convert increased fertilizer input into higher yields.

Teff (Eragrostis tef) is an ancient grain cultivated in Ethiopia as a major staple food and cash crop for millions and a valued forage crop. Teff is tolerant to pests and diseases and suffers minimal postharvest losses. Teff can grow relatively well in extremes of conditions not suitable for other cereals including wider range of altitudes (up to 3000 meters above sea level) and moisture extremes, consequently rendering this grass more climate resilient than most crops. Teff grain is also valued as a high protein source, with a balanced and complete set of amino acids, and is a rich source of minerals including iron and calcium and is gluten-free which contributes to food products suitable for people with celiac disease.

Over 50 years of research and development between 1960-2012 have brought incremental improvements in teff grain yield with genetic gains of 0.5 to 0.79 percent per year. However, teff yield remains very low compared to the other major cereals, averaging about 1.7 t/ha. Lodging, competition from weeds, grain shattering, and low productivity of landraces (the types predominantly cultivated by farmers in Ethiopia) are major limitations to teff production. Lodging suppresses yield on average by 17 percent and substantially reduces the quality of harvested grain and straw. Currently, all improved varieties of teff are susceptible to lodging. Decades of conventional breeding approaches to improve lodging resistance in teff has been of limited success.

Accordingly, there is a need for lodging resistant teff plants.

### SUMMARY OF THE INVENTION

One aspect of the instant disclosure encompasses a genetically modified lodging resistant Eragrostis plant, part thereof, or plant cell thereof. The plant comprises two or more nucleic acid modifications that result in a reduction in two or more hormone activities selected from auxin transport activity, gibberellic acid (GA) biosynthesis activity, and brassinosteroid (BR) signaling activity. Lodging resistance negatively correlates with plant height, and the two or more nucleic acid modifications result in a synergistic reduction in height of the Eragrostis plant.

The nucleic acid modification that results in a reduced GA biosynthesis activity can be a nucleic acid modification in a nucleic acid sequence encoding an SD1 protein, the nucleic acid modification that results in a reduced BR signaling activity can be a nucleic acid modification in a nucleic acid sequence encoding a DW1 protein, and the nucleic acid modification that results in a reduced auxin transport activity can be a nucleic acid modification in a nucleic acid sequence encoding a DW3 protein. In some aspects, the plant comprises two or more nucleic acid modifications that result in a reduction of auxin transport activity and BR signaling activity. In other aspects, the plant comprises three or more nucleic acid modifications that result in a reduction of auxin transport activity, GA biosynthesis activity, and BR signaling activity.

In some aspects, the genetically modified lodging resistant Eragrostis plant is Eragrostis tef. When the genetically modified plant, part thereof, or plant cell thereof, is Eragrostis tef, the lodging resistant genetically modified plant the synergistic reduction in height comprises an approximate 50 percent reduction in height of the plant when compared to a wild type plant.

When the genetically modified plant, part thereof, or plant cell thereof, is Eragrostis tef, the nucleic acid modification that results in a reduction in GA biosynthesis can be a nucleic acid modification in a nucleic acid sequence encoding an SD1A protein and a nucleic acid modification in a nucleic acid sequence encoding an SD1B protein. In some aspects, the SD1A protein comprises an amino acid sequence comprising at least about 75% or more, at least about 85% or more, at least about 95% or more, or 100% sequence identity with an amino acid sequence of SEQ ID NO: 17; and the SD1B protein comprises an amino acid sequence comprising at least about 75% or more, at least about 85% or more, at least about 95% or more, or 100% sequence identity with an amino acid sequence of SEQ ID NO: 19, or a combination thereof. In some aspects, the SD1A protein is encoded by a nucleic acid sequence comprising at least about 75% or more, at least about 85% or more, at least about 95% or more, or 100% sequence identity with a nucleic acid sequence of SEQ ID NO: 18, and the SD1B protein is encoded by a nucleic acid sequence comprising at least about 75% or more, at least about 85% or more, at least about 95% or more, or 100% sequence identity with a nucleic acid sequence of SEQ ID NO: 20. In other aspects, the nucleic acid modification in the nucleic acid sequence encoding an SD1A protein comprises at least about 75% or more, at least about 85% or more, at least about 95% or more, or 100% sequence identity with a nucleic acid sequence of SEQ ID NO: 21, and the nucleic acid modification in the nucleic acid sequence encoding an SD1B protein comprises at least about 75% or more, at least about 85% or more, at least about 95% or more, or 100% sequence identity with a nucleic acid sequence of SEQ ID NO: 22.

When the genetically modified plant, part thereof, or plant cell thereof, is Eragrostis tef, the nucleic acid modification that results in a reduction in BR signaling activity can be a nucleic acid modification in a nucleic acid sequence encoding a DW1A protein and a nucleic acid modification in a nucleic acid sequence encoding a DW1B protein. In some aspects, the DW1A protein comprises an amino acid sequence comprising at least about 75% or more, at least about 85% or more, at least about 95% or more, or 100% sequence identity with an amino acid sequence of SEQ ID NO: 23; and the DW1B protein comprises an amino acid sequence comprising at least about 75% or more, at least about 85% or more, at least about 95% or more, or 100% sequence identity with an amino acid sequence of SEQ ID NO: 25, or a combination thereof. In some aspects, the DW1A protein is encoded by a nucleic acid sequence comprising at least about 75% or more, at least about 85% or more, at least about 95% or more, or 100% sequence identity with a nucleic acid sequence of SEQ ID NO: 24, and the DW1B protein is encoded by a nucleic acid sequence comprising at least about 75% or more, at least about 85% or more, at least about 95% or more, or 100% sequence identity with a nucleic acid sequence of SEQ ID NO: 26. In some aspects, the nucleic acid modification in the nucleic acid sequence encoding the DW1A protein comprises at least about 75% or more, at least about 85% or more, at least about 95% or more, or 100% sequence identity with a nucleic acid sequence of SEQ ID NO: 27, and the nucleic acid modification in the nucleic acid sequence encoding an DW1B protein comprises at least about 75% or more, at least about 85% or more, at least about 95% or more, or 100% sequence identity with a nucleic acid sequence of SEQ ID NO: 28.

When the genetically modified plant, part thereof, or plant cell thereof, is Eragrostis tef, the nucleic acid modification that results in a reduction in auxin transport activity can be a nucleic acid modification in a nucleic acid sequence encoding an DW3A protein and a nucleic acid modification in a nucleic acid sequence encoding an DW3B protein. n some aspects, an amino acid sequence of the DW3A protein comprises at least about 75% or more, at least about 85% or more, at least about 95% or more, or 100% sequence identity with an amino acid sequence of SEQ ID NO: 29 and an amino acid sequence of the DW3B protein comprises at least about 75% or more, at least about 85% or more, at least about 95% or more, or 100% sequence identity with an amino acid sequence of SEQ ID NO: 31, or a combination thereof. In some aspects, the DW3A protein is encoded by a nucleic acid sequence comprising at least about 75% or more, at least about 85% or more, at least about 95% or more, or 100% sequence identity with a nucleic acid sequence of SEQ ID NO: 30, and the DW3B protein is encoded by a nucleic acid sequence comprising at least about 75% or more, at least about 85% or more, at least about 95% or more, or 100% sequence identity with a nucleic acid sequence of SEQ ID NO: 32. In some aspects, the nucleic acid modification in the nucleic acid sequence encoding the DW3A protein comprises at least about 75% or more, at least about 85% or more, at least about 95% or more, or 100% sequence identity with a nucleic acid sequence of SEQ ID NO: 33, and the nucleic acid modification in the nucleic acid sequence encoding an DW3B protein comprises at least about 75% or more, at least about 85% or more, at least about 95% or more, or 100% sequence identity with a nucleic acid sequence of SEQ ID NO: 34.

The genetically modified lodging resistant Eragrostis tef plant can comprise a nucleic acid modification in a nucleic acid sequence encoding a DW1A protein, a nucleic acid modification in a nucleic acid sequence encoding a DW1B protein, a nucleic acid modification in a nucleic acid sequence encoding a DW3A protein, and a nucleic acid modification in a nucleic acid sequence encoding a DW3B protein. In some aspects, the genetically modified lodging resistant Eragrostis tef plant comprises a nucleic acid modification in a nucleic acid sequence encoding an SD1A protein, a nucleic acid modification in a nucleic acid sequence encoding an SD1B protein, a nucleic acid modification in a nucleic acid sequence encoding a DW1A protein, a nucleic acid modification in a nucleic acid sequence encoding a DW1B protein, a nucleic acid modification in a nucleic acid sequence encoding a DW3A protein, and a nucleic acid modification in a nucleic acid sequence encoding a DW3B protein.

Another aspect of the instant disclosure encompasses an expression construct for introducing a nucleic acid modification in a nucleic acid sequence in an Eragrostis plant, part thereof, or plant cell thereof. The expression construct comprises a promoter operably linked to a polynucleotide sequence encoding a programmable nucleotide modification system targeted to a polynucleotide in a nucleic acid sequence encoding an SD1 protein, a DW1 protein, or a DW3 protein. Expression of the programmable nucleotide modification system introduces a nucleic acid modification into the nucleic acid sequence. The nucleic acid modification in the nucleic acid sequence encoding an SD1 protein results in a reduction in GA biosynthesis activity, the nucleic acid modification in the nucleic acid sequence encoding a DW1 protein results in a reduction in BR signaling activity, and the nucleic acid modification in the nucleic acid sequence encoding a DW3 protein results in a reduction in auxin transport activity. In some aspects, the expression construct further comprises a nucleic acid delivery vector comprising the nucleic acid expression construct for delivering the nucleic acid expression construct to a target cell.

The programmable nucleotide modification system can be selected from an RNA-guided clustered regularly interspersed short palindromic repeats (CRISPR)/CRISPR-associated9 (Cas) (CRISPR/Cas9) nuclease system, a CRISPR/Cpf1 nuclease system, a zinc finger nuclease (ZFN), a transcription activator-like effector nuclease (TALEN), a meganuclease, ribozyme, and a programmable DNA binding domain linked to a nuclease domain. In some aspects, the programmable nucleic acid modification system is a CRISPR/Cas system comprising a Cas9 nuclease and a guide RNAs (gRNA) comprising a sequence complementary to a target sequence within the nucleic acid sequence.

In some aspects, the Eragrostis plant, part thereof, or plant cell thereof is Eragrostis tef. When the Eragrostis plant, part thereof, or plant cell thereof is Eragrostis tef, the nucleic acid sequence can encode an SD1A protein or an SD1B protein and the gRNA comprises a nucleic acid sequence of SEQ ID NO: 35 (Sd1 gRNA1), SEQ ID NO: 36 (Sd1 gRNA2), or a combination thereof. In some aspects, the nucleic acid sequence encodes an DW1A protein or an DW1B protein and the gRNA comprises a nucleic acid sequence of SEQ ID NO: 37 (Dw1 gRNA). The nucleic acid sequence encodes an DW3A protein or an DW3B protein and the gRNA comprises a nucleic acid sequence of SEQ ID NO: 38 (Dw3 gRNA).

Yet another aspect of the instant disclosure encompasses an Eragrostis plant, part thereof, or plant cell thereof comprising one or more expression constructs for introducing a nucleic acid modification in a nucleic acid sequence in an Eragrostis plant, part thereof, or plant cell thereof. The expression constructs can be as described herein above.

One aspect of the instant disclosure encompasses a method of producing a lodging resistant Eragrostis plant, part thereof, or plant cell thereof. The method comprises introducing into the plant two or more nucleic acid modifications that result in a reduction in two or more plant hormone activities selected from auxin transport activity, gibberellic acid (GA) biosynthesis activity, and brassinosteroid (BR) signaling activity. The genetic modifications can be introduced using a programmable nucleotide modification system. The programmable nucleotide modification system can be selected from an RNA-guided clustered regularly interspersed short palindromic repeats (CRISPR)/CRISPR-associated9 (Cas) (CRISPR/Cas9) nuclease system, a CRISPR/Cpf1 nuclease system, a zinc finger nuclease (ZFN), a transcription activator-like effector nuclease (TALEN), a meganuclease, ribozyme, and a programmable DNA binding domain linked to a nuclease domain.

Yet another aspect of the instant disclosure encompasses method of improving the yield of an Eragrostis plant, part thereof, or plant cell thereof. The method comprises using a method of producing a lodging resistant Eragrostis plant, part thereof, or plant cell thereof to generate a genetically modified lodging resistant Eragrostis plant. The method and the genetically modified lodging resistant Eragrostis plant can be as described herein above.

Another aspect of the instant disclosure encompasses a kit for improving the yield of an Eragrostis plant, part thereof, or plant cell thereof. The kit can comprise one or more genetically modified lodging resistant Eragrostis plant, part thereof, or plant cell thereof. The genetically modified lodging resistant Eragrostis plant can be as described herein above. The kit can also comprise one or more expression constructs for introducing a nucleic acid modification in a nucleic acid sequence in an Eragrostis plant. The expression constructs can be as described herein above. The kit can also comprise one or more Eragrostis plant, part thereof, or plant cell thereof comprising one or more expression constructs for introducing a nucleic acid modification in a nucleic acid sequence in an Eragrostis plant. The expression constructs can be as described herein above.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 is a block diagram showing the main steps in the development of reduced height lodging resistant teff. The development process included two transformation steps, one to introduce targeted mutations within the Sd1 gene and a second to simultaneously introduce targeted mutations within the Dw1 and Dw3 genes. In all cases, null-segregants containing only the targeted mutations and without any introduced foreign DNA were confirmed by quantitative polymerase chain reaction (qPCR) analysis and the target site mutations confirmed by next generation sequencing (NGS).

FIG. 2 is a schematic diagram of the Sd1 (panel A), Dw1 (panel B), and Dw3 (panel C) genes on chromosomes 3, 4 and 8 of the A and B genomes. Relative locations of the gRNA target sites are shown.

FIG. 3 is a diagram showing the taxonomical classification of small millets and other major cereals and millets, and pseudo-cereals. “Millet” is a common term to categorize small-seeded grasses that are often called dryland cereals. The grasses most commonly referred to as millets are: Major millet (pearl millet) and small millets (finger millet, foxtail millet, proso millet, little millet, barnyard millet, kodo millet, browntop millet, fonio, teff and job's tears, and guinea millet).

FIG. 4 depicts an alignment of the wild-type SD1 amino acid sequences encoded by the Sd1 homoeologs on the A and B genomes (Sd1A and Sd1B) with the amino acid sequences encoded by their respective mutant alleles (sd1A1 and sd1B1) characterized in DPS 22-8-12 teff. The sd1A2 and sd1B2 encoded sequences are not shown as they are respectively the same as sd1A1 and sd1B1. The location of the codon frameshift for sd1A1 is indicated by the down-arrow (↓) and for sd1B1 by the up-arrow (↑). Sequences were aligned with MUltiple Sequence Comparison by Log-Expectation (MUSCLE) using the default parameters and displayed using TEXshade.

FIG. 5 is an of the wild-type DW1 amino acid sequences encoded by the Dw1 homoeologs on the A and B genomes (Dw1A and Dw1B) with the amino acid sequences encoded by their respective mutant alleles (dw1A1 and dw1B1) characterized in DPS 22-8-12 teff. The dw1A2 and dw1B2 encoded sequences are not shown as they are the same as dw1A1 and dw1B1, respectively. The location of the codon frameshift for dw1A1 is indicated by the down-arrow (↓) and for dw1B1 by the up-arrow (↑). Sequences were aligned with MUSCLE using the default parameters and displayed using TEXshade.

FIG. 6 is an alignment of the wild-type DW3 amino acid sequences encoded by the Dw3 homoeologs on the A and B genomes (Dw3A and Dw3B) with the amino acid sequences encoded by their respective mutant alleles (dw3A1 and dw3B1) characterized in DPS 22-8-12 teff. The dw3A2 and dw3B2 encoded sequences are not shown as they are the same as dw3A1 and dw3B1, respectively. The locations of the codon frameshift for dw1A1 is indicated by the down-arrow (↓) and for dw1B1 by the up-arrow (↑). Sequences were aligned with MUSCLE using the default parameters and displayed using TEXshade.

FIG. 7 depicts the three dimensional structure of the native teff DW3A protein (panel A) and the dw3A encoded protein containing the 611-amino acid C-terminal truncation (panel B) modeled using the Phyre2 web portal. The location of Pro-1089, which is part of the conserved D/E-P motif essential for auxin transporter activity is indicated. The entire C-terminal domain containing six additional transmembrane helices and the D/E-P signature motif is absent from the homeologous dw3-encoded sequences.

FIG. 8 is a photograph of wild-type (Sd1+Dw1+Dw3) unmodified teff with edited lines containing individual sd1, dw1, and dw3 knockout mutations, and lines with combined dw1+dw3 and sd1+dw1+dw3 mutations.

FIG. 9 is a plot showing the height of a wild type teff plant (Magna) and a genetically modified teff plant derived from the wild type plant comprising mutations in the nucleic acid sequences encoding the SD1A protein, the SD1B protein, the DW1A protein, the DW1B protein, the DW1A protein, and the DW1B protein (22-8-12).

### DETAILED DESCRIPTION

The present disclosure encompasses genetically modified lodging resistant Eragrostis plants and expression constructs and vectors for introducing the genetic modifications. The plants have a semidwarf stature and are resistant to lodging as lodging resistance negatively correlates with plant height. The genetically modified plants can be used to improve yield of teff by significantly reducing height of the plants, thereby reducing lodging and facilitating machine harvesting of Eragrostis plants such as teff.

Over 50 years of conventional breeding approaches failed to deliver lodging resistant teff varieties and to date no improved lodging resistant variety of teff is available to farmers. Significantly, the inventors developed reduced stature (dwarf or semi-dwarf) teff lines by combining multiple dwarfing genes in a single teff line using CRISPR/Cas9-mediated genome editing technology. These mutations have not been identified in existing teff germplasm and to date, this combination of mutations (SD1, DW1 and DW3) has not been attempted or reported in other cereals. Importantly, the inventors surprisingly discovered that Eragrostis plants comprising two or more of the mutations comprise a synergistic reduction in plant height when compared to a reduction in height of Eragrostis plants comprising a reduction of one of the hormone activities.

### 1. Genetically Modified Plants

One aspect of the present disclosure encompasses a genetically modified Eragrostis plant, part thereof, or plant cell thereof. The genetically modified plants comprise two or more nucleic acid modifications that result in a reduction in two or more hormone activities selected from auxin transport activity, gibberellic acid (GA) biosynthesis activity, and brassinosteroid (BR) signaling activity when compared to the hormone activities in a wild type plant. The genetically modified plants exhibit a semidwarf phenotype and are lodging resistant. Surprisingly, the disclosed semidwarf and lodging resistant phenotype was produced by a synergistic reduction in height of the Eragrostis plant.

### (a) Lodging Resistant Eragrostis Plants

As explained herein above, lodging in teff can suppress yield on average by 17 percent and substantially reduces the quality of harvested grain and straw. Currently, all improved varieties of teff lodge to a varying degree. However, decades of conventional breeding approaches to improve lodging resistance in teff has been of limited success.

Elongation of plant parts is a complex phenomenon mediated by many plant hormones. Plants contain three major growth-promoting plant hormones: auxin, gibberellins (GAs), and brassinosteroids (BRs), along with other hormones that promote growth in certain circumstances. However, the understanding of how these hormones regulate cell elongation and the pleiotropic effect of these hormones on plant height remains limited. Further, plant height can be significantly affected by genetic changes not related to hormone activities. For example, a defect in the a-tubulin gene has been shown to generate a lodging resistant dwarf teff variety of teff.

The most widely deployed and well-studied dwarfing genes are the reduced height-1 (Rht1) gene in wheat, and the semidwarf1 (sd1) mutation in rice both of which were central to the Green Revolution of the 1960s and 1970s. The wild-type alleles of these genes are involved in GA signalling (RHT1, a DELLA protein) and biosynthesis (SD1, a GA-20 oxidase). It has also been reported that the semi-dwarfism of barley, sdw1/denso, widely introgressed into cultivars, probably results from a defect in an orthologue of the rice Sd1 gene.

However, mutations in GA biosynthetic genes causing GA deficiency have not been useful in sorghum because they induce culm bending, which inevitably causes abnormal plant architecture. Rather, four independently inherited non-GA dwarfing mutations (dw1-dw4) have been used extensively in commercial grain sorghum breeding mainly in the United States to significantly reduce sorghum plant height to improve lodging resistance and machine harvesting.

The genes for three of the four dwarf loci in sorghum have been identified. Dw1 encodes a previously unknown component of BR signaling that positively regulates BR signaling by interacting with brassinosteroid insensitive 2 (BIN2) kinase and inhibiting its nuclear localization. BRs play important roles in plant growth and development, regulating diverse processes such as cell elongation, cell division, photomorphogenesis, xylem differentiation, and reproduction.

Dw2 encodes an AGC kinase involved in regulation of stem elongation. The Dw4 locus has been mapped to a region on chromosome 4, but the causal gene for dw4 has yet to be identified.

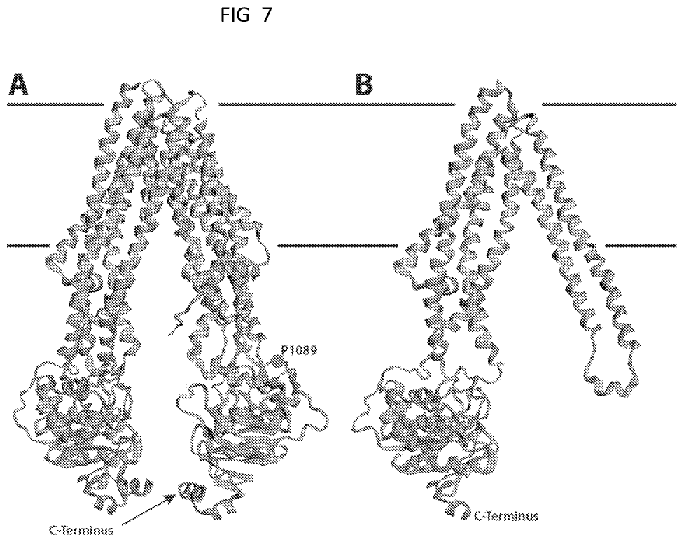

Dw3 is an orthologue to the maize brachytic2 (br2) gene, which encodes an ATP-binding cassette type B1 (ABCB1) auxin transporter. In the Arabidopsis auxin-transporting ABCB1, pro-1008 (P1008) is part of a conserved signature D/E-P motif in the C-terminal nucleotide-binding domain that seems to be specific for auxin-transporting ABCB1s. Mutation of Pro-1008 or the acidic residue (Asp or Glu) at position 1007 abolishes auxin transport activity by Arabidopsis ABCB1. All higher plant ABCB1s for which auxin transport has been conclusively proven carry the conserved D/E-P motif, including the sorghum DW3 protein. The predicted structural model for teff DW3A protein is analogous to the Arabidopsis auxin-transporting ABCB1 and contains 12 transmembrane helices and the C-terminal D/E-P motif where Pro-1089 corresponds to Pro-1008 in Arabidopsis ABCB1 (FIG. 7, panel A). The mutated teff dw3-encoded protein of the instant disclosure lacks 611 amino acids from the C-terminus that include six additional transmembrane helices and the conserved D/E-P motif essential for auxin transporter activity (FIG. 7, panel B).

A genetically modified plant, part thereof, or plant cell thereof of the instant disclosure comprises two or more nucleic acid modifications. The nucleic acid modifications result in a reduction of two or more hormone activities selected from auxin activity, gibberellic acid (GA) activity, and (BR) activity when compared to the hormone activities in a wild type plant. The genetic modification can be any nucleic acid modification in the plant that can reduce two or more of auxin activity, GA activity, and BR activity.

In some aspects, the plant comprises a nucleic acid modification that results in a reduced auxin transport activity when compared to the hormone activities in a wild type plant. In some aspects, the plant comprises a nucleic acid modification in a nucleic acid sequence encoding a DW3 protein.

In some aspects, the plant comprises a nucleic acid modification that results in a reduced GA biosynthesis activity when compared to the hormone activities in a wild type plant. In some aspects, the plant comprises a nucleic acid modification in a nucleic acid sequence encoding an SD1 protein.

In other aspects, the plant comprises a nucleic acid modification that results in a reduced BR signaling activity when compared to the hormone activities in a wild type plant. In some aspects, the plant comprises a nucleic acid modification in a nucleic acid sequence encoding a DW3 protein.

In some aspects, the genetically modified Eragrostis plant, part thereof, or plant cell thereof of the instant disclosure comprises two or more nucleic acid modifications that result in a reduction of two or more hormone activities selected from auxin transport activity, GA biosynthesis activity, and BR signaling activity when compared to the hormone activities in a wild type plant. In some aspects, an Eragrostis plant, part thereof, or plant cell thereof of the instant disclosure comprises two or more nucleic acid modifications that result in a reduction of GA biosynthesis activity and BR signaling activity when compared to the hormone activities in a wild type plant. In some aspects, an Eragrostis plant, part thereof, or plant cell thereof of the instant disclosure comprises two or more nucleic acid modifications that result in a reduction of auxin transport activity and GA biosynthesis activity when compared to the hormone activities in a wild type plant. In some aspects, an Eragrostis plant, part thereof, or plant cell thereof of the instant disclosure comprises two or more nucleic acid modifications that result in a reduction of auxin transport activity and BR signaling activity when compared to the hormone activities in a wild type plant. In some aspects, a plant, part thereof, or plant cell thereof of the instant disclosure comprises three or more nucleic acid modifications that result in a reduction of auxin transport activity, GA biosynthesis activity, and BR signaling activity when compared to the hormone activities in a wild type plant.

When the Eragrostis plant, part thereof, or plant cell thereof is teff, the nucleic acid modification that results in a reduction in a GA biosynthesis activity can be a nucleic acid modification in a nucleic acid sequence encoding an SD1A protein, a nucleic acid modification in a nucleic acid sequence encoding an SD1B protein, or both. In some aspects, the nucleic acid modification that results in a reduction in a GA biosynthesis activity is a nucleic acid modification in a nucleic acid sequence encoding an SD1A protein. In some aspects, the nucleic acid modification that results in a reduction in a GA biosynthesis activity is a nucleic acid modification in a nucleic acid sequence encoding an SD1B protein. In some aspects, the nucleic acid modification that results in a reduction in a GA biosynthesis activity is a nucleic acid modification in a nucleic acid sequence encoding an SD1B protein and a nucleic acid modification in a nucleic acid sequence encoding an SD1B protein.

In some aspects, the SD1A protein comprises an amino acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the amino acid sequence of SEQ ID NO: 17. In some aspects, the SD1A protein comprises an amino acid sequence comprising about 75% or more, at least about 85% or more, at least about 95% or more, or 100% sequence identity with the nucleic acid sequence of SEQ ID NO: 17. In some aspects, the SD1A protein is encoded by a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 18. In some aspects, the SD1A protein is encoded by a nucleic acid sequence comprising at least about 75% or more, at least about 85% or more, at least about 95% or more, or 100% sequence identity with the nucleic acid sequence of SEQ ID NO: 18.

In some aspects, the SD1B protein comprises an amino acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the amino acid sequence of SEQ ID NO: 19. In some aspects, the SD1B protein comprises an amino acid sequence comprising about 75% or more, at least about 85% or more, at least about 95% or more, or 100% sequence identity with the nucleic acid sequence of SEQ ID NO: 19. In some aspects, the SD1B protein is encoded by a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 20. In some aspects, the SD1B protein is encoded by a nucleic acid sequence comprising at least about 75% or more, at least about 85% or more, at least about 95% or more, or 100% sequence identity with the nucleic acid sequence of SEQ ID NO: 20.

In some aspects, the Eragrostis teff (teff) plant of the instant disclosure comprises a nucleic acid modification in a nucleic acid sequence encoding an SD1A protein. In some aspects, the Eragrostis teff (teff) plant of the instant disclosure comprises a nucleic acid modification in a nucleic acid sequence encoding an SD1B protein. In some aspects, the Eragrostis teff (teff) plant of the instant disclosure comprises a nucleic acid modification in a nucleic acid sequence encoding an SD1A protein and a nucleic acid modification in a nucleic acid sequence encoding an SD1B protein.

In some aspects, the nucleic acid modification in the nucleic acid sequence encoding an SD1A protein comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 21. In some aspects, the nucleic acid modification in the nucleic acid sequence encoding an SD1A protein comprises a nucleic acid sequence comprising about 75% or more, at least about 85% or more, at least about 95% or more, or 100% sequence identity with the nucleic acid sequence of SEQ ID NO: 21. In some aspects, the nucleic acid modification in the nucleic acid sequence encoding an SD1B protein comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 22. In some aspects, the nucleic acid modification in the nucleic acid sequence encoding an SD1B protein comprises a nucleic acid sequence comprising about 75% or more, at least about 85% or more, at least about 95% or more, or 100% sequence identity with the nucleic acid sequence of SEQ ID NO: 22.

When the Eragrostis plant is teff, the nucleic acid modification that results in a reduction in BR signaling activity can be a nucleic acid modification in a nucleic acid sequence encoding an DW1A protein, a nucleic acid modification in a nucleic acid sequence encoding an DW1B protein, or both. In some aspects, the nucleic acid modification that results in a reduction in BR signaling activity is a nucleic acid modification in a nucleic acid sequence encoding an DW1A protein. In some aspects, the nucleic acid modification that results in a reduction in BR signaling activity is a nucleic acid modification in a nucleic acid sequence encoding an DW1B protein. In some aspects, the nucleic acid modification that results in a reduction in BR signaling activity is a nucleic acid modification in a nucleic acid sequence encoding an DW1A protein and a nucleic acid modification in a nucleic acid sequence encoding an DW1B protein.

In some aspects, the DW1A protein comprises an amino acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the amino acid sequence of SEQ ID NO: 23. In some aspects, the SD1A protein comprises an amino acid sequence comprising about 75% or more, at least about 85% or more, at least about 95% or more, or 100% sequence identity with the nucleic acid sequence of SEQ ID NO: 23. In some aspects, the DW1A protein is encoded by a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 24. In some aspects, the DW1A protein is encoded by a nucleic acid sequence comprising at least about 75% or more, at least about 85% or more, at least about 95% or more, or 100% sequence identity with the nucleic acid sequence of SEQ ID NO: 24.

In some aspects, the DW1B protein comprises an amino acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the amino acid sequence of SEQ ID NO: 25. In some aspects, the DW1B protein comprises an amino acid sequence comprising about 75% or more, at least about 85% or more, at least about 95% or more, or 100% sequence identity with the nucleic acid sequence of SEQ ID NO: 25. In some aspects, the DW1B protein is encoded by a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 26. In some aspects, the DW1B protein is encoded by a nucleic acid sequence comprising at least about 75% or more, at least about 85% or more, at least about 95% or more, or 100% sequence identity with the nucleic acid sequence of SEQ ID NO: 26.

In some aspects, the Eragrostis teff (teff) plant of the instant disclosure comprises a nucleic acid modification in a nucleic acid sequence encoding an DW1A protein and a nucleic acid modification in a nucleic acid sequence encoding an DW1B protein. In some aspects, the nucleic acid modification in the nucleic acid sequence encoding an DW1A protein comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 27. In some aspects, the nucleic acid modification in the nucleic acid sequence encoding an DW1A protein comprises a nucleic acid sequence comprising about 75% or more, at least about 85% or more, at least about 95% or more, or 100% sequence identity with the nucleic acid sequence of SEQ ID NO: 27. In some aspects, the nucleic acid modification in the nucleic acid sequence encoding an DW1B protein comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 28. In some aspects, the nucleic acid modification in the nucleic acid sequence encoding an DW1B protein comprises a nucleic acid sequence comprising about 75% or more, at least about 85% or more, at least about 95% or more, or 100% sequence identity with the nucleic acid sequence of SEQ ID NO: 28.

When the Eragrostis plant, part thereof, or plant cell thereof is teff, the nucleic acid modification that results in a reduction in auxin transport activity can be a nucleic acid modification in a nucleic acid sequence encoding a DW3A protein, a nucleic acid modification in a nucleic acid sequence encoding a DW3B protein, or both. In some aspects, the nucleic acid modification that results in a reduction in auxin transport activity is a nucleic acid modification in a nucleic acid sequence encoding a DW3A protein. In some aspects, the nucleic acid modification that results in a reduction in auxin transport activity is a nucleic acid modification in a nucleic acid sequence encoding a DW3B protein. In some aspects, the nucleic acid modification that results in a reduction in auxin transport activity is a nucleic acid modification in a nucleic acid sequence encoding a DW3A protein and a nucleic acid modification in a nucleic acid sequence encoding a DW3B protein.

In some aspects, the DW3A protein comprises an amino acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the amino acid sequence of SEQ ID NO: 29. In some aspects, the DW3A protein comprises an amino acid sequence comprising about 75% or more, at least about 85% or more, at least about 95% or more, or 100% sequence identity with the nucleic acid sequence of SEQ ID NO: 29. In some aspects, the DW3A protein is encoded by a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 30. In some aspects, the DW3A protein is encoded by a nucleic acid sequence comprising at least about 75% or more, at least about 85% or more, at least about 95% or more, or 100% sequence identity with the nucleic acid sequence of SEQ ID NO: 30.

In some aspects, the DW3B protein comprises an amino acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the amino acid sequence of SEQ ID NO: 31. In some aspects, the DW3B protein comprises an amino acid sequence comprising about 75% or more, at least about 85% or more, at least about 95% or more, or 100% sequence identity with the nucleic acid sequence of SEQ ID NO: 31. In some aspects, the DW3B protein is encoded by a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 32. In some aspects, the DW3B protein is encoded by a nucleic acid sequence comprising at least about 75% or more, at least about 85% or more, at least about 95% or more, or 100% sequence identity with the nucleic acid sequence of SEQ ID NO: 32.

In some aspects, the Eragrostis teff (teff) plant, part thereof, or plant cell thereof of the instant disclosure comprises a nucleic acid modification in a nucleic acid sequence encoding an DW3A protein and a nucleic acid modification in a nucleic acid sequence encoding an DW3B protein. In some aspects, the nucleic acid modification in the nucleic acid sequence encoding an DW3A protein comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 33. In some aspects, the nucleic acid modification in the nucleic acid sequence encoding an DW3A protein comprises a nucleic acid sequence comprising about 75% or more, at least about 85% or more, at least about 95% or more, or 100% sequence identity with the nucleic acid sequence of SEQ ID NO: 33. In some aspects, the nucleic acid modification in the nucleic acid sequence encoding an DW3B protein comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 34. In some aspects, the nucleic acid modification in the nucleic acid sequence encoding an DW3B protein comprises a nucleic acid sequence comprising about 75% or more, at least about 85% or more, at least about 95% or more, or 100% sequence identity with the nucleic acid sequence of SEQ ID NO: 34.

In some aspects, the genetically modified lodging resistant Eragrostis tef plant comprises a nucleic acid modification in a nucleic acid sequence encoding a DW1A protein, a nucleic acid modification in a nucleic acid sequence encoding a DW1B protein, a nucleic acid modification in a nucleic acid sequence encoding a DW3A protein, and a nucleic acid modification in a nucleic acid sequence encoding a DW3B protein. In other aspects, the genetically modified lodging resistant Eragrostis tef plant comprises a nucleic acid modification in a nucleic acid sequence encoding an SD1A protein, a nucleic acid modification in a nucleic acid sequence encoding an SD1B protein, a nucleic acid modification in a nucleic acid sequence encoding a DW1A protein, a nucleic acid modification in a nucleic acid sequence encoding a DW1B protein, a nucleic acid modification in a nucleic acid sequence encoding a DW3A protein, and a nucleic acid modification in a nucleic acid sequence encoding a DW3B protein.

Surprisingly, the disclosed semidwarf and lodging resistant phenotype was produced by a synergistic reduction of plant height when two or more of mutations as described herein are combined. As used herein, the term “synergistic reduction of plant height” refers to a reduction of plant height produced when two or more of mutations as described herein are combined that is greater than the predicted reduction of plant height produced by each mutation separately.

When the Eragrostis plant is teff, the synergistic reduction in height can be about 10, 20, 30, 40, 50, 60, or about 70% when compared to wild type plants, or plants comprising one of the nucleic acid modifications. In some aspects, the synergistic reduction in height can be about 40, 45, 50, 55, 60, 65, or about 70% when compared to wild type plants, or plants comprising one of the nucleic acid modifications.

An additional aspect of the instant disclosure encompasses plants comprising one or more expression constructs as described in Section II, a vector comprising one or more expression constructs as described in Section II, or a combination thereof.

### II. Nucleotide Modification System

Another aspect of the present disclosure encompasses an expression construct for introducing a nucleic acid modification in a nucleic acid sequence in an Eragrostis plant, part thereof, or plant cell thereof. The expression construct comprises a promoter operably linked to a polynucleotide sequence encoding a programmable nucleotide modification system targeted to a nucleotide sequence in the nucleic acid sequence, wherein expression of the programmable nucleotide modification system introduces a nucleic acid modification into the nucleic acid sequence.

A programmable nucleotide modification system of the instant disclosure can introduce a nucleic acid modification in any nucleic acid sequence, wherein the nucleic acid modification results in a reduction of two or more hormone activities selected from auxin transport activity, GA biosynthesis activity, and BR signaling activity when compared to a wild type plant. In some aspects, the programmable nucleotide modification system introduces a nucleic acid modification into the nucleic acid sequence encoding an SD1 protein, a DW1 protein, or a DW3 protein.

As used herein, a “programmable nucleotide modification system” is a system capable of targeting and modifying the nucleic acid or modifying the expression or stability of a nucleic acid to alter a polynucleotide sequence or a protein or the expression of a polynucleotide sequence or protein encoded by the nucleic acid. The programmable nucleic acid modification system can comprise an interfering nucleic acid molecule or a nucleic acid editing system.

In some aspects, the programmable expression modification system comprises an interfering nucleic acid (RNAi) molecule having a nucleotide sequence complementary to a target sequence within a gene encoding the polypeptide or polynucleotide used to inhibit expression of the polypeptide or polynucleotide. RNAi molecules generally act by forming a heteroduplex with a target RNA molecule, which is selectively degraded or “knocked down,” hence inactivating the target RNA. Under some conditions, an interfering RNA molecule can also inactivate a target transcript by repressing transcript translation and/or inhibiting transcription. An interfering RNA is more generally said to be “targeted against” a biologically relevant target, such as a protein, when it is targeted against the nucleic acid encoding the target. For example, an interfering RNA molecule has a nucleotide (nt) sequence which is complementary to an endogenous mRNA of a target gene sequence. Thus, given a target gene sequence, an interfering RNA molecule can be prepared which has a nucleotide sequence at least a portion of which is complementary to a target gene sequence. When introduced into cells, the interfering RNA binds to the target mRNA, thereby functionally inactivating the target mRNA and/or leading to degradation of the target mRNA.

Interfering RNA molecules include, inter alia, small interfering RNA (siRNA), microRNA (miRNA), piwi-interacting RNA (piRNA), long non-coding RNAs (long ncRNAs or lncRNAs), and small hairpin RNAs (shRNA). lncRNAs are widely expressed and have key roles in gene regulation. Depending on their localization and their specific interactions with DNA, RNA and proteins, lncRNAs can modulate chromatin function, regulate the assembly and function of membraneless nuclear bodies, alter the stability and translation of cytoplasmic mRNAs, and interfere with signaling pathways. Piwi-interacting RNA (piRNA) is the largest class of small non-coding RNA molecules expressed in animal cells. piRNAs regulate gene expression through interactions with piwi-subfamily Argonaute proteins. SiRNA are double-stranded RNA molecules, preferably about 19-25 nucleotides in length. When transfected into cells, siRNA inhibit the target mRNA transiently until they are also degraded within the cell. MiRNA and siRNA are biochemically and functionally indistinguishable. Both are about the same in nucleotide length with 5′-phosphate and 3′-hydroxyl ends, and assemble into an RNA-induced silencing complex (RISC) to silence specific gene expression. siRNA and miRNA are distinguished based on origin. siRNA is obtained from long double-stranded RNA (dsRNA), while miRNA is derived from the double-stranded region of a 60-70 nt RNA hairpin precursor. Small hairpin RNAs (shRNA) are sequences of RNA, typically about 50-80 base pairs, or about 50, 55, 60, 65, 70, 75, or about 80 base pairs in length, that include a region of internal hybridization forming a stem loop structure consisting of a base-pair region of about 19-29 base pairs of double-strand RNA (the stem) bridged by a region of single-strand RNA (the loop) and a short 3′ overhang. shRNA molecules are processed within the cell to form siRNA which in turn knock down target gene expression. shRNA can be incorporated into plasmid vectors and integrated into genomic DNA for longer-term or stable expression, and thus longer knockdown of the target mRNA.

Interfering nucleic acid molecules can contain RNA bases, non-RNA bases, or a mixture of RNA bases and non-RNA bases. For example, interfering nucleic acid molecules provided herein can be primarily composed of RNA bases but also contain DNA bases or non-naturally occurring nucleotides. The interfering nucleic acids can employ a variety of oligonucleotide chemistries. Examples of oligonucleotide chemistries include, without limitation, peptide nucleic acid (PNA), linked nucleic acid (LNA), phosphorothioate, 2′O-Me-modified oligonucleotides, and morpholino chemistries, including combinations of any of the foregoing. In general, PNA and LNA chemistries can utilize shorter targeting sequences because of their relatively high target binding strength relative to 2′O-Me oligonucleotides. Phosphorothioate and 2′O-Me-modified chemistries are often combined to generate 2′O-Me-modified oligonucleotides having a phosphorothioate backbone.

In some aspects, the programmable nucleotide modification system is a nucleic acid editing system. Such modification system can be used to edit DNA or RNA sequences to repress transcription or translation of an mRNA encoded by the gene, and/or produce mutant proteins with reduced activity or stability. Non-limiting examples of programmable nucleic acid editing systems include, without limit, an RNA-guided clustered regularly interspersed short palindromic repeats (CRISPR)/CRISPR-associated (Cas) (CRISPR/Cas) nuclease system, a CRISPR/Cpf1 nuclease system, a zinc finger nuclease (ZFN), a transcription activator-like effector nuclease (TALEN), a meganuclease, a ribozyme, or a programmable DNA binding domain linked to a nuclease domain. Other suitable programmable nucleic acid modification systems will be recognized by individuals skilled in the art.

Such systems rely for specificity on the delivery of exogenous protein(s), and/or a guide RNA (gRNA) or single guide RNA (sgRNA) having a sequence which binds specifically to a gene sequence of interest. When the programmable nucleic acid modification system comprises more than one component, such as a protein and a guide nucleic acid, the multi-component modification system can be modular, in that the different components may optionally be distributed among two or more nucleic acid constructs as described herein. The system components can be delivered by a plasmid or viral vector or as a synthetic oligonucleotide. More detailed descriptions of programmable nucleic acid editing systems can be as described further below.

In some aspects, the programmable nucleic acid modification system is a CRISPR/Cas tool modified for transcriptional regulation of a locus. In some aspects, the programmable nucleic acid modification system is CRISPR/Cas system comprising a Cas9 nuclease and a guide RNA (gRNA) comprising a sequence complementary to a target sequence within the nucleotide sequence encoding an SD1 protein, a DW1 protein, or a DW3 protein.

In some aspects, the genetically modified Eragrostis plant, part thereof, or plant cell thereof is teff. When the plant is teff, the programmable nucleotide modification system of the instant disclosure can introduce a nucleic acid modification in a nucleic acid sequence encoding an SD1A protein, a nucleic acid sequence encoding an SD1B protein, or both. In some aspects, the programmable nucleotide modification system introduces a nucleic acid modification in a nucleic acid sequence encoding an SD1A protein. When the programmable nucleotide modification system is a CRISPR/Cas system and the polypeptide is an SD1A protein, the gRNA can comprise a nucleic acid sequence of SEQ ID NO: 35 (Sd1 gRNA1), SEQ ID NO: 36 (Sd1 gRNA2), or a combination thereof.

In other aspects, the programmable nucleotide modification system introduces a nucleic acid modification in a nucleic acid sequence encoding an SD1B protein. When the programmable nucleotide modification system is a CRISPR/Cas system and the polypeptide is an SD1B protein, the gRNA can comprise a nucleic acid sequence of SEQ ID NO: 35 (Sd1 gRNA1), SEQ ID NO: 36 (Sd1 gRNA2), or a combination thereof.

In yet other aspects, the programmable nucleotide modification system introduces a nucleic acid modification in a nucleic acid sequence encoding an SD1A protein and in a nucleic acid sequence encoding an SD1B protein. When the programmable nucleotide modification system is a CRISPR/Cas system and the polypeptide is an SD1A protein and an SD1B protein, the gRNA can comprise a nucleic acid sequence of SEQ ID NO: 35 (Sd1 gRNA1), SEQ ID NO: 36 (Sd1 gRNA2), or a combination thereof.

When the plant is teff, the programmable nucleotide modification system of the instant disclosure can introduce a nucleic acid modification in a nucleic acid sequence encoding a DW1A protein, a nucleic acid sequence encoding a DW1B protein, or both. In some aspects, the programmable nucleotide modification system introduces a nucleic acid modification in a nucleic acid sequence encoding an DW1A protein. When the programmable nucleotide modification system is a CRISPR/Cas system and the polypeptide is an DW1A protein, the gRNA can comprise a nucleic acid sequence of SEQ ID NO: 37 (Dw1 gRNA).

In other aspects, the programmable nucleotide modification system introduces a nucleic acid modification in a nucleic acid sequence encoding an DW1B protein. When the programmable nucleotide modification system is a CRISPR/Cas system and the polypeptide is an DW1B protein, the gRNA can comprise a nucleic acid sequence of SEQ ID NO: 37 (Dw3 gRNA).

In yet other aspects, the programmable nucleotide modification system introduces a nucleic acid modification in a nucleic acid sequence encoding an DW1A protein and in a nucleic acid sequence encoding an DW1B protein. When the programmable nucleotide modification system is a CRISPR/Cas system and the polypeptide is an DW1A protein and an DW1B protein, the gRNA can comprise a nucleic acid sequence of SEQ ID NO: 37 (Dw1 gRNA).

When the plant is teff, the programmable nucleotide modification system of the instant disclosure can introduce a nucleic acid modification in a nucleic acid sequence encoding a DW3A protein, a nucleic acid sequence encoding an DW3B protein, or both. In some aspects, the programmable nucleotide modification system introduces a nucleic acid modification in a nucleic acid sequence encoding an DW3A protein. When the programmable nucleotide modification system is a CRISPR/Cas system and the polypeptide is a DW3A protein, the gRNA can comprise a nucleic acid sequence of SEQ ID NO: 38 (Dw3 gRNA).

In other aspects, the programmable nucleotide modification system introduces a nucleic acid modification in a nucleic acid sequence encoding an DW3B protein. When the programmable nucleotide modification system is a CRISPR/Cas system and the polypeptide is an DW3B protein, the gRNA can comprise a nucleic acid sequence of SEQ ID NO: 38 (Dw3 gRNA).

In yet other aspects, the programmable nucleotide modification system introduces a nucleic acid modification in a nucleic acid sequence encoding an DW3A protein and in a nucleic acid sequence encoding an DW3B protein. When the programmable nucleotide modification system is a CRISPR/Cas system and the polypeptide is an DW3A protein and an DW3B protein, the gRNA can comprise a nucleic acid sequence of SEQ ID NO: 38 (Dw3 gRNA).

Other nucleic acid sequences of components of the nucleotide modification system are described in Section III herein below.

i. CRISPR Nuclease Systems.

The programmable targeting nuclease can be an RNA-guided CRISPR endonuclease system. The CRISPR system comprises a guide RNA or sgRNA to a target sequence at which a protein of the system introduces a double-stranded break in a target nucleic acid sequence, and a CRISPR-associated endonuclease. The gRNA is a short synthetic RNA comprising a sequence necessary for endonuclease binding, and a preselected ˜20 nucleotide spacer sequence targeting the sequence of interest in a genomic target. Non-limiting examples of endonucleases include Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 endonuclease, or a homolog thereof, a recombination of the naturally occurring molecule thereof, a codon-optimized version thereof, or a modified version thereof, or any combination thereof.

The CRISPR nuclease system may be derived from any type of CRISPR system, including a type I (i.e., IA, IB, IC, ID, IE, or IF), type II (i.e., IIA, IIB, or IIC), type III (i.e., IIIA or IIIB), or type V CRISPR system. The CRISPR/Cas system may be from Streptococcus sp. (e.g., Streptococcus pyogenes), Campylobacter sp. (e.g., Campylobacter jejuni), Francisella sp. (e.g., Francisella novicida), Acaryochloris sp., Acetohalobium sp., Acidaminococcus sp., Acidithiobacillus sp., Alicyclobacillus sp., Allochromatium sp., Ammonifex sp., Anabaena sp., Arthrospira sp., Bacillus sp., Burkholderiales sp., Caldicelulosiruptor sp., Candidatus sp., Clostridium sp., Crocosphaera sp., Cyanothece sp., Exiguobacterium sp., Finegoldia sp., Ktedonobacter sp., Lactobacillus sp., Lyngbya sp., Marinobacter sp., Methanohalobium sp., Microscilla sp., Microcoleus sp., Microcystis sp., Natranaerobius sp., Neisseria sp., Nitrosococcus sp., Nocardiopsis sp., Nodularia sp., Nostoc sp., Oscillatoria sp., Polaromonas sp., Pelotomaculum sp., Pseudoalteromonas sp., Petrotoga sp., Prevotella sp., Staphylococcus sp., Streptomyces sp., Streptosporangium sp., Synechococcus sp., or Thermosipho sp.

Non-limiting examples of suitable CRISPR systems include CRISPR/Cas systems, CRISPR/Cpf systems, CRISPR/Cmr systems, CRISPR/Csa systems, CRISPR/Csb systems, CRISPR/Csc systems, CRISPR/Cse systems, CRISPR/Csf systems, CRISPR/Csm systems, CRISPR/Csn systems, CRISPR/Csx systems, CRISPR/Csy systems, CRISPR/Csz systems, and derivatives or variants thereof. Preferably, the CRISPR system may be a type II Cas9 protein, a type V Cpf1 protein, or a derivative thereof. In some aspects, the CRISPR/Cas nuclease is Streptococcus pyogenes Cas9 (SpCas9), Streptococcus thermophilus Cas9 (StCas9), Campylobacter jejuni Cas9 (CjCas9), Francisella novicida Cas9 (FnCas9), or Francisella novicida Cpf1 (FnCpf1).

In general, a protein of the CRISPR system comprises an RNA recognition and/or RNA binding domain, which interacts with the guide RNA. A protein of the CRISPR system also comprises at least one nuclease domain having endonuclease activity. For example, a Cas9 protein may comprise a RuvC-like nuclease domain and an HNH-like nuclease domain, and a Cpf1 protein may comprise a RuvC-like domain. A protein of the CRISPR system may also comprise DNA binding domains, helicase domains, RNase domains, protein-protein interaction domains, dimerization domains, as well as other domains.

A protein of the CRISPR system may be associated with guide RNAs (gRNA). The guide RNA may be a single guide RNA (i.e., sgRNA), or may comprise two RNA molecules (i.e., crRNA and tracrRNA). The guide RNA interacts with a protein of the CRISPR system to guide it to a target site in the DNA. The target site has no sequence limitation except that the sequence is bordered by a protospacer adjacent motif (PAM). For example, PAM sequences for Cas9 include 3′-NGG, 3′-NGGNG, 3′-NNAGAAW, and 3′-ACAY, and PAM sequences for Cpf1 include 5′-TTN (wherein N is defined as any nucleotide, W is defined as either A or T, and Y is defined as either C or T). Each gRNA comprises a sequence that is complementary to the target sequence (e.g., a Cas9 gRNA may comprise GN17-20GG). The gRNA may also comprise a scaffold sequence that forms a stem loop structure and a single-stranded region. The scaffold region may be the same in every gRNA. In some aspects, the gRNA may be a single molecule (i.e., sgRNA). In other aspects, the gRNA may be two separate molecules. Those skilled in the art are familiar with gRNA design and construction, e.g., gRNA design tools are available on the internet or from commercial sources.

A CRISPR system may comprise one or more nucleic acid binding domains associated with one or more, or two or more selected guide RNAs used to direct the CRISPR system to one or more, or two or more selected target nucleic acid loci. For instance, a nucleic acid binding domain may be associated with one or more, or two or more selected guide RNAs, each selected guide RNA, when complexed with a nucleic acid binding domain, causing the CRISPR system to localize to the target of the guide RNA.

ii. CRISPR Nickase Systems.

The programmable targeting nuclease can also be a CRISPR nickase system. CRISPR nickase systems are similar to the CRISPR nuclease systems described above except that a CRISPR nuclease of the system is modified to cleave only one strand of a double-stranded nucleic acid sequence. Thus, a CRISPR nickase, in combination with a guide RNA of the system, may create a single-stranded break or nick in the target nucleic acid sequence. Alternatively, a CRISPR nickase in combination with a pair of offset gRNAs may create a double-stranded break in the nucleic acid sequence.

A CRISPR nuclease of the system may be converted to a nickase by one or more mutations and/or deletions. For example, a Cas9 nickase may comprise one or more mutations in one of the nuclease domains, wherein the one or more mutations may be D10A, E762A, and/or D986A in the RuvC-like domain, or the one or more mutations may be H840A (or H839A), N854A and/or N863A in the HNH-like domain.

iii. ssDNA-Guided Argonaute Systems.

Alternatively, the programmable targeting nuclease may comprise a single-stranded DNA-guided Argonaute endonuclease. Argonautes (Agos) are a family of endonucleases that use 5′-phosphorylated short single-stranded nucleic acids as guides to cleave nucleic acid targets. Some prokaryotic Agos use single-stranded guide DNAs and create double-stranded breaks in nucleic acid sequences. The ssDNA-guided Ago endonuclease may be associated with a single-stranded guide DNA.

The Ago endonuclease may be derived from Alistipes sp., Aquifex sp., Archaeoglobus sp., Bacteroides sp., Bradyrhizobium sp., Burkholderia sp., Cellvibrio sp., Chlorobium sp., Geobacter sp., Mariprofundus sp., Natronobacterium sp., Parabacteriodes sp., Parvularcula sp., Planctomyces sp., Pseudomonas sp., Pyrococcus sp., Thermus sp., or Xanthomonas sp. For instance, the Ago endonuclease may be Natronobacterium gregoryi Ago (NgAgo). Alternatively, the Ago endonuclease may be Thermus thermophilus Ago (TtAgo). The Ago endonuclease may also be Pyrococcus furiosus (PfAgo).

The single-stranded guide DNA (gDNA) of an ssDNA-guided Argonaute system is complementary to the target site in the nucleic acid sequence. The target site has no sequence limitations and does not require a PAM. The gDNA generally ranges in length from about 15-30 nucleotides. The gDNA may comprise a 5′ phosphate group. Those skilled in the art are familiar with ssDNA oligonucleotide design and construction.

iv. Zinc Finger Nucleases.

The programmable targeting nuclease may be a zinc finger nuclease (ZFN). A ZFN comprises a DNA-binding zinc finger region and a nuclease domain. The zinc finger region may comprise from about two to seven zinc fingers, for example, about four to six zinc fingers, wherein each zinc finger binds three nucleotides. The zinc finger region may be engineered to recognize and bind to any DNA sequence. Zinc finger design tools or algorithms are available on the internet or from commercial sources. The zinc fingers may be linked together using suitable linker sequences.

A ZFN also comprises a nuclease domain, which may be obtained from any endonuclease or exonuclease. Non-limiting examples of endonucleases from which a nuclease domain may be derived include, but are not limited to, restriction endonucleases and homing endonucleases. The nuclease domain may be derived from a type II-S restriction endonuclease. Type II-S endonucleases cleave DNA at sites that are typically several base pairs away from the recognition/binding site and, as such, have separable binding and cleavage domains. These enzymes generally are monomers that transiently associate to form dimers to cleave each strand of DNA at staggered locations. Non-limiting examples of suitable type II-S endonucleases include BfiI, BpmI, BsaI, BsgI, BsmBI, BsmI, BspMI, FokI, MbolI, and SapI. The type II-S nuclease domain may be modified to facilitate dimerization of two different nuclease domains. For example, the cleavage domain of FokI may be modified by mutating certain amino acid residues. By way of non-limiting example, amino acid residues at positions 446, 447, 479, 483, 484, 486, 487, 490, 491, 496, 498, 499, 500, 531, 534, 537, and 538 of FokI nuclease domains are targets for modification. For example, one modified FokI domain may comprise Q486E, I499L, and/or N496D mutations, and the other modified FokI domain may comprise E490K, 1538K, and/or H537R mutations.

v. Transcription Activator-Like Effector Nuclease Systems.

The programmable targeting nuclease may also be a transcription activator-like effector nuclease (TALEN) or the like. TALENs comprise a DNA-binding domain composed of highly conserved repeats derived from transcription activator-like effectors (TALEs) that are linked to a nuclease domain. TALEs are proteins secreted by plant pathogen Xanthomonas to alter transcription of genes in host plant cells. TALE repeat arrays may be engineered via modular protein design to target any DNA sequence of interest. Other transcription activator-like effector nuclease systems may comprise, but are not limited to, the repetitive sequence, transcription activator like effector (RipTAL) system from the bacterial plant pathogenic Ralstonia solanacearum species complex (Rssc). The nuclease domain of TALEs may be any nuclease domain as described above in Section II(i).

vi. Meganucleases or Rare-Cutting Endonuclease Systems.

The programmable targeting nuclease may also be a meganuclease or derivative thereof. Meganucleases are endodeoxyribonucleases characterized by long recognition sequences, i.e., the recognition sequence generally ranges from about 12 base pairs to about 45 base pairs. As a consequence of this requirement, the recognition sequence generally occurs only once in any given genome. Among meganucleases, the family of homing endonucleases named LAGLIDADG has become a valuable tool for the study of genomes and genome engineering. Non-limiting examples of meganucleases that may be suitable for the instant disclosure include I-SceI, I-CreI, I-DmoI, or variants and combinations thereof. A meganuclease may be targeted to a specific nucleic acid sequence by modifying its recognition sequence using techniques well known to those skilled in the art.

The programmable targeting nuclease can be a rare-cutting endonuclease or derivative thereof. Rare-cutting endonucleases are site-specific endonucleases whose recognition sequence occurs rarely in a genome, such as only once in a genome. The rare-cutting endonuclease may recognize a 7-nucleotide sequence, an 8-nucleotide sequence, or longer recognition sequence. Non-limiting examples of rare-cutting endonucleases include NotI, AscI, PacI, AsiSI, SbfI, and FseI.

vii. Optional Additional Domains.

The programmable targeting nuclease may further comprise at least one nuclear localization signal (NLS), at least one cell-penetrating domain, at least one reporter domain, and/or at least one linker.

In general, an NLS comprises a stretch of basic amino acids. Nuclear localization signals are known in the art (see, e.g., Lange et al., J. Biol. Chem., 2007, 282:5101-5105). The NLS may be located at the N-terminus, the C-terminal, or in an internal location of the fusion protein.

A cell-penetrating domain may be a cell-penetrating peptide sequence derived from the HIV-1 TAT protein. The cell-penetrating domain may be located at the N-terminus, the C-terminal, or in an internal location of the fusion protein.

A programmable targeting nuclease may further comprise at least one linker. For example, the programmable targeting nuclease, the nuclease domain of the targeting nuclease, and other optional domains may be linked via one or more linkers. The linker may be flexible (e.g., comprising small, non-polar (e.g., Gly) or polar (e.g., Ser, Thr) amino acids). Examples of suitable linkers are well known in the art, and programs to design linkers are readily available (Crasto et al., Protein Eng., 2000, 13(5):3096-312). In alternate aspects, the programmable targeting nuclease, the cell cycle regulated protein, and other optional domains may be linked directly.

A programmable targeting nuclease may further comprise an organelle localization or targeting signal that directs a molecule to a specific organelle. A signal may be a polynucleotide or polypeptide signal, or may be an organic or inorganic compound sufficient to direct an attached molecule to a desired organelle. Organelle localization signals can be as described in U.S. Patent Publication No. 20070196334, the disclosure of which is incorporated herein in its entirety.

### III. Nucleic Acid Constructs

A further aspect of the present disclosure provides a system of one or more nucleic acid constructs encoding the components of the programmable nucleotide modification system described above in Section II.

Any of the multi-component systems described herein are to be considered modular, in that the different components may optionally be distributed among two or more nucleic acid constructs as described herein. The nucleic acid constructs may be DNA or RNA, linear or circular, single-stranded or double-stranded, or any combination thereof. The nucleic acid constructs may be codon-optimized for efficient translation into protein, and possibly for transcription into an RNA donor polynucleotide transcript in the cell of interest. Codon optimization programs are available as freeware or from commercial sources.

The nucleic acid constructs can be used to express one or more components of the system for later introduction into a cell to be genetically modified. Alternatively, the nucleic acid constructs can be introduced into the cell to be genetically modified for expression of the components of the system in the cell. In some aspects, the nucleic acid constructs transiently express the various components of the system. Transiently expressing the system in a plant overcomes the cumbersome regulatory hurdles required for traditionally genetically modified crops.

Expression constructs generally comprise DNA coding sequences operably linked to at least one promoter control sequence for expression in a cell of interest. Promoter control sequences may control expression of the transposase, the programmable targeting nuclease, the donor polynucleotide, or combinations thereof in bacterial (e.g., E. coli) cells or eukaryotic (e.g., yeast, insect, mammalian, or plant) cells. Suitable bacterial promoters include, without limit, T7 promoters, lac operon promoters, trp promoters, tac promoters (which are hybrids of trp and lac promoters), variations of any of the foregoing, and combinations of any of the foregoing. Non-limiting examples of suitable eukaryotic promoters include constitutive, regulated, or cell- or tissue-specific promoters.

Suitable eukaryotic constitutive promoter control sequences include, but are not limited to, cytomegalovirus immediate early promoter (CMV), simian virus (SV40) promoter, adenovirus major late promoter, Rous sarcoma virus (RSV) promoter, mouse mammary tumor virus (MMTV) promoter, phosphoglycerate kinase (PGK) promoter, elongation factor (ED1)-alpha promoter, ubiquitin promoters, actin promoters, tubulin promoters, immunoglobulin promoters, fragments thereof, or combinations of any of the foregoing. Examples of suitable eukaryotic regulated promoter control sequences include, without limit, those regulated by heat shock, metals, steroids, antibiotics, or alcohol. Non-limiting examples of tissue-specific promoters include B29 promoter, CD14 promoter, CD43 promoter, CD45 promoter, CD68 promoter, desmin promoter, elastase-1 promoter, endoglin promoter, fibronectin promoter, Flt-1 promoter, GFAP promoter, GPIIb promoter, ICAM-2 promoter, INF-3 promoter, Mb promoter, NphsI promoter, OG-2 promoter, SP-B promoter, SYN1 promoter, and WASP promoter.

Promoters can also be plant-specific promoters, or promoters that may be used in plants. A wide variety of plant promoters are known to those of ordinary skill in the art, as are other regulatory elements that may be used alone or in combination with promoters. Preferably, promoter control sequences control expression in cassava, such as promoters disclosed in Wilson et al., 2017, The New Phytologist, 213(4):1632-1641, the disclosure of which is incorporated herein in its entirety.

Promoters can be divided into two types, namely, constitutive promoters and non-constitutive promoters. Constitutive promoters are classified as providing for a range of constitutive expression. Thus, some are weak constitutive promoters, and others are strong constitutive promoters. Non-constitutive promoters include tissue-preferred promoters, tissue-specific promoters, cell-type specific promoters, and inducible promoters. Suitable plant-specific constitutive promoter control sequences include, but are not limited to, a CaMV35S promoter, CaMV 19S, GOS2, Arabidopsis At6669 promoter, Rice cyclophilin, Maize H3 histone, Synthetic Super MAS, an opine promoter, a plant ubiquitin (Ubi) promoter, an actin 1 (Act-1) promoter, pEMU, Cestrum yellow leaf curling virus promoter (CYMLV promoter), and an alcohol dehydrogenase 1 (Adh-1) promoter. Other constitutive promoters include those in U.S. Pat. Nos. 5,659,026; 5,608,149; 5,608,144; 5,604,121; 5,569,597; 5,466,785; 5,399,680; 5,268,463; and 5,608,142.

Regulated plant promoters respond to various forms of environmental stresses, or other stimuli, including, for example, mechanical shock, heat, cold, flooding, drought, salt, anoxia, pathogens such as bacteria, fungi, and viruses, and nutritional deprivation, including deprivation during times of flowering and/or fruiting, and other forms of plant stress. For example, the promoter may be a promoter which is induced by one or more, but not limited to one of the following: abiotic stresses such as wounding, cold, desiccation, ultraviolet-B, heat shock or other heat stress, drought stress or water stress. The promoter may further be one induced by biotic stresses including pathogen stress, such as stress induced by a virus or fungi, stresses induced as part of the plant defense pathway or by other environmental signals, such as light, carbon dioxide, hormones or other signaling molecules such as auxin, hydrogen peroxide and salicylic acid, sugars and gibberellin or abscisic acid and ethylene. Suitable regulated plant promoter control sequences include, but are not limited to, salt-inducible promoters such as RD29A; drought-inducible promoters such as maize rab17 gene promoter, maize rab28 gene promoter, and maize Ivr2 gene promoter; heat-inducible promoters such as heat tomato hsp80-promoter from tomato.

Tissue-specific promoters may include, but are not limited to, fiber-specific, green tissue-specific, root-specific, stem-specific, flower-specific, callus-specific, pollen-specific, egg-specific, and seed coat-specific. Suitable tissue-specific plant promoter control sequences include, but are not limited to, leaf-specific promoters [such as described, for example, by Yamamoto et al., Plant J. 12:255-265, 1997; Kwon et al., Plant Physiol. 105:357-67, 1994; Yamamoto et al., Plant Cell Physiol. 35:773-778, 1994; Gotor et al., Plant J. 3:509-18, 1993; Orozco et al., Plant Mol. Biol. 23:1129-1138, 1993; and Matsuoka et al., Proc. Natl. Acad. Sci. USA 90:9586-9590, 1993], seed-preferred promoters [e.g., from seed-specific genes (Simon et al., Plant Mol. Biol. 5. 191, 1985; Scofield et al., J. Biol. Chem. 262: 12202, 1987; Baszczynski et al., Plant Mol. Biol. 14: 633, 1990), Brazil Nut albumin (Pearson et al., Plant Mol. Biol. 18: 235-245, 1992), legumin (Ellis et al., Plant Mol. Biol. 10: 203-214, 1988), Glutelin (rice) (Takaiwa et al., Mol. Gen. Genet. 208: 15-22, 1986; Takaiwa et al., FEBS Letts. 221: 43-47, 1987), Zein (Matzke et al., Plant Mol Biol, 143: 323-32, 1990), napA (Stalberg et al., Planta 199: 515-519, 1996), Wheat SPA (Albani et al, Plant Cell, 9: 171-184, 1997), sunflower oleosin (Cummins et al., Plant Mol. Biol. 19: 873-876, 1992)], endosperm specific promoters [e.g., wheat LMW and HMW, glutenin-1 (Mol Gen Genet 216:81-90, 1989; NAR 17:461-2), wheat a, b, and g gliadins (EMB03:1409-15, 1984), Barley Itrl promoter, barley B1, C, D hordein (Theor Appl Gen 98:1253-62, 1999; Plant J 4:343-55, 1993; Mol Gen Genet 250:750-60, 1996), Barley DOF (Mena et al., The Plant Journal, 116(1): 53-62, 1998), Biz2 (EP99106056.7), Synthetic promoter (Vicente-Carbajosa et al., Plant J. 13: 629-640, 1998), rice prolamin NRP33, rice-globulin Glb-1 (Wu et al., Plant Cell Physiology 39(8) 885-889, 1998), rice alpha-globulin REB/OHP-1 (Nakase et al., Plant Mol. Biol. 33: 513-S22, 1997), rice ADP-glucose PP (Trans Res 6:157-68, 1997), maize ESR gene family (Plant J 12:235-46, 1997), sorgum gamma-kafirin (PMB 32:1029-35, 1996)], embryo-specific promoters [e.g., rice OSH1 (Sato et al., Proc. Natl. Acad. Sci. USA, 93: 8117-8122), KNOX (Postma-Haarsma et al., Plant Mol. Biol. 39:257-71, 1999), rice oleosin (Wu et al., J. Biochem., 123:386, 1998)], and flower-specific promoters [e.g., AtPRP4, chalene synthase (chsA) (Van der Meer et al., Plant Mol. Biol. 15, 95-109, 1990), LAT52 (Twell et al., Mol. Gen Genet. 217:240-245; 1989), apetala-3].

Any of the promoter sequences can be wild type or may be modified for more efficient or efficacious expression. The DNA coding sequence also can be linked to a polyadenylation signal (e.g., SV40 polyA signal, bovine growth hormone (BGH) polyA signal, etc.) and/or at least one transcriptional termination sequence. In some situations, the complex or fusion protein can be purified from the bacterial or eukaryotic cells.

Nucleic acids encoding one or more components of programmable nucleotide modification system can be present in a construct. Suitable constructs include plasmid constructs, viral constructs, and self-replicating RNA (Yoshioka et al., Cell Stem Cell, 2013, 13:246-254). For instance, the nucleic acid encoding one or more components of a programmable nucleotide modification system may be present in a plasmid construct.

Non-limiting examples of suitable plasmid constructs include pUC, pBR322, pET, pBluescript, and variants thereof. Alternatively, the nucleic acid encoding one or more components of a programmable nucleotide modification system can be part of a viral vector (e.g., lentiviral vectors, adeno-associated viral vectors, adenoviral vectors, and so forth).

The plasmid or viral vector can comprise additional expression control sequences (e.g., enhancer sequences, Kozak sequences, polyadenylation sequences, transcriptional termination sequences, etc.), selectable reporter sequences (e.g., antibiotic resistance genes), origins of replication, T-DNA border sequences, and the like. The plasmid or viral vector may further comprise RNA processing elements such as glycine tRNAs, or Csy4 recognition sites. Such RNA processing elements can, for instance, intersperse polynucleotide sequences encoding multiple gRNAs under the control of a single promoter to produce the multiple gRNAs from a transcript encoding the multiple gRNAs. When a cys4 recognition cite is used, a vector may further comprise sequences for expression of Csy4 RNAse to process the gRNA transcript. Additional information about vectors and use thereof may be found in “Current Protocols in Molecular Biology”, Ausubel et al., John Wiley & Sons, New York, 2003, or “Molecular Cloning: A Laboratory Manual”, Sambrook & Russell, Cold Spring Harbor Press, Cold Spring Harbor, NY, 3rd edition, 2001.

The plasmid or viral vector can also comprise a transit peptide for targeting of a protein product, particularly to a chloroplast, leucoplast or other plastid organelle or vacuole or an extracellular location. For descriptions of the use of chloroplast transit peptides, see U.S. Pat. Nos. 5,188,642 and 5,728,925, herein incorporated by reference in their entirety. Many chloroplast-localized proteins are expressed from nuclear genes as precursors and are targeted to the chloroplast by a chloroplast transit peptide (CTP). Examples of other such isolated chloroplast proteins include, but are not limited to those associated with the small subunit (SSU) of ribulose-1,5,-bisphosphate carboxylase, ferredoxin, ferredoxin oxidoreductase, the light-harvesting complex protein I and protein II, thioredoxin F, enolpyruvyl shikimate phosphate synthase (EPSPS) and transit peptides described in U.S. Pat. No. 7,193,133, herein incorporated by reference. It has been demonstrated in vivo and in vitro that non-chloroplast proteins may be targeted to the chloroplast by use of protein fusions with a heterologous CTP and that the CTP is sufficient to target a protein to the chloroplast. Incorporation of a suitable chloroplast transit peptide, such as, the Arabidopsis thaliana EPSPS CTP (CTP2, Klee et al., Mol. Gen. Genet. 210:437-442), and the Petunia hybrida EPSPS CTP (CTP4, della-Cioppa et al., Proc. Natl. Acad. Sci. USA 83:6873-6877) has been show to target heterologous EPSPS protein sequences to chloroplasts in transgenic plants. The production of glyphosate tolerant plants by expression of a fusion protein comprising an amino-terminal CTP with a glyphosate resistant EPSPS enzyme is well known by those skilled in the art, (U.S. Pat. Nos. 5,627,061, 5,633,435, 5,312,910, EP 0218571, EP 189707, EP 508909, and EP 924299).

In some aspects, the nucleic acid construct comprises an expression construct comprising a promoter operably linked to a polynucleotide sequence encoding a programmable nucleotide modification system targeted to a polynucleotide in the nucleic acid sequence encoding an SD1 protein, a DW1 protein, or a DW3 protein, wherein expression of the programmable nucleotide modification system introduces a nucleic acid modification into the nucleic acid sequence, and wherein the nucleic acid modification in the nucleic acid sequence encoding an SD1 protein results in a reduction in GA biosynthesis activity, the nucleic acid modification in the nucleic acid sequence encoding a DW1 protein results in a reduction in BR signaling activity, and the nucleic acid modification in the nucleic acid sequence encoding a DW3 protein results in a reduction in auxin transport activity.

In some aspects, when the Eragrostis plant, part thereof, or plant cell thereof is Eragrostis tef plant, part thereof, or plant cell thereof. In some aspects, when the Eragrostis plant, part thereof, or plant cell thereof is Eragrostis tef plant, part thereof, or plant cell thereof, a nucleic acid construct encoding the components of the programmable nucleotide modification system targeted to a polynucleotide in a nucleic acid sequence encoding an SD1 protein. In some aspects, nucleic acid construct encoding the components of the programmable nucleotide modification system targeted to a polynucleotide in a nucleic acid sequence encoding an SD1 protein comprises about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 39. In some aspects, nucleic acid construct encoding the components of the programmable nucleotide modification system targeted to a polynucleotide in a nucleic acid sequence encoding an SD1 protein comprises about 95% or more, or 100% sequence identity with the nucleic acid sequence of SEQ ID NO: 39.

In some aspects, when the Eragrostis plant, part thereof, or plant cell thereof is Eragrostis tef plant, part thereof, or plant cell thereof. In some aspects, when the Eragrostis plant, part thereof, or plant cell thereof is Eragrostis tef plant, part thereof, or plant cell thereof, a nucleic acid construct encoding the components of the programmable nucleotide modification system targeted to a polynucleotide in a nucleic acid sequence encoding a DW1 protein and a nucleic acid construct encoding the components of the programmable nucleotide modification system targeted to a polynucleotide in a nucleic acid sequence encoding a DW3 protein. In some aspects, a nucleic acid construct encoding the components of the programmable nucleotide modification system targeted to a polynucleotide in a nucleic acid sequence encoding a DW1 protein and a nucleic acid construct encoding the components of the programmable nucleotide modification system targeted to a polynucleotide in a nucleic acid sequence encoding a DW3 protein comprises about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 40. In some aspects, nucleic acid construct encoding the components of the programmable nucleotide modification system targeted to a polynucleotide in a nucleic acid sequence encoding an SD1 protein comprises about 95% or more, or 100% sequence identity with the nucleic acid sequence of SEQ ID NO: 40.

### IV. Methods

A further aspect of the present disclosure encompasses a method of producing a lodging resistant Eragrostis plant, part thereof, or plant cell thereof, the method comprising introducing into the plant two or more nucleic acid modifications that result in a reduction in two or more plant hormone activities selected from auxin transport activity, gibberellic acid (GA) biosynthesis activity, and brassinosteroid (BR) signaling activity. Genetically modified plants generated using methods of the instant disclosure can be as described in Section I herein above.

The method comprises introducing a nucleic acid modification into the plant. The genetic modification can comprise an exogenous nucleic acid molecule such as a chimeric nucleic acid of the disclosure. The term “exogenous” as used herein refers to a nucleic acid molecule originating from outside the plant cell. An exogenous nucleic acid molecule can be, for example, the coding sequence of a nucleic acid molecule encoding a factor associated with a plant hormone activity, or an element which reduces expression of a hormone activity selected from auxin transport activity, gibberellic acid (GA) biosynthesis activity, and brassinosteroid (BR) signaling activity. An exogenous nucleic acid molecule can have a naturally occurring or non-naturally occurring nucleotide sequence and can be a heterologous nucleic acid molecule derived from a different organism or a different plant species than the plant cell into which the nucleic acid molecule is introduced or can be a nucleic acid molecule derived from the same plant species as the plant cell into which it is introduced. The exogenous nucleic acid may or may not be integrated in the plant cell's genome. When said exogenous nucleic acid/gene is not integrated, transient expression of the nucleic acid/gene occurs in the plant cell.

Non-limiting examples of methods of introducing genetic modifications in a plant cell can be transposon insertion mutagenesis, T-DNA insertion mutagenesis, T-DNA activation tagging, chemically or radio-induced mutagenesis, TILLING (Targeted Induced Local Lesions In Genomes), site-directed mutagenesis, directed evolution, homologous recombination, introducing and expressing in a plant a nucleic acid encoding an SD1 protein, a DW1 protein, or a DW3 protein, or an element which reduces expression of an SD1 protein, a DW1 protein, or a DW3 protein, introducing a programmable nucleotide modification system such as a CRISPR/Cas system, or any combination thereof.

In some aspects, methods of introducing a nucleic acid modification of the instant disclosure comprise using TILLING. Methods for TILLING are well known in the art and include McCallum et al. (2000) Nat. Biotechnol. 18: 455-457; reviewed by Stemple (2004) Nat. Rev. Genet. 5(2): 145-50, the disclosures of all of which are incorporated herein in their entirety. In short, TILLING is a mutagenesis technology useful to generate and/or identify, and to eventually isolate, mutagenized plants. TILLING also allows selection of plants carrying such mutant plants. TILLING combines high-density mutagenesis with high-throughput screening methods. The steps typically followed in TILLING are: (a) EMS mutagenesis; (b) DNA preparation and pooling of individuals; (c) PCR amplification of a region of interest; (d) denaturation and annealing to allow formation of heteroduplexes; (e) DHPLC, where the presence of a heteroduplex in a pool is detected as an extra peak in the chromatogram; (f) identification of the mutant individual; and (g) sequencing of the mutant PCR product.

Populations or libraries of plants comprising genetic modifications can also be used in a method of the instant disclosure. When populations of plants comprising genetic modifications are used, the method can comprise the identification of a plant in the population comprising a nucleic acid modification that result in a reduction in two or more plant hormone activities selected from auxin transport activity, gibberellic acid (GA) biosynthesis activity, and brassinosteroid (BR) signaling activity. Non-limiting examples of populations of plants comprising genetic modifications include TILLING populations, SNP populations, populations of plants comprising naturally-occurring variations, or any combination thereof. Methods of screening populations of populations of plants comprising genetic modifications to identify are known in the art.

In some aspects, methods of introducing a nucleic acid modification of the instant disclosure comprise using a programmable nucleotide modification system to generate the genetically modified plant. The methods can comprise introducing programmable nucleotide modification system or introducing nucleic acid constructs encoding the components of the programmable nucleotide modification system. Programmable nucleotide modification system can be as described in Section II herein above, and nucleic acid constructs encoding components of the programmable nucleotide modification systems can be as described in Section III herein above.

The programmable nucleotide modification system introduces a nucleic acid modification into the nucleic acid sequence, wherein the nucleic acid sequence encodes an SD1 protein, a DW1 protein, or a DW3 protein. The plant or plant cell is then grown under conditions whereby the nucleic acid expression construct expresses the programmable nucleic acid modification system in the plant or plant cell. Expressing the programmable nucleic acid modification system or expressing the polypeptide or polynucleotide introduces a nucleic acid modification of the nucleic acid sequence encoding the polypeptide or polynucleotide, thereby modifying the expression of the polypeptide or polynucleotide in the plant.

(b) Introduction into the Cell

The method comprises introducing a nucleic acid construct expressing an engineered protein into a cell of interest. As explained above, an engineered protein can be encoded on more than one nucleic acid sequence. Accordingly, a method of the instant disclosure comprises introducing more than one nucleic acid construct into the cell.

The one or more nucleic acid constructs described above may be introduced into the cell by a variety of means. Suitable delivery means include microinjection, electroporation, sonoporation, biolistics, calcium phosphate-mediated transfection, cationic transfection, liposomes and other lipids, dendrimer transfection, heat shock transfection, nucleofection transfection, gene gun delivery, dip transformation, supercharged proteins, cell-penetrating peptides, viral vectors, magnetofection, lipofection, impalefection, optical transfection, Agrobacterium tumefaciens mediated foreign gene transformation, proprietary agent-enhanced uptake of nucleic acids, and delivery via liposomes, immunoliposomes, virosomes, or artificial virions. The choice of means of introducing the system into a cell can and will vary depending on the cell, or the system or nucleic acid nucleic acid constructs encoding the system, among other variables.

### (c) Culturing a Cell

The method further comprises culturing a cell under conditions suitable for expressing the engineered protein. Methods of culturing cells are known in the art. In some aspects, the cell is from an animal, fungi, oomycete or prokaryote. In some aspects, the cell is a plant cell, plant, or plant part. When the cell is in tissue ex vivo, or in vivo within a plant or within a plant part, the plant part and/or plant may also be maintained under appropriate conditions for insertion of the donor polynucleotide. In general, the plant, plant part, or plant cell is maintained under conditions appropriate for cell growth and/or maintenance. Those of skill in the art appreciate that methods for culturing plant cells are known in the art and may and will vary depending on the cell type. Routine optimization may be used, in all cases, to determine the best techniques for a particular cell type. See for example, in Santiago et al. (2008) PNAS 105:5809-5814; Moehle et al. (2007) PNAS 104:3055-3060; Urnov et al. (2005) Nature 435:646-651; Lombardo et al. (2007) Nat. Biotechnology 25:1298-1306; and Taylor et al. (2012) Tropical Plant Biology 5:127-139.

### V. Kits

A further aspect of the present disclosure provides kits for improving the yield of an Eragrostis plant. The kits comprise one or more genetically modified lodging resistant Eragrostis plant comprising two or more nucleic acid modifications that result in a reduction of two or more hormone activities selected from auxin transport activity, gibberellic acid (GA) biosynthesis activity, and brassinosteroid (BR) signaling activity; one or more plants comprising the one or more nucleic acid constructs; or any combination thereof. The genetically modified plant can be as described in Section I herein above, the programmable nucleotide modification system can be as described in Section II herein above, the one or more nucleic acid constructs encoding the components of the programmable nucleotide modification system can be as described in Section III herein above.

The kits may further comprise transfection reagents, cell growth media, selection media, in vitro transcription reagents, nucleic acid purification reagents, protein purification reagents, buffers, and the like. The kits provided herein generally include instructions for carrying out the methods detailed below. Instructions included in the kits may be affixed to packaging material or may be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred aspects(s) thereof, the articles “a”, “an”, “the” and “said” are intended to mean that there are one or more of the elements. The terms “comprising”, “including” and “having” are intended to be inclusive and mean that there may be additional elements other than the listed elements.

A “genetically modified” plant refers to a plant in which the nuclear, organellar or extrachromosomal nucleic acid sequences of a cell has been modified, i.e., the cell contains at least one nucleic acid sequence that has been engineered to contain an insertion of at least one nucleotide, a deletion of at least one nucleotide, and/or a substitution of at least one nucleotide.

As used herein, the term “gene” refers to a DNA region (including exons and introns) encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

As used herein, the term “engineered” when applied to a targeting protein refers to targeting proteins modified to specifically recognize and bind to a nucleic acid sequence at or near a target nucleic acid locus. A “genetically modified” plant refers to a cell in which the nuclear, organellar or extrachromosomal nucleic acid sequences of a cell have been modified, i.e., the cell contains at least one nucleic acid sequence that has been engineered to contain an insertion of at least one nucleotide, a deletion of at least one nucleotide, and/or a substitution of at least one nucleotide.

The term “nucleic acid modification” refers to processes by which a specific nucleic acid sequence in a polynucleotide is changed such that the nucleic acid sequence is modified. The nucleic acid sequence may be modified to comprise an insertion of at least one nucleotide, a deletion of at least one nucleotide, and/or a substitution of at least one nucleotide. The modified nucleic acid sequence is inactivated such that no product is made. Alternatively, the nucleic acid sequence may be modified such that an altered product is made.

As used herein, “protein expression” includes but is not limited to one or more of the following: transcription of a gene into precursor mRNA; splicing and other processing of the precursor mRNA to produce mature mRNA; mRNA stability; translation of the mature mRNA into protein (including codon usage and tRNA availability); production of a mutant protein comprising a mutation that modifies the activity of the protein, including the calcium channel activity; and glycosylation and/or other modifications of the translation product, if required for proper expression and function. The term “heterologous” refers to an entity that is not native to the cell or species of interest.

The terms “nucleic acid” and “polynucleotide” refer to a deoxyribonucleotide or ribonucleotide polymer, in linear or circular conformation. For the purposes of the present disclosure, these terms are not to be construed as limiting with respect to the length of a polymer. The terms may encompass known analogs of natural nucleotides, as well as nucleotides that are modified in the base, sugar and/or phosphate moieties. In general, an analog of a particular nucleotide has the same base-pairing specificity, i.e., an analog of A will base-pair with T. The nucleotides of a nucleic acid or polynucleotide may be linked by phosphodiester, phosphothioate, phosphoramidite, phosphorodiamidate bonds, or combinations thereof.

The term “nucleotide” refers to deoxyribonucleotides or ribonucleotides. The nucleotides may be standard nucleotides (i.e., adenosine, guanosine, cytidine, thymidine, and uridine) or nucleotide analogs. A nucleotide analog refers to a nucleotide having a modified purine or pyrimidine base or a modified ribose moiety. A nucleotide analog may be a naturally occurring nucleotide (e.g., inosine) or a non-naturally occurring nucleotide. Non-limiting examples of modifications on the sugar or base moieties of a nucleotide include the addition (or removal) of acetyl groups, amino groups, carboxyl groups, carboxymethyl groups, hydroxyl groups, methyl groups, phosphoryl groups, and thiol groups, as well as the substitution of the carbon and nitrogen atoms of the bases with other atoms (e.g., 7-deaza purines). Nucleotide analogs also include dideoxy nucleotides, 2′-O-methyl nucleotides, locked nucleic acids (LNA), peptide nucleic acids (PNA), and morpholinos.

The terms “polypeptide” and “protein” are used interchangeably to refer to a polymer of amino acid residues.

As used herein, the terms “target site”, “target sequence”, or “nucleic acid locus” refer to a nucleic acid sequence that defines a portion of a nucleic acid sequence to be modified or edited and to which a homologous recombination composition is engineered to target.

The terms “upstream” and “downstream” refer to locations in a nucleic acid sequence relative to a fixed position. Upstream refers to the region that is 5′ (i.e., near the 5′ end of the strand) to the position, and downstream refers to the region that is 3′ (i.e., near the 3′ end of the strand) to the position.

The term “allele” as used herein refers to one of two or more different nucleotide sequences that occur at a specific locus.

“Backcrossing” refers to the process whereby hybrid progeny are repeatedly crossed back to one of the parents. In a backcrossing scheme, the “donor” parent refers to the parental plant with the desired gene or locus to be introgressed. The “recipient” parent (used one or more times) or “recurrent” parent (used two or more times) refers to the parental plant into which the gene or locus is being introgressed. For example, see Ragot, M. et al. (1995) Marker-assisted backcrossing: a practical example, in Techniques et Utilisations des Marqueurs Moleculaires Les Colloques, Vol. 72, pp. 45-56, and Openshaw et al., (1994) Marker-assisted Selection in Backcross Breeding, Analysis of Molecular marker Data, pp. 41-43. The initial cross gives rise to the F1 generation: the term “BC1” then refers to the second use of the recurrent parent, “BC2” refers to the third use of the recurrent parent, and so on.

The term “crossed” or “cross” means the fusion of gametes via pollination to produce progeny (e.g., cells, seeds or plants). The term encompasses both sexual crosses (the pollination of one plant by another) and selfing (self-pollination, e.g., when the pollen and ovule are from the same plant). The term “crossing” refers to the act of fusing gametes via pollination to produce progeny.

As used herein, an “elite line” is any line that has resulted from breeding and selection for superior agronomic performance.

A “favorable allele” is the allele at a particular locus that confers, or contributes to, a desirable phenotype, e.g., increased GS tolerance, or alternatively, is an allele that allows the identification of plants with decreased GS tolerance that can be removed from a breeding program or planting (“counterselection”). A favorable allele of a marker is a marker allele that segregates with the favorable phenotype, or alternatively, segregates with the unfavorable plant phenotype, therefore providing the benefit of identifying plants.

“Genome” refers to the total DNA, or the entire set of genes, carried by a chromosome or chromosome set.

The terms “phenotype”, or “phenotypic trait” or “trait” refer to one or more traits of an organism. The phenotype can be observable to the naked eye, or by any other means of evaluation known in the art, e.g., microscopy, biochemical analysis, or an electromechanical assay. In some cases, a phenotype is directly controlled by a single gene or genetic locus, i.e., a “single gene trait”. In other cases, a phenotype is the result of several genes.

The term “genotype” is the genetic constitution of an individual (or group of individuals) at one or more genetic loci, as contrasted with the observable trait (the phenotype). Genotype is defined by the allele(s) of one or more known loci that the individual has inherited from its parents. The term genotype can be used to refer to an individual's genetic constitution at a single locus, at multiple led, or, more generally, the term genotype can be used to refer to an individual's genetic make-up for all the genes in its genome.

“Germplasm” refers to genetic material of or from an individual (e.g., a plant), a group of individuals (e.g., a plant line, variety or family), or a clone derived from a line, variety, species, or culture. The germplasm can be part of an organism or cell, or can be separate from the organism or cell. In general, germplasm provides genetic material with a specific molecular makeup that provides a physical foundation for some or all of the hereditary qualities of an organism or cell culture. As used herein, germplasm includes cells, seed or tissues from which new plants may be grown, or plant parts, such as leaves, stems, pollen, or cells, that can be cultured into a whole plant.

A “haplotype” is the genotype of an individual at a plurality of genetic loci, i.e. a combination of alleles. Typically, the genetic loci described by a haplotype are physically and genetically linked, i.e., on the same chromosome segment. The term “haplotype” can refer to sequence, polymorphisms at a particular locus, such as a single marker locus, or sequence polymorphisms at multiple loci along a chromosomal segment in a given genome. The former can also be referred to as “marker haplotypes” or “marker alleles”, while the latter can be referred to as “long-range haplotypes”.

A “heterotic group” comprises a set of genotypes that perform well when crossed with genotypes from a different heterotic group (Hallauer at al. (1998) Corn breeding, p. 463-564. In G. F. Sprague and J. W. Dudley (ed) Corn and corn improvement). Inbred lines are classified into heterotic groups, and are further subdivided into families within a heterotic group, based on several criteria such as pedigree, molecular marker-based associations, and performance in hybrid combinations (Smith at al. (1990) Theor. Appl. Gen. 80:833-840). The two most widely used heterotic groups in the United States are referred to as “Iowa Stiff Stalk Synthetic” (BSSS) and “Lancaster” or “Lancaster Sure Crop” (sometimes referred to as NSS, or Iron-Stiff Stalk).

The term “heterozygous” means a genetic condition wherein different alleles reside at corresponding loci on homologous chromosomes.

The term “homozygous” means a genetic condition wherein identical alleles reside at corresponding loci on homologous chromosomes.

The term “hybrid” means a progeny of mating between at least two genetically dissimilar parents. Without limitation, examples of mating schemes include single crosses, modified single cross, double modified single cross, three-way cross, modified three-way cross, and double cross wherein at least one parent in a modified cross is the progeny of a cross between sister lines.

“Hybridization” or “nucleic acid hybridization” refers to the pairing of complementary RNA and DNA strands as well as the pairing of complementary DNA single strands.

The term “hybridize” means the formation of base pairs between complementary regions of nucleic acid strands.

The term “inbred” means a line that has been bred for genetic homogeneity.

The term “indel” refers to an insertion or deletion, wherein one line may be referred to as having an insertion relative to a second line, or the second line may be referred to as having a deletion relative to the first line.

The term “introgression” or “introgressing” refers to the transmission of a desired allele of a genetic locus from one genetic background to another. For example, introgression of a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele can be, e.g., a selected allele of a marker, a QTL, a transgene, or the like. In any case, offspring comprising the desired allele can be repeatedly backcrossed to a line having a desired genetic background and selected for the desired allele, to result in the allele becoming fixed in a selected genetic background. For example, the GS locus described herein may be introgressed into a recurrent parent that has increased GS tolerance. The recurrent parent line with the introgressed gene or locus then has increased GS tolerance.

A “physical map” of the genome is a map showing the linear order of identifiable landmarks (including genes, markers, etc.) on chromosome DNA. However, in contrast to genetic maps, the distances between landmarks are absolute (for example, measured in base pairs or isolated and overlapping contiguous genetic fragments) and not based on genetic recombination.

A “plant” can be a whole plant, any part thereof, or a cell or tissue culture derived from a plant. Thus, the term “plant” can refer to any of: whole plants, plant components or organs (e.g., leaves, stems, roots, etc.), plant tissues, seeds, plant cells, and/or progeny of the same. A plant cell is a cell of a plant, taken from a plant, or derived through culture from a cell taken from a plant.

A “polymorphism” is a variation in the DNA that is too common to be due merely to new mutation. A polymorphism must have a frequency of at least 1% in a population. A polymorphism can be a single nucleotide polymorphism, or SNP, or an insertion/deletion polymorphism, also referred to herein as an “indel”.

The term “progeny” refers to the offspring generated from a cross.

A “progeny plant” is generated from a cross between two plants.

A “reference sequence” is a defined sequence used as a basis for sequence comparison. The reference sequence is obtained by genotyping a number of lines at the locus, aligning the nucleotide sequences in a sequence alignment program (e.g. Sequencher), and then obtaining the consensus sequence of the alignment.

A “single nucleotide polymorphism (SNP)” is an allelic single nucleotide-A, T, C or G-variation within a DNA sequence representing one locus of at least two individuals of the same species. For example, two sequenced DNA fragments representing the same locus from at least two individuals of the same species, contain a difference in a single nucleotide.

The term “quantitative trait locus (QTL)” means a locus that controls to some degree numerically representable traits that are usually continuously distributed.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences may also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) may be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm may be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP may be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs may be found on the GenBank website. With respect to sequences described herein, the range of desired degrees of sequence identity is approximately 80% to 100% and any integer value therebetween. Typically the percent identities between sequences are at least 70-75%, preferably 80-82%, more preferably 85-90%, even more preferably 92%, still more preferably 95%, and most preferably 98% sequence identity.

As various changes could be made in the above-described cells and methods without departing from the scope of the invention, it is intended that all matter contained in the above description and in the examples given below, shall be interpreted as illustrative and not in a limiting sense.

SEQUENCES

| SEQ. | | | |
|---|---|---|---|
| ID NO. | Sequence | | Info |
| 1 | GGCCCGGACTTCGAGCCAAT | | Sd1A1 and |
| | | | Sd1A2 WT. |
| | | | Target of |
| | | | gRNA1 |
| 2 | GGCCCGGACTTCGA--CAAT | | Sd1A1 and |
| | | | Sd1A2 |
| | | | mutation. |
| | | | Target of |
| | | | gRNA1 |
| 3 | GCGACTTCTTCGAGGACAGC | | Sd1A1 and |
| | | | Sd1A2 WT. |
| | | | Target of |
| | | | gRNA2 |
| 4 | GCGACTTCTTCGAGGACAAGC | | Sd1A1 and |
| | | | Sd1A2 |
| | | | mutation. |
| | | | Target of |
| | | | gRNA2 |
| 5 | GGCCCGGACTTCGAGCCAAT | | Sd1B1 and |
| | | | Sd1B2 WT. |
| | | | Target of |
| | | | gRNA1 |
| 6 | GGCCCGGACTTCGAGCCAAAT | | Sd1B1 and |
| | | | Sd1B2 |
| | | | mutation. |
| | | | Target of |
| | | | gRNA1 |
| 7 | GCGACTTCTTCGAGGACAGC | | Sd1B1 and |
| | | | Sd1B2 WT. |
| | | | Target of |
| | | | gRNA2 |
| 8 | GCGACTTCTTCGAGGACAGC | | Sd1B1 and |
| | | | Sd1B2 |
| | | | mutation. |
| | | | Target of |
| | | | gRNA2 |
| 9 | GTGACACACCATATTCCAGG | | Dw1A1 and Dw |
| | | | 1A2 WT. |
| | | | Target of |
| | | | gRNA |
| 10 | GTGAACACACCATATTCCAGG | | Dw1A1 and Dw |
| | | | 1A2 |
| | | | mutation. |
| | | | Target of |
| | | | gRNA |
| 11 | GTGACACACCATATTCCAGG | | Dw1B1 and Dw |
| | | | 1B2 WT. |
| | | | Target of |
| | | | gRNA |
| 12 | GTGAACACACCATATTCCAGG | | Dw1B1 and |
| | | | Dw1B2 |
| | | | mutation. |
| | | | Target of |
| | | | gRNA |
| 13 | GTTCATGATCGGGCGCACGA | | Dw3A1 and Dw |
| | | | 3A2 WT. |
| | | | Target of |
| | | | gRNA |
| 14 | GTTTCATGATCGGGCGCACGA | | Dw3A1 and Dw |
| | | | 3A2 |
| | | | mutation. |
| | | | Target of |
| | | | gRNA |
| 15 | GTTCATGATCGGGCGCACGA | | Dw3B1 and |
| | | | Dw3B2 WT. |
| | | | Target of |
| | | | gRNA |
| 16 | GTTACATGATCGGGCGCACGA | | Dw3B1 and |
| | | | Dw3B2 |
| | | | mutation. |
| | | | Target of |
| | | | gRNA |
| 17 | MASPVAGPTGLPLSLLAPLLCYKYPTGPDRSPAHSPLAHLMVSQARQEPRN | | GA20 Oxidase |
| | SGISKLVMDAIPAPPLLLRSPAPGIDKIPAPEVWPQDDSRPTSAAALDVPV | | protein |
| | VDVGVLRNGGDAAGLRRAAAQVASACATHGFFQVCGHGVDAALARAALDGA | | (SD1A |
| | SDFFRLPLAEKQRARRVPGTVSGYTSAHADRFACKLPWKETLSFRFHDAGA | | protein) |
| | ASPVVADYFTSTLGPDFEPMGRVYQRYCEEMKELSLTIMELLELSLGVERG | | encoded by |
| | YYRDFFEDSRSIMRCNYYPPCPEPERTLGTGPHCDPTALTILLQDDVGGLE | | Eragrostis |
| | VLVDGEWRPVRPVPGAMVINIGDTFMALSNGRYKSCLHRAVVNRRQERRSL | | tef Sd1A |
| | AFFLCPREDRVVRPPPAAAAPRRYPDFTWADLMRFTQSHYRADTRTLDAFT | | Gene (A |
| | NWLARGPAQQQA | | Genome) |
| 18 | 1 | atggcgtccc ctgtggcagg ccccacaggg ttgcctttgt cgttgcttgc ccctctcctc | Eragrostis |
| | 61 | tgttacaaat accccaccgg tccggacagg tcccctgcac actcacccct cgcacatctc | tef Sd1A |
| | 121 | atggtgtccc aagcacggca agagcctcgc aacagcggca tctcgaagct cgtcatggac | Gene (A |
| | 181 | gccatcccgg cccctcctct cctgctccgc tctccagctc ccggcattga caagatcccc | Genome) |
| | 241 | caagatcccc tgtggccgca ggacgactcg cgcccgacgt cggcggcggc gctggacgtc | Encoding |
| | 301 | cccgtggtgg acgtgggcgt gctccgcaat ggcggcgacg ccgccgggct gcggcgcgcg | GA20 Oxidase |
| | 361 | gcggcgcagg tggcgtcggc gtgcgcgacg cacggcttct tccaggtgtg cgggcacggc | protein |
| | 421 | gtggacgcgg ccctggcgcg cgccgcgctg gacggcgcat ccgacttctt ccggctgccg | (SD1B |
| | 481 | ctggccgaga agcagcgcgc ccggcgcgtg ccaggcaccg tgtccgggta cacgagcgcg | protein) |
| | 541 | cacgccgacc ggttcgcgtg caagctgccc tggaaggaga cgctgtcctt ccgcttccac | |
| | 601 | gacgccggcg ccgcgtcgcc cgtggtcgcc gactacttca ccagcaccct cggcccggac | |
| | 661 | ttcgagccaa tggggtacgt aaccaaccac gttgactggt ttaattaatt actgcatata | |
| | 721 | tagtatccat catctccatg gatatatata tatatatata tatatatata tatatatata | |
| | 781 | tatatatata tatatatctt ggcattgcat cattggacac gtacacacgg acggctgaaa | |
| | 841 | aaactatatg gcaggcgcgt gtaccagagg tactgcgagg agatgaagga gctgtccctg | |
| | 901 | acgatcatgg agctgctgga gctgagcctg ggcgtggagc gcggctacta ccgcgacttc | |
| | 961 | ttcgaggaca gccggtccat catgcggtgc aactactacc cgccgtgccc ggagccggag | |
| | 1021 | cgcacgctgg gcacgggccc gcactgcgac cccaccgcgc tcaccatcct cctccaggac | |
| | 1081 | gacgtcggcg gactcgaggt cctcgtcgac ggcgaatggc gccccgtacg acccgtgccc | |
| | 1141 | ggcgccatgg tcatcaacat cggcgacacg ttcatggtat ggtattgtat tactctgctg | |
| | 1201 | ctcttggctg cctgcttgct tgtcctgccc ctgtttctcc tcctaatgga agtgattgga | |
| | 1261 | cagtcgcaga ttacacacac tcacacgtag ctgcctaggc gtgtcatgtt gcgcaatgta | |
| | 1321 | gacatgcgtt cgagttggtt ccatctcgat cggtgggcta gctcggtagt acaggtctat | |
| | 1381 | caatcggtgg tggcgaacgg cgagccgcct tgatgatttc gccaaagaaa aaaaagaaag | |
| | 1441 | aaagagagag gttgcgatgc agtaggagga caaagagaga gagagagaga gagagagaga | |
| | 1501 | gagaagaggt tggcttccgc cgttggcggc ttgcgagttg tagaggagtg ggggaggccc | |
| | 1561 | ggaggaggag ggggaggata cgatgccgcg agcggtggcg ctttcgccgg gtggacccaa | |
| | 1621 | gcccaggtgc gcgcgctcgt cgtcttccct gttttggggc cgtccgggtc ggtggggcca | |
| | 1681 | tctcatctca tcccgcggcg tgactgactg tgcgctgcac gggccgccgc gctggatcat | |
| | 1741 | acatgggctt ggcgttacca ctggcagctt ctacttggct gtcgctcgtc ggcttctagc | |
| | 1801 | cgtttccacg gcgtttgtcg cggttttccg gtgcacgcgg cgcccgcctc cagctcggcc | |
| | 1861 | gtgcgggtgg cacgacacca tcaccccgtc acccgactca cccgtgcgtg cggcttggct | |
| | 1921 | ccgccacgcg ggccgaaaac aaaagggttc ctgtgccttg acaggtcgtc acgtgttgtg | |
| | 1981 | ttgtgcaaaa gaactccgga ttgagctaat tgttgtttcg ctcactgttg attgcgccgc | |
| | 2041 | cgatcgccgg cagcatctct gacttcagca actgactcct ctgctggttg aacgcaggcg | |
| | 2101 | ctgtcgaacg ggcggtacaa gagctgcctg caccgcgcgg tggtgaaccg gcggcaggag | |
| | 2161 | cggcggtcgc tggccttctt cctgtgcccg cgcgaggacc gcgtcgtgcg ccccccgccg | |
| | 2221 | gccgccgccg cgccgcgccg ctacccggac ttcacgtggg ccgacctcat gcgcttcacg | |
| | 2281 | cagagccact accgcgccga cacacgcacc ctcgacgcat tcaccaactg gctcgcccgc | |
| | 2341 | ggcccggccc agcagcaggc gtag | |
| 19 | MLVFVVARSPPLFSCYKYPTGPDRSPAHSHLAHLMVSQARQEPRSSGIKLA | | GA20 Oxidase |
| | MDAIPAPPLLLRSPAPGIDLPKDKIPAPFVWPPDDSRPTSAAALDVPVVDV | | protein (SD1 |
| | GVLRNGGDPAGLRRAAAQVASACATHGFFQVRGHGVDAALARAALDGASDF | | B protein) |
| | FRLPLAEKQRARRLPGTVSGYTSAHADRFACKLPWKETLSFGFHDGAAASP | | encoded by |
| | AVVVDYFTSTLGPDFEPMGRVYQRYCEEMKDLSLTIMELLELSLGVERGYY | | Eragrostis |
| | RDFFEDSRSIMRCNYYPPCPEPERTLGTGPHCDPTALTILLQDDVGGLEVL | | tef Sd1B |
| | VDGEWRPVRPVPGAMVINIGDTFMALSNGRYKSCLHRAVVNRRQERRSLAF | | Gene (B |
| | FLCPREDRVVRPPTTVAAPRRYPDFTWADFMRFTQSHYRAD | | Genome) |
| | TRTLDAFTTWLARGPPAQQA | | |
| 20 | 1 | atgcttgtgt ttgtcgttgc tcgctcccct cccctgttct cctgttacaa ataccccacc | Eragrostis |
| | 61 | ggcccggaca ggtcccctgc acactcacac ctcgcacatc tcatggtgtc ccaagcacgg | tef Sd1B |
| | 121 | caagagcctc gcagcagcgg catctccaag ctcgccatgg acgccatccc ggccccgcct | Gene (B |
| | 181 | ctcctgctcc gctccccagc tcccggcatt gacctcccca aagacaagat ccccgcgccg | Genome) |
| | 241 | ttcgtgtggc cgccggacga ctcgcggccg acgtcggcgg cggcgctgga cgtccccgtg | Encoding |
| | 301 | gtggacgtgg gcgtgctgcg caatggcggc gaccccgccg ggctgcggcg cgcggcggcg | GA20 Oxidase |
| | 361 | caggtggcgt cggcgtgcgc gacgcacggc ttcttccagg tgcgcgggca cggcgtggac | protein (SD1 |
| | 421 | gccgccctgg cgcgcgcggc gctggacggc gccagcgact tcttccggct gccgctcgcc | B protein) |
| | 481 | gagaagcagc gcgcgcggcg actccccggc accgtgtccg ggtacacgag cgcccacgcc | |
| | 541 | gaccggttcg cgtgcaagct cccgtggaag gagacgctgt ccttcggctt ccacgacggc | |
| | 601 | gccgctgcgt cgccggccgt cgtcgtcgac tacttcacca gcaccctcgg cccggacttc | |
| | 661 | gagccaatgg ggtaacatat aaacactact ggcgggcttg ctgtggatat agtatcttgt | |
| | 721 | cgccattgca ttcgacaccg acactagctt gggacgactg gaaccatcca tcatgtacta | |
| | 781 | cttatatggc aggcgcgtgt accagaggta ctgcgaggag atgaaggacc tgtccctgac | |
| | 841 | gatcatggag ctgcttgagc tgagcctggg cgtggagcgc ggctactacc gcgacttctt | |
| | 901 | cgaggacagc cggtccatca tgcggtgcaa ctactacccg ccgtgcccgg agccggagcg | |
| | 961 | cacgctgggc acgggcccgc actgcgaccc cacggcgctc accatcctgc tccaggacga | |
| | 1021 | cgtcggcgga ctcgaggtcc tcgtcgacgg ggaatggcgc cccgtccgcc ccgtgcccgg | |
| | 1081 | agccatggtc atcaacatcg gcgacacgtt catggtacaa ttcaatttac tctgttcttt | |
| | 1141 | ggctggctgc tagcttgtcc tgccctgttc ccgatgtcct cctagctaat ggaactaatt | |
| | 1201 | ggacagcgct cagattacac acacacgtag gcgtgtcatg ttgcgcaatg tagacatgcg | |
| | 1261 | ttcgagttgg ttccatctcg atccgtgggc tagctcggta gtaggtctac cggtggtggt | |
| | 1321 | gaacggtgag ccgccttgat ggtttcgcga aagaagaaaa aagagagaga gagagagaga | |
| | 1381 | gagagaggta gcgatgcagt aggaggacaa agagagagag agaagaggtt ggcttgtgcc | |
| | 1441 | gttggcggct tgcgagttgt agaggagtgg gggaggaccg gaggaggagg gggatacgat | |
| | 1501 | gccgcgagcg gtggcgcttt cgcctggtgg ccccaagccc aggtgcgcgc gcgctcgttg | |
| | 1561 | tcttccctgt tttgggcccg tccggggccg tctcatccgc ggcgtgactg tgcgccgcac | |
| | 1621 | gggccgccgc gctggagcac aggatcgtca gcttggcgtt acccgctggt atgctctgcg | |
| | 1681 | taattctact tggctgtcgc tcgccgggtt ctagccgttt ccgcggcgtt tggcgcggtt | |
| | 1741 | tcccggtgca cgcggcgccc gcgcgggggc tccagctcgg tgcgggtggc actacgctag | |
| | 1801 | cgtatagtac gcgcgccgca acaaaagggt tcctgtgcct tgacaagcct tcactcacgt | |
| | 1861 | ctagtgtttg cacaaaaggg gggggtttcc ggattgagct gattgttgtt tcgttccagt | |
| | 1921 | gttcactgtt gatctgcatc tctccctgac tgactgactg actgactgac tgacttctct | |
| | 1981 | actggttgtt tgcacactgg caacaggcgc tgtccaacgg gcggtacaag agctgcctgc | |
| | 2041 | accgcgcggt ggtgaaccgg cggcaggagc ggcggtcgct ggccttcttc ctgtgcccgc | |
| | 2101 | gcgaggaccg cgtcgtgcgc ccgccgacca ccgtcgccgc gccgcgccgc tacccggact | |
| | 2161 | tcacgtgggc cgacttcatg cgcttcacgc agagccacta ccgcgccgac acacgcaccc | |
| | 2221 | tcgacgcctt caccacctgg ctcgcccgcg gcccgccggcccagcaggcg tag | |
| 21 | 1 | atggcgtccc ctgtggcagg ccccacaggg ttgcctttgt cgttgcttgc ccctctcctc | Eragrostis |
| | 61 | tgttacaaat accccaccgg tccggacagg tcccctgcac actcacccct cgcacatctc | tef Sd1A |
| | 121 | atggtgtccc aagcacggca agagcctcgc aacagcggca tctcgaagct cgtcatggac | Gene (A |
| | 181 | gccatcccgg cccctcctct cctgctccgc tctccagctc ccggcattga caagatcccc | Genome) |
| | 241 | caagatcccc tgtggccgca ggacgactcg cgcccgacgt cggcggcggc gctggacgtc | comprising |
| | 301 | cccgtggtgg acgtgggcgt gctccgcaat ggcggcgacg ccgccgggct gcggcgcgcg | nucleic acid |
| | 361 | gcggcgcagg tggcgtcggc gtgcgcgacg cacggcttct tccaggtgtg cgggcacggc | modification |
| | 421 | gtggacgcgg ccctggcgcg cgccgcgctg gacggcgcat ccgacttctt ccggctgccg | that results |
| | 481 | ctggccgaga agcagcgcgc ccggcgcgtg ccaggcaccg tgtccgggta cacgagcgcg | in a |
| | 541 | cacgccgacc ggttcgcgtg caagctgccc tggaaggaga cgctgtcctt ccgcttccac | reduction in |
| | 601 | gacgccggcg ccgcgtcgcc cgtggtcgcc gactacttca ccagcaccct cggcccggac | GA |
| | 661 | ttcga--caa tggggtacgt aaccaaccac gttgactggt ttaattaatt actgcatata | biosynthesis |
| | 721 | tagtatccat catctccatg gatatatata tatatatata tatatatata tatatatata | |
| | 781 | tatatatata tatatatctt ggcattgcat cattggacac gtacacacgg acggctgaaa | |
| | 841 | aaactatatg gcaggcgcgt gtaccagagg tactgcgagg agatgaagga gctgtccctg | |
| | 901 | acgatcatgg agctgctgga gctgagcctg ggcgtggagc gcggctacta ccgcgacttc | |
| | 961 | ttcgaggacaa gccggtccat catgcggtgc aactactacc cgccgtgccc ggagccggag | |
| | 1021 | cgcacgctgg gcacgggccc gcactgcgac cccaccgcgc tcaccatcct cctccaggac | |
| | 1081 | gacgtcggcg gactcgaggt cctcgtcgac ggcgaatggc gccccgtacg acccgtgccc | |
| | 1141 | ggcgccatgg tcatcaacat cggcgacacg ttcatggtat ggtattgtat tactctgctg | |
| | 1201 | ctcttggctg cctgcttgct tgtcctgccc ctgtttctcc tcctaatgga agtgattgga | |
| | 1261 | cagtcgcaga ttacacacac tcacacgtag ctgcctaggc gtgtcatgtt gcgcaatgta | |
| | 1321 | gacatgcgtt cgagttggtt ccatctcgat cggtgggcta gctcggtagt acaggtctat | |
| | 1381 | caatcggtgg tggcgaacgg cgagccgcct tgatgatttc gccaaagaaa aaaaagaaag | |
| | 1441 | aaagagagag gttgcgatgc agtaggagga caaagagaga gagagagaga gagagagaga | |
| | 1501 | gagaagaggt tggcttccgc cgttggcggc ttgcgagttg tagaggagtg ggggaggccc | |
| | 1561 | ggaggaggag ggggaggata cgatgccgcg agcggtggcg ctttcgccgg gtggacccaa | |
| | 1621 | gcccaggtgc gcgcgctcgt cgtcttccct gttttggggc cgtccgggtc ggtggggcca | |
| | 1681 | tctcatctca tcccgcggcg tgactgactg tgcgctgcac gggccgccgc gctggatcat | |
| | 1741 | acatgggctt ggcgttacca ctggcagctt ctacttggct gtcgctcgtc ggcttctagc | |
| | 1801 | cgtttccacg gcgtttgtcg cggttttccg gtgcacgcgg cgcccgcctc cagctcggcc | |
| | 1861 | gtgcgggtgg cacgacacca tcaccccgtc acccgactca cccgtgcgtg cggcttggct | |
| | 1921 | ccgccacgcg ggccgaaaac aaaagggttc ctgtgccttg acaggtcgtc acgtgttgtg | |
| | 1981 | ttgtgcaaaa gaactccgga ttgagctaat tgttgtttcg ctcactgttg attgcgccgc | |
| | 2041 | cgatcgccgg cagcatctct gacttcagca actgactcct ctgctggttg aacgcaggcg | |
| | 2101 | ctgtcgaacg ggcggtacaa gagctgcctg caccgcgcgg tggtgaaccg gcggcaggag | |
| | 2161 | cggcggtcgc tggccttctt cctgtgcccg cgcgaggacc gcgtcgtgcg ccccccgccg | |
| | 2221 | gccgccgccg cgccgcgccg ctacccggac ttcacgtggg ccgacctcat gcgcttcacg | |
| | 2281 | cagagccact accgcgccga cacacgcacc ctcgacgcat tcaccaactg gctcgcccgc | |
| | 2341 | ggcccggccc agcagcaggc gtag | |
| 22 | 1 | atgcttgtgt ttgtcgttgc tcgctcccct cccctgttct cctgttacaa ataccccacc | Eragrostis |
| | 61 | ggcccggaca ggtcccctgc acactcacac ctcgcacatc tcatggtgtc ccaagcacgg | tef Sd1B |
| | 121 | caagagcctc gcagcagcgg catctccaag ctcgccatgg acgccatccc ggccccgcct | Gene (B |
| | 181 | ctcctgctcc gctccccagc tcccggcatt gacctcccca aagacaagat ccccgcgccg | Genome) |
| | 241 | ttcgtgtggc cgccggacga ctcgcggccg acgtcggcgg cggcgctgga cgtccccgtg | comprising |
| | 301 | gtggacgtgg gcgtgctgcg caatggcggc gaccccgccg ggctgcggcg cgcggcggcg | nucleic acid |
| | 361 | caggtggcgt cggcgtgcgc gacgcacggc ttcttccagg tgcgcgggca cggcgtggac | modification |
| | 421 | gccgccctgg cgcgcgcggc gctggacggc gccagcgact tcttccggct gccgctcgcc | that results |
| | 481 | gagaagcagc gcgcgcggcg actccccggc accgtgtccg ggtacacgag cgcccacgcc | in a |
| | 541 | gaccggttcg cgtgcaagct cccgtggaag gagacgctgt ccttcggctt ccacgacggc | reduction in |
| | 601 | gccgctgcgt cgccggccgt cgtcgtcgac tacttcacca gcaccctcgg cccggacttc | GA |
| | 661 | gagccaaatgg ggtaacatat aaacactact ggcgggcttg ctgtggatat agtatcttgt | biosynthesis |
| | 721 | cgccattgca ttcgacaccg acactagctt gggacgactg gaaccatcca tcatgtacta | |
| | 781 | cttatatggc aggcgcgtgt accagaggta ctgcgaggag atgaaggacc tgtccctgac | |
| | 841 | gatcatggag ctgcttgagc tgagcctggg cgtggagcgc ggctactacc gcgacttctt | |
| | 901 | cgaggacagc cggtccatca tgcggtgcaa ctactacccg ccgtgcccgg agccggagcg | |
| | 961 | cacgctgggc acgggcccgc actgcgaccc cacggcgctc accatcctgc tccaggacga | |
| | 1021 | cgtcggcgga ctcgaggtcc tcgtcgacgg ggaatggcgc cccgtccgcc ccgtgcccgg | |
| | 1081 | agccatggtc atcaacatcg gcgacacgtt catggtacaa ttcaatttac tctgttcttt | |
| | 1141 | ggctggctgc tagcttgtcc tgccctgttc ccgatgtcct cctagctaat ggaactaatt | |
| | 1201 | ggacagcgct cagattacac acacacgtag gcgtgtcatg ttgcgcaatg tagacatgcg | |
| | 1261 | ttcgagttgg ttccatctcg atccgtgggc tagctcggta gtaggtctac cggtggtggt | |
| | 1321 | gaacggtgag ccgccttgat ggtttcgcga aagaagaaaa aagagagaga gagagagaga | |
| | 1381 | gagagaggta gcgatgcagt aggaggacaa agagagagag agaagaggtt ggcttgtgcc | |
| | 1441 | gttggcggct tgcgagttgt agaggagtgg gggaggaccg gaggaggagg gggatacgat | |
| | 1501 | gccgcgagcg gtggcgcttt cgcctggtgg ccccaagccc aggtgcgcgc gcgctcgttg | |
| | 1561 | tcttccctgt tttgggcccg tccggggccg tctcatccgc ggcgtgactg tgcgccgcac | |
| | 1621 | gggccgccgc gctggagcac aggatcgtca gcttggcgtt acccgctggt atgctctgcg | |
| | 1681 | taattctact tggctgtcgc tcgccgggtt ctagccgttt ccgcggcgtt tggcgcggtt | |
| | 1741 | tcccggtgca cgcggcgccc gcgcgggggc tccagctcgg tgcgggtggc actacgctag | |
| | 1801 | cgtatagtac gcgcgccgca acaaaagggt tcctgtgcct tgacaagcct tcactcacgt | |
| | 1861 | ctagtgtttg cacaaaaggg gggggtttcc ggattgagct gattgttgtt tcgttccagt | |
| | 1921 | gttcactgtt gatctgcatc tctccctgac tgactgactg actgactgac tgacttctct | |
| | 1981 | actggttgtt tgcacactgg caacaggcgc tgtccaacgg gcggtacaag agctgcctgc | |
| | 2041 | accgcgcggt ggtgaaccgg cggcaggagc ggcggtcgct ggccttcttc ctgtgcccgc | |
| | 2101 | gcgaggaccg cgtcgtgcgc ccgccgacca ccgtcgccgc gccgcgccgc tacccggact | |
| | 2161 | tcacgtgggc cgacttcatg cgcttcacgc agagccacta ccgcgccgac acacgcaccc | |
| | 2221 | tcgacgcctt caccacctgg ctcgcccgcg gcccgccggc ccagcaggcg tag | |
| 23 | MASAGSSSGGAGGGSSIRAANGAAAISAAATAVGSADARFHSHPPQQDRRS | | Protein |
| | RWAGCLSGLSCFGSQKGGKRIVPAARTPDGNGSSARGNGHQSGSNSNQNVP | | involved in |
| | LNLSLLAPPSSPASFSNSALPSTVQSPNNFLSISANSPGGPTSNMFAVGPY | | brassin- |
| | ANEPQLVSPPVFSTYTTEPSTAPLTPPPELAHATTPSSPDVPYARELSSfM | | osteroid |
| | DIKTASKEHNMAFLSTTYSGGSGLQASYPLYPESPCSSLISPASATPRTGL | | signaling |
| | SSPIPEQEVPTAHWKTSRSACDTPYSRASPIPEQEATAQWKTSRSACDTPY | | (DW1A |
| | ARASPSNIFGLDSSASRNYLLDGNFFRPAASAQFYLDQAQQTYPYNGGRRS | | protein) |
| | VSRDKQDADEVEAYRASFGFSADEIMQTQSYVEIPDALDESFSISPFGNNA | | encoded by |
| | PATEVSPFNDPPNEVQKAEKSSPKKTADQISNGSPHRVLHIDIFKGTKGGH | | Eragrostis |
| | PSEYEGIVKDGHPFRKTRDEISLKPIEVRKKSPPGHSCSDAEIEYRRARSL | | tef Dw1A |
| | REANGVPSWRSTLSRQLQ | | Gene |
| | | | (Chromosome |
| | | | 4A) |
| 24 | | | Eragrostis |
| | | | tef Dw1A |
| | | | Gene |
| | | | (Chromosome |
| | | | 4A) Encoding |
| | | | protein |
| | | | involved in |
| | | | brassin- |
| | | | osteroid |
| | | | signaling |
| | | | (DW1A |
| | | | protein) |
| 25 | MASAGSSSGGAGGGSSIRAANGAAAISAAATAVGSADARFHSHPPQQDRRS | | Protein |
| | RWAGCFSGLSCFGSQKGGKRIVPAARTPDGNGSSARGNGHQSGSNSNQNVP | | involved in |
| | LNLSLLAPPSSPASFSNSALPSTVQSPNNFLSISANSPGGPTSNMFAVGPY | | brassin- |
| | ANEPQLVSPPVFSTYTTEPSTAPLTPPPELAHATTPSSPDVPYARFLSSSM | | osteroid |
| | DIKTASKEHNMAFLSTTYSGGSGLQASYPLYPESPCSSLISPASATPRTGL | | signaling |
| | SSPIPEQEVPTAHWKTSRSACDTPYSRASPIPEQEATAQWKTSRSACDTPY | | (DW1B |
| | ARASPSNIFGLDSSASRNYLLDGNFFRPAASAQFYLDQAQQTYPYNGGRRS | | protein) |
| | VSRDKQDADEVEAYRASFGFSADEIMQTQSYVEIPDALDESESISPFGNNA | | encoded by |
| | PATEVSPFNDLPNEVQKAEKSSPKKSADQISNGSPHRVLHVDIFKGTKGGH | | Eragrostis |
| | PFEYEGIVKDGHPFRKTRDEISLKPIEVRKKSPPGHSCSDAEIEYRRARSL | | tef Dw1B |
| | REANGVPSWRSTLSRQLQ | | Gene |
| | | | (Chromosome |
| | | | 4B) |
| 26 | 1 | atggcttcag ctgggagcag cagtggaggt gcaggtggag gcagcagcat cagggcggcg | Eragrostis |
| | 61 | aatggcgcag ctgctatcag tgcggccgcg acggcagtag gctcagccga cgccagattc | tef Dw1B |
| | 121 | cactcccacc caccacaaca ggacagggta tatacactac acgacttctt gttccatggc | Gene |
| | 181 | gcatgcaatt gctcatgctt tggctgcagc ttttaccctt cttgctttag atgtatggat | (Chromosome |
| | 241 | gagtaccagc aatttccagt aggccttgac gacctagatt gtcacaggct ttgcgtactt | 4B) Encoding |
| | 301 | agtacgaatg gtactgccca gtggtggttg cagcttgcaa cccccggcat ctttaccccc | protein |
| | 361 | aataattagg cttcagtact gtctgcttgt ttgcttctac tgaataattt gttcggctga | involved in |
| | 421 | atttttcgtt ttgctgtcga tattgattgc acgccatttg cactccctct atgtactctt | brassin- |
| | 481 | accagatgac tgtttgtact cttaccataa cactcctcca gagatcctag atgctgtata | oosterid |
| | 541 | actgttgatt tgaccttgga tacacttcca cacgttgtgt cagttgatgt catgagcaaa | signaling |
| | 601 | ctaactatgc tgggctgaaa tcttcttgca tgataaatct ttttccatat gaaaattttc | (DW1B |
| | 661 | agatggctgt aaaattcttg gtggtgtagc tattattgtc aacaaatttc ttatattctt | protein) |
| | 721 | tagtaaactt gttttttatg cttacccgtt atagtaagtt tggcccagtt tcccttccca | |
| | 781 | tgattttaat gaataaatca ctttttctgt ttgtagcgag tatagattaa tactctgagg | |
| | 841 | aaatttcgtt gttgaacatt tagtcataca tttcctccct accaacagct atgtacttga | |
| | 901 | tactaatatg tgcatctctg atctaaacag cggagtagat gggctggctg cttttcgggc | |
| | 961 | ctttcatgtt tcggatctca gaagggcggg aagaggattg ttccggccgc acgtactcct | |
| | 1021 | gatgggaatg gatcaagtgc tcgtggaaat ggtcatcagt ctggttctaa ttcaaaccaa | |
| | 1081 | aatgtgcctt taaatctgtc gcttctggct ccaccatcgt caccggcatc cttctcaaat | |
| | 1141 | tctgcacttc cttcaactgt tcagtcgcca aataactttc tgtcaatctc agcaaattct | |
| | 1201 | cctggtggtc cgacatctaa tatgtttgct gttgggccat atgctaatga acctcagctt | |
| | 1261 | gtctcgcctc ctgtcttctc aacttacaca actgagcctt ccacagcacc attgacccct | |
| | 1321 | ccacctgaac tagctcatgc aaccactccc tcgtccccag atgttccata tgcccggttt | |
| | 1381 | ctttcttctt ctatggatat caaaactgct agtaaggagc ataacatggc tttcttatca | |
| | 1441 | acaacctatt ctggtggttc aggactccag gcatcatacc ctctttaccc tgagagccct | |
| | 1501 | tgtagcagcc tcatatcacc tgcttctgca actccgagga ctggtctttc ctcaccaata | |
| | 1561 | cctgaacaag aggtccctac agcccattgg aagacttcta ggtctgcctg tgacacacca | |
| | 1621 | tattccaggg cttcacccat ccctgagcaa gaagccactg cgcagtggaa gacttctaga | |
| | 1681 | tcagcatgtg atacacctta tgccagagct tcaccatcaa acatctttgg gctggattca | |
| | 1741 | tctgcttcta gaaactattt gttagatggc aacttcttcc ggccagctgc ttctgctcaa | |
| | 1801 | ttctacctgg accaggctca gcagacatat ccatataatg gtgggaggcg tagtgtctca | |
| | 1861 | cgggacaagc aagacgcaga tgaagttgaa gcttacagag cttcgtttgg ctttagtgca | |
| | 1921 | gatgaaatca tgcaaactca atcttatgtg gagataccag atgcacttga tgaatcattc | |
| | 1981 | agtatatcac catttggaaa taatgctcct gctactgagg tgtccccatt taatgatctg | |
| | 2041 | cccaatgagg ttcagaaggc tgagaagtca agtccaaaga aatcagcaga tcagatttcc | |
| | 2101 | aatggttctc cacatagagt gctgcacgtt gacatattca agggtaaatg tccatacttt | |
| | 2161 | ttgcatcaac cattcagttg taatctaggc gtagtagaat tactcctttt gttatccttg | |
| | 2221 | ctatctactg cacctaactg gtaacttcca atgctaataa cctgaactct gggccattgg | |
| | 2281 | tattgtgttt ctgtttacgt aaatgcttat catgcttatg tgctcacatt tcaggaacaa | |
| | 2341 | aaggaggaca tccgtttgag tacgagggga ttgtgaaaga tggccatcct ttcagaaaga | |
| | 2401 | caagggatga gatatctcta aaacccatag aagtaaggaa gaaatctcca cctggtcatt | |
| | 2461 | catgctcaga tgctgaaatc gagtacagaa gggcaaggag tctgagggaa gccaacggtg | |
| | 2521 | tcccctcgtg gcgcagcacg ttgtcgagac agctgcagtg a | |
| 27 | 1 | atggcttcag ctgggagcag cagtggaggt gcaggtggag gcagcagcat cagggggcg | Eragrostis |
| | 61 | aatggcgcag ctgccatcag tgcggccgcg acggcagtag gctcagccga cgccagattc | tef Dw1A |
| | 121 | cactcccacc caccacagca ggacagggta tatacactac acgacttctt gttccatggc | Gene (A |
| | 181 | gcatgcaatt gctcatgctt tagctgcagc ttttaccctt cttgctttag atgtgtggat | Genome) |
| | 241 | gagtaccagc aatttctagt aggccttgac gacctagatt gtcacaggct ttgcgtactt | comprising |
| | 301 | agtacgaatg gtactgccca gtggtggttg cagcttgcaa ccccggcatc tttaccccca | nucleic acid |
| | 361 | ataattaggc ttcagtactg tctgcttgtt tgcttctagt actgaatagt ttgttcagct | modification |
| | 421 | gaattttccg ttttgctgtt gatatttgat tattacgcga tttgcactcc ctctatgttt | that results |
| | 481 | ttccccccgg atcctagatg actgtactct taccataaca ctcgtccaga gacctatatg | in a |
| | 541 | ttgtgtaact gttgatttga ccttgcatac acttccacac gtcgtgtcag ttgatgtcat | reduction in |
| | 601 | gagcaaacta actatgctgg gttgattcct tcttgcatga tggatctttt tccaaatgaa | brassin- |
| | 661 | ttttcagatt cctgtaaaat gcttggtggt gtagctatta atgtcaacac atttcttata | osteroid (BR) |
| | 721 | ttcttaagta aaattggttt agattaatac tccaaggaaa tttcgttgtt gaacaactag | signaling |
| | 781 | tcatacattt tttcccctac caacagctat gtacttgata ctaatatgtg catctctgat | activity |
| | 841 | ctaaacagcg gagtagatgg gctggctgct tgtcgggtct ttcatgtttc ggatctcaga | |
| | 901 | agggcgggaa gaggattgtt cctgccgcac gtactcctga tgggaatgga tcaagtgctc | |
| | 961 | gtggaaatgg tcatcagtcc ggttctaatt caaaccaaaa tgtgccttta aatctgtctc | |
| | 1021 | ttctggctcc accatcctca ccggcatcct tctcaaattc tgcacttcct tcaactgttc | |
| | 1081 | aatcgcctaa taactttctg tcaatctcag caaattctcc tggtggtcca acatctaata | |
| | 1141 | tgtttgctgt tgggccatat gctaatgaac ctcagcttgt ctcgcctcct gtcttctcaa | |
| | 1201 | cttacacaac tgagccttcc acagcaccat tgacccctcc acctgaacta gctcatgcaa | |
| | 1261 | ccactccctc atctccagat gttccatacg ctcggtttct ttcttctttt atggatatca | |
| | 1321 | aaactgctag taaggagcat aacatggctt tcttatcaac aacatattct ggtggttcag | |
| | 1381 | gactccaggc atcataccct ctttaccctg agagcccttg tagcagcctc atatcacctg | |
| | 1441 | cttctgctac tccgaggact ggtctttcct caccaatacc tgaacaagag gtccctactg | |
| | 1501 | cccattggaa gacttctagg tctgcctgtga acacaccata ttccagggct tcacccatcc | |
| | 1561 | ctgagcaaga agccactgcg cagtggaaga cttctagatc ggcatgtgat acgccttatg | |
| | 1621 | ccagggcttc accatcaaac atctttgggc tggactcatc tgcttctaga aactatttgt | |
| | 1681 | tagatggcaa cttcttccgg ccagctgctt ctgctcaatt ctacctggac caggctcagc | |
| | 1741 | agacatatcc atataatggt gggaggcgta gtgtctcacg ggacaagcaa gatgcagatg | |
| | 1801 | aagttgaagc ttacagagct tcgtttggct ttagtgcaga tgaaatcatg caaactcaat | |
| | 1861 | cttatgtgga gataccagat gcacttgatg aatcattcag tatatcgcca tttggaaata | |
| | 1921 | atgctcctgc tactgaggtg tccccattta atgatccacc caacgaggtt cagaaggctg | |
| | 1981 | agaagtcaag tccaaagaaa acggcagatc agatttccaa tggctctcca catagagtgc | |
| | 2041 | tgcacattga catattcaag ggtaaatgtc catacttttt gcaccaacca ttcagttgta | |
| | 2101 | atctaggcat agtagaatta ctcttgttta tccttgctat ctactgcacc taacaaagct | |
| | 2161 | ttggtaactt tcaatgctaa taacctgaac tctgggccat ttgtcatcag cacttattgg | |
| | 2221 | tattgtgttt ctatttatgt aaatgcttat tatgcttatg tgctcgcatt tcaggaacaa | |
| | 2281 | aaggaggaca tccgtctgag tacgagggta ttgtgaaaga tggccatcct ttcagaaaga | |
| | 2341 | caagggacga aatatctcta aaacccatag aagtaaggaa gaaatctcca cctgggcatt | |
| | 2401 | catgctcaga tgctgaaatt gagtacagaa gggcaaggag tctgagggaa gccaatggtg | |
| | 2461 | tcccctcgtg gcgcagcacg ttgtcaagac agctgcagtg a | |
| 28 | 1 | atggcttcag ctgggagcag cagtggaggt gcaggtggag gcagcagcat cagggggcg | Eragrostis |
| | 61 | aatggcgcag ctgctatcag tgcggccgcg acggcagtag gctcagccga cgccagattc | tef Dw1B |
| | 121 | cactcccacc caccacaaca ggacagggta tatacactac acgacttctt gttccatggc | Gene (B |
| | 181 | gcatgcaatt gctcatgctt tggctgcagc ttttaccctt cttgctttag atgtatggat | Genome) |
| | 241 | gagtaccagc aatttccagt aggccttgac gacctagatt gtcacaggct ttgcgtactt | comprising |
| | 301 | agtacgaatg gtactgccca gtggtggttg cagcttgcaa cccccggcat ctttaccccc | nucleic acid |
| | 361 | aataattagg cttcagtact gtctgcttgt ttgcttctac tgaataattt gttcggctga | modification |
| | 421 | atttttcgtt ttgctgtcga tattgattgc acgccatttg cactccctct atgtactctt | that results |
| | 481 | accagatgac tgtttgtact cttaccataa cactcctcca gagatcctag atgctgtata | in a |
| | 541 | actgttgatt tgaccttgga tacacttcca cacgttgtgt cagttgatgt catgagcaaa | reduction in |
| | 601 | ctaactatgc tgggctgaaa tcttcttgca tgataaatct ttttccatat gaaaattttc | brassin- |
| | 661 | agatggctgt aaaattcttg gtggtgtagc tattattgtc aacaaatttc ttatattctt | osteroid (BR) |
| | 721 | tagtaaactt gttttttatg cttacccgtt atagtaagtt tggcccagtt tcccttccca | signaling |
| | 781 | tgattttaat gaataaatca ctttttctgt ttgtagcgag tatagattaa tactctgagg | activity |
| | 841 | aaatttcgtt gttgaacatt tagtcataca tttcctccct accaacagct atgtacttga | |
| | 901 | tactaatatg tgcatctctg atctaaacag cggagtagat gggctggctg cttttcgggc | |
| | 961 | ctttcatgtt tcggatctca gaagggcggg aagaggattg ttccggccgc acgtactcct | |
| | 1021 | gatgggaatg gatcaagtgc tcgtggaaat ggtcatcagt ctggttctaa ttcaaaccaa | |
| | 1081 | aatgtgcctt taaatctgtc gcttctggct ccaccatcgt caccggcatc cttctcaaat | |
| | 1141 | tctgcacttc cttcaactgt tcagtcgcca aataactttc tgtcaatctc agcaaattct | |
| | 1201 | cctggtggtc cgacatctaa tatgtttgct gttgggccat atgctaatga acctcagctt | |
| | 1261 | gtctcgcctc ctgtcttctc aacttacaca actgagcctt ccacagcacc attgacccct | |
| | 1321 | ccacctgaac tagctcatgc aaccactccc tcgtccccag atgttccata tgcccggttt | |
| | 1381 | ctttcttctt ctatggatat caaaactgct agtaaggagc ataacatggc tttcttatca | |
| | 1441 | acaacctatt ctggtggttc aggactccag gcatcatacc ctctttaccc tgagagccct | |
| | 1501 | tgtagcagcc tcatatcacc tgcttctgca actccgagga ctggtctttc ctcaccaata | |
| | 1561 | cctgaacaag aggtccctac agcccattgg aagacttcta ggtctgcctg tgaacacacca | |
| | 1621 | tattccaggg cttcacccat ccctgagcaa gaagccactg cgcagtggaa gacttctaga | |
| | 1681 | tcagcatgtg atacacctta tgccagagct tcaccatcaa acatctttgg gctggattca | |
| | 1741 | tctgcttcta gaaactattt gttagatggc aacttcttcc ggccagctgc ttctgctcaa | |
| | 1801 | ttctacctgg accaggctca gcagacatat ccatataatg gtgggaggcg tagtgtctca | |
| | 1861 | cgggacaagc aagacgcaga tgaagttgaa gcttacagag cttcgtttgg ctttagtgca | |
| | 1921 | gatgaaatca tgcaaactca atcttatgtg gagataccag atgcacttga tgaatcattc | |
| | 1981 | agtatatcac catttggaaa taatgctcct gctactgagg tgtccccatt taatgatctg | |
| | 2041 | cccaatgagg ttcagaaggc tgagaagtca agtccaaaga aatcagcaga tcagatttcc | |
| | 2101 | aatggttctc cacatagagt gctgcacgtt gacatattca agggtaaatg tccatacttt | |
| | 2161 | ttgcatcaac cattcagttg taatctaggc gtagtagaat tactcctttt gttatccttg | |
| | 2221 | ctatctactg cacctaactg gtaacttcca atgctaataa cctgaactct gggccattgg | |
| | 2281 | tattgtgttt ctgtttacgt aaatgcttat catgcttatg tgctcacatt tcaggaacaa | |
| | 2341 | aaggaggaca tccgtttgag tacgagggga ttgtgaaaga tggccatcct ttcagaaaga | |
| | 2401 | caagggatga gatatctcta aaacccatag aagtaaggaa gaaatctcca cctggtcatt | |
| | 2461 | catgctcaga tgctgaaatc gagtacagaa gggcaaggag tctgagggaa gccaacggtg | |
| | 2521 | tcccctcgtg gcgcagcacg ttgtcgagac agctgcagtg a | |
| 29 | MSSSDPEEIRGRVVVLGADADELARPELEAFHLPSPTLAQEAGSVGGPGPA | | ATP-binding |
| | AAAPVVAPLETELPPNATMPSSSSASSNSNSNEQNKEQENTKKKGVSLAPA | | casscttc |
| | PLGSLFRFADGLDCVLMSVGTLGALVHGCSLPVELRFFADLVDSFGSHADD | | typc B1 |
| | PDTMVRLVAKYALYFLVVGAAIWASSWAEISCWMWTGERQSTRMRIRYLES | | (ABCB1) |
| | ALRQDVSFEDTDVRTSDVIYAINADAVIVQDAISEKLGNLIHYMATFVAGF | | auxin |
| | VVGFTAAWQLALVTLAVVPLIAVIGGLSAAALAKLSSRQDALAEASNIAEQ | | transpcrtcr |
| | AVAQIRTVQAFVGEERAMRAYSLALAAAQRIGYRSGFAKGLGLGGTYFTVF | | protein |
| | CCYALLLWYGGLLVRRHHTNGGLAIATMESVMIGGLALGQSAPSMAAFAKA | | (DW3A) |
| | RVAAAKIFKIIDHKPLSVVVHGDDDVQLPSVTGRVEMRGVDFAYPSRPDVP | | protein) |
| | VLRGFSLTVPPGKTIALVGSSGSGKSTVVSLIERFYDPSAGEILLDGHDLK | | cnccdcd by |
| | SLNRWLRQQIGLVSQEPTLFATSIKENLLLGRDSHSATLAEMEEAARVANA | | Eragrcstis |
| | HSFIIKLPDGYDTQVGERGLQLSGGQKQRIAIARAMLKNPGILLLDEATSA | | tcf Dw3A |
| | LDSESEKLVQEALDREMIGRTTLVIAHRLSTIRKADLVAVLHGGAVSEIGT | | Gcnc (A |
| | HEELMGKGEDGAYARLIRMQEQAAQEVAARRSSARNSVSARNSVSSPIMTR | | gcncmc) |
| | NSSYGRSPYSRRLSDESNADFHYHGGGELPEGNTKKMIHQRVAFRAGASSF | | |
| | LRLAKMNSPEWGYALVGSLGSMVCGSFSAIFAYVLSAVLSVYYAPDPGHMR | | |
| | REIAKYCYLLMGMSSAALVCNTVQHVFWDTVGENLTKRVRERLGAVLRNEM | | |
| | AWFDAEENASARVAARLALDAQNVRSAIGDRISVIVQNSALLLVACTAGFV | | |
| | LQWRLALVLLGVFPLVVAATVLQKMEMKGESGDLEAAHARATQIAGEAVAN | | |
| | LRTVAAFNAEAKIAGLFAGNLRGPLRRCLWKGQVAGCGYGVAQFLLYASYA | | |
| | LGLWYAAWLVKHGVSDESRTIRVEMVLMVSANGAAETLTLAPDFVRGGRAM | | |
| | RSVFETIDRRTEADPDDPDAAPLQLPLLTGELRHVDFCYPSRPEVQVLQDL | | |
| | SLRARAGKTLALVGPSGCGKSSVLALIQRFYEPTSGRVLDGRDARKYNLRA | | |
| | LRRAVAVVPQEPELFAASIHDNIAYGREGGATEAEVLEAATQANAHKFISA | | |
| | LPDGYRTQVGERGVQLSGGQRQRIAVARALVKQAAVLLLDEATSALDAESE | | |
| | RSVQQALDRHAKTRSTTTIVVAHRLATVRNAHTIAVIDEGKVVEQGSHSHL | | |
| | LNHHPDGTYARMLQLQRLTSSTS | | |
| 30 | 1 | atgtctagca gcgacccgga ggagatccgg gggcgcgtgg tcgtcctcgg cgccgatgcg | Eragrostis |
| | 61 | gacgagttgg ctcgccccga gctggaggcc ttccacctcc cgtctccgac actagctcag | tef Dw3A |
| | 121 | gaggcaggat ctgtcggagg cccaggccct gctgctgctg ctcccgtggt ggcgccattg | Gene (A |
| | 181 | gagacggagc tgcctcccaa tgccaccatg ccttcttctt cctccgcctc ctcgaacagc | genome) |
| | 241 | aacagcaatg agcagaacaa ggagcaggag aatacgaaga agaagggggt gtctttggcg | Encoding |
| | 301 | cctgctcctc ttggttcgct gttccgtttc gccgacggtc tggactgcgt tctgatgtcg | ABCB1 |
| | 361 | gtgggcacgt tgggcgcgct tgtccacggt tgctccctcc cggtgttcct ccgcttcttc | protein |
| | 421 | gcggacctcg tcgactcgtt cggctcccac gccgacgacc cggacaccat ggtgcggctg | (DW3A) |
| | 481 | gtggccaagt acgcgctcta cttcctggtg gtgggcgcgg ccatctgggc gtcctcctgg | protein) |
| | 541 | gcggagatct cgtgctggat gtggaccggc gagcggcagt cgacgcggat gcggatccgg | |
| | 601 | tacctggagt cggcgctccg gcaggacgtg tccttcttcg acaccgacgt ccgcacctcc | |
| | 661 | gacgtgatct acgccatcaa cgcggacgcc gtgatcgtgc aggacgccat cagcgagaag | |
| | 721 | ctcggcaacc tcatccacta catggccacc ttcgtggccg ggttcgtggt cgggttcacg | |
| | 781 | gcggcatggc agctggcgct ggtgacgctg gccgtggtgc cgctcatcgc cgtgatcggc | |
| | 841 | ggcctcagcg cggcggcgct ggcgaagctg tcgtcgcgga gccaggacgc gctggcggag | |
| | 901 | gcgagcaaca tcgcggagca ggcggtggcg cagatacgga cggtgcaggc cttcgtcggg | |
| | 961 | gaggagcgcg caatgcgggc ctactcgttg gcgctggccg cggcgcagag gatcggatac | |
| | 1021 | cgcagcggct tcgccaaggg cctgggcctg ggcggcacct acttcaccgt cttctgctgc | |
| | 1081 | tacgcgctgc tgctctggta cggcggcctc ctcgtgcgac gccaccacac caacggcggc | |
| | 1141 | ctcgccatcg ccaccatgtt ctccgtcatg atcgggggcc tcgcgctggg ccagtcggcg | |
| | 1201 | cccagcatgg ccgccttcgc aaaggcgcgc gtcgccgcgg ccaagatctt caaaatcatc | |
| | 1261 | gaccacaagc ccctctccgt cgtcgtccat ggcgacgacg atgtccaact cccttccgtg | |
| | 1321 | acggggcggg tggagatgcg gggcgttgac ttcgcctacc cgtcgcggcc ggacgtcccc | |
| | 1381 | gtcctgcgcg gcttctccct caccgtgccg cccggcaaga ccatcgcgct cgtcgggagc | |
| | 1441 | tcaggctccg ggaagagcac ggtcgtctcc ctcatcgaga ggttctacga cccaagtgca | |
| | 1501 | ggtacacaac tacttaacgt cttagaagtg tctgtcagtc aattgtcttc ttcattcaca | |
| | 1561 | atcgtattca ttgattgaaa gaaaaatatg cttctcaggc gagattctgc ttgacgggca | |
| | 1621 | cgacctcaag tccctcaacc tgcggtggct ccggcagcag atcgggctgg tgagccagga | |
| | 1681 | gccgacgctg ttcgccacca gcatcaagga gaacctgctg ctgggccggg acagccacag | |
| | 1741 | cgccaccctc gccgagatgg aggaggccgc aagggtcgcc aacgcccact cattcatcat | |
| | 1801 | caagctgccc gacggatacg acacccaggt atgtactagc accgttgctt tcctttcaca | |
| | 1861 | ttcctctcct ctcctgagca actagatatg tactagcacc attgctccat ctcaatgcca | |
| | 1921 | ccatcctcaa aaaagaagga aagcttttct ccatctcatc tccatcatct cattcctggc | |
| | 1981 | gctcgtgccc tgcctcagat ccatgggcgc aagtggacgg gcacgcacga gacgtggcta | |
| | 2041 | gtagtactcc agtatagcaa tgcttattag cctcactcac tcaccagtgc tatactagta | |
| | 2101 | gggcagccag cagcaacaca tgacacggga ggagacagca ccacccggcc ggggtccctc | |
| | 2161 | ccattgttag gttaggagcc tgttgctgtc acctgctgtt gcgagttaac aacatgtaac | |
| | 2221 | atgctaaaga ttcgttcttc cttcctttcc acaacacagg tcgctttctg acttgcttgc | |
| | 2281 | ttctcgttct cgacacaatg caatgcagca gctagcagcc catcacctag tcctcaccca | |
| | 2341 | tctttctgta gtactgcttc cattttcacc atggcttggc tcaccggccg gggtggtagt | |
| | 2401 | agaaagtaga attcccagga atctagtaag agaacaagtc ccaagttggc attttgtcag | |
| | 2461 | gaatgtcaag ttgcgacttg cgagatccca tcccaagaat tttctgcaat gggagaggag | |
| | 2521 | aggaccatgg ccgttgtgtt tattttactg tcaagcgcag gcatagggcg gttcctcctt | |
| | 2581 | ccttgagtca ttaacacaaa tcctgcgtgg agtgtattaa caagagtttg tagcactacc | |
| | 2641 | aaaccacatg ggccactgac tctgcagctg aggagtagta aaatagtgca gtgtggaggc | |
| | 2701 | cggcccatcc tcctatgcac atgacatgat ccgacggaga tagtagaata gatccgacat | |
| | 2761 | gatatttttt ttaattcatg acaatcatga taaaaaaaaa gcaaagtcaa acaaggtcaa | |
| | 2821 | ttatgataaa aaaaatgcaa tgtgcaggtc ggtgagcgcg gtctgcagct gtctggcggt | |
| | 2881 | cagaagcagc gcatcgccat tgcccgcgcc atgctcaaga accccggcat cttgctgctg | |
| | 2941 | gacgaggcga cgagcgcgct ggactctgag tcggagaagc tggtgcagga ggcgctggac | |
| | 3001 | cggttcatga tcgggcgcac gaccctggtg atcgcgcacc ggctgtcgac gatccgcaag | |
| | 3061 | gccgacctgg tggcggtgct gcacggcggc gccgtgtcgg agattgggac gcacgaggag | |
| | 3121 | ctgatgggca agggcgagga cggcgcgtac gcgcggctga tccggatgca ggagcaggcc | |
| | 3181 | gcgcaggagg tggccgcccg tcggagcagc gcgcgcaact cggtgagcgc gcgcaactcg | |
| | 3241 | gtgagctcgc ccatcatgac gcgcaactcc tcctacgggc ggtcgcccta ctcgcgccgc | |
| | 3301 | ctctccgact tctccaacgc cgacttccac taccacggcg gtggtgaact accggagggt | |
| | 3361 | aataccaaga agatgattca tcagagggtt gcgttccggg cgggggcgag ctctttcctg | |
| | 3421 | cgtctggcga agatgaactc gcccgagtgg gggtacgctc tggtgggctc gctgggctcc | |
| | 3481 | atggtgtgcg gctccttcag cgccatcttc gcctacgttc tgagcgccgt gctgagcgtg | |
| | 3541 | tactacgccc ccgacccggg ccacatgcgg cgggagatcg ccaagtactg ctacctgctg | |
| | 3601 | atgggcatgt cttccgcggc gctggtgtgc aacacggtgc agcacgtgtt ctgggacacg | |
| | 3661 | gtgggcgaga acctgacgaa gcgcgtccgg gagcgcatgc tcggcgccgt gctccgcaac | |
| | 3721 | gagatggcgt ggttcgacgc cgaggagaac gccagcgccc gggtggccgc gaggctggcg | |
| | 3781 | ctggacgcgc agaacgtgcg ctccgccatc ggcgaccgca tctcggtgat cgtgcagaac | |
| | 3841 | tcggcgctgc tgctggtggc gtgcacggcc gggttcgtgc tgcagtggcg cctcgccctg | |
| | 3901 | gtgctgctgg gcgtgttccc gctggtggtg gccgccaccg tgctgcagaa gatgttcatg | |
| | 3961 | aagggcttct cgggggacct ggaggcggcc cacgcccgcg ccacgcagat cgccggcgag | |
| | 4021 | gccgtcgcca acctgcgcac cgtcgccgcg ttcaacgccg aggccaagat cgcgggactc | |
| | 4081 | ttcgcgggca acctgcgggg cccgctgcgg cggtgcctct ggaagggcca ggtggccggg | |
| | 4141 | tgcggctacg gggtggcgca gttcctgctg tacgcgtcct acgcgctggg gctctggtac | |
| | 4201 | gcggcgtggc tggtgaagca cggcgtctcc gacttctccc gcaccatccg cgtcttcatg | |
| | 4261 | gtgctcatgg tctccgccaa cggcgccgcc gagacgctca cgctggcgcc ggacttcgtc | |
| | 4321 | aggggcggac gcgccatgcg ctccgtcttc gagaccatcg acaggcgcac cgaggggac | |
| | 4381 | cccgacgacc cggacgcggc gccattgcaa ctgccattat tgacaggggt ggagctgcgg | |
| | 4441 | cacgtggact tctgctaccc gtcgcggccg gaggtgcagg tgctgcagga cctcagcctg | |
| | 4501 | cgcgcgcgcg cggggaagac gctggcgctc gtggggccca gcggctgcgg caagagctcc | |
| | 4561 | gtgctggcgc tcatccagcg cttctacgag cccacctcgg gccgcgtcct cctcgacggc | |
| | 4621 | cgggacgcgc gcaagtacaa cctccgggcg ctgcgccgcg ccgtcgccgt ggtgccgcag | |
| | 4681 | gagcccttcc tgttcgcggc gagcatccac gacaacatcg cctacggccg cgaggggggc | |
| | 4741 | gccacggagg cggaggtgct ggaggcggcc acgcaggcca acgcgcacaa gttcatctcc | |
| | 4801 | gcgctgcccg acggctacag gacgcaggtc ggggagcgcg gggtgcagct ctccggcggc | |
| | 4861 | cagcgccagc gcatcgccgt ggcgcgcgcc ctcgtcaagc aggccgccgt tctgctgctg | |
| | 4921 | gacgaggcca ccagcgcgct ggacgccgag tcggagcgca gcgtgcagca ggcgctggac | |
| | 4981 | cgccacgcca agacccgcag caccaccacc atcgtcgtcg cgcaccgcct cgccaccgtg | |
| | 5041 | cgcaacgccc acaccatcgc cgtcatcgac gagggcaagg tcgtcgagca gggatcgcac | |
| | 5101 | tcgcacctgc tcaaccacca cccggacgga acctacgcgc gcatgctcca actacagcga | |
| | 5161 | ctcacctcat ccacttccta a | |
| 31 | MSASSDPEEIRGRVVVLGADADELARPELEAFHLPSPTLEEAGASVGGPAA | | ATP-binding |
| | ANPPVVAPLETELPPNAMPSSSSSSNSNSNEQNKEEKKKGVALAPAPLGSL | | cassette |
| | FRFADGLDCVLMSVGTLGALVHGCSLPVELRFFAELVDSFGSHADDPDTMV | | type B1 |
| | RLVARYALYFLVVGAAIWASSWAEISCWMWTGERQSTRMRIRYLESALRQD | | (ABCB1) |
| | VSFFDTDVRTSDVIYAINADAVIVQDAISEKLGNLIHYMATFVAGFVVGFT | | auxin |
| | AAWQLALVTLAVVPLIAVIGGLSAAALAKLSSRSQDALAEASNIAEQAVAQ | | transporter |
| | IRTVQAFVGEERAMRAYSLALGMAQRIGYRSGFAKGLGLGGTYFTVFCCYA | | protein |
| | LLLWYGGHLVRRHHTNGGLAIATMESVMIGGLALGQSAPSMAAFAKARVAA | | (DW3B) |
| | AKIFRIIDHLAVVHGDHVQLPSVTGRVEMRGVDEAYPSRPDIPVLRGFSLT | | protein) |
| | VPPGKTIALVGSSGSGKSTVVSLIERFYDPSAGEILLDGHDLKSLNLRWLR | | encoded by |
| | QQIGLVSQEPTLFATSIKENLLLGRDSQSATLAEMEEAARVANAHSFIIKL | | Eragrostis |
| | PDGYDTQVGERGLQLSGGQKQRIAIARAMLKNPGILLLDEATSALDSESEK | | tef Dw3B |
| | LVQEALDREMIGRTTLVIAHRLSTIRKADLVAVLHGGAVSEIGTHDELMAK | | Gene (B |
| | GEDGAYARLIRMQQEQAAAQEVAARRSSARPSSARNSVSSPIMTRNSSYGR | | genome) |
| | SPYSRRLSDESNADSHHYYHGGELIESNNKAHHQRRIAFRAGASSFWRLAK | | |
| | MNSPEWGYALVGSLGSMVCGSFSAIFAYALSAVLSVYYAPDPGYMRRQIGK | | |
| | YCYLLMGMSSAALVENTVQHAFWDTVGENLTKRVRERMFGAVLRNEVAWFD | | |
| | AEENASARVAARLALDAQNVRSAIGDRISVIVQNSALLLVACTAGFVLQWR | | |
| | LALVLLAVEPLVVAATVLQKMEMKGESGDLEAAHARATQIAGEAVANLRTV | | |
| | AAFNAQAKIAGLFAANLRGPLRRCFWKGQAAGCGYGLAQFLLYASYALGLW | | |
| | YAAWLVKHGVSDESRAIRVFMVLMVSANGAAETLTLAPDFVKGGRAMRSVF | | |
| | ETIDRRTETEPDDPDAAPLPSDAVSVELRHVDFCYPSRPDVRVLQDLSLRA | | |
| | RAGKTLALVGPSGCGKSSVLALIQRFYEPTSGRVLLDGRDARKYNLRALRR | | |
| | AIAVVPQEPFLFAATIHDNIAYGREGATEAEVVEAATQANAHKFISALPDG | | |
| | YRTQVGERGVQLSGGQRQRIAVARALVKQAAVLLLDEATSALDAESERSVQ | | |
| | QALDRHAKTRSTTTIVVAHRLATVRDAHTIAVIDDGKVVEQGSHSHLLNHH | | |
| | PDGTYARMLHLQRLTAPSTS | | |
| 32 | 1 | atgtcagcta gtagcgaccc ggaggagatc cggggccgcg tggtggtcct cggcgccgat | Eragrostis |
| | 61 | gccgacgagt tggctcgccc cgagctggag gccttccacc tcccatctcc gacattagag | tef Dw3B |
| | 121 | gaggcaggag cctctgtcgg aggccctgct gctgctaatc ctccggtggt ggcgccattg | Gene (B |
| | 181 | gagacggagc tgcctcccaa tgcgatgcct tcttcttcct cctcctccaa cagcaacagc | genome) |
| | 241 | aatgagcaga acaaggagga gaagaagaag ggggtggcgt tggcgcctgc tcctcttggg | Encoding |
| | 301 | tcgctgttcc gtttcgccga cggtcttgac tgcgttctga tgtcggtggg cacgttgggc | ABCB1 |
| | 361 | gcgcttgtcc acggctgctc cctccccgtg ttcctccgct tcttcgcgga gctggtggac | protein |
| | 421 | tcgttcggct cccacgccga cgacccggac accatggtgc ggctggtggc caggtacgcg | (DW3B) |
| | 481 | ttgtattttc tggtggtggg cgcggccatc tgggcgtcct cctgggcgga gatctcgtgc | protein) |
| | 541 | tggatgtgga cgggggagcg gcagtcgacg cggatgcgga tccggtacct ggagtcggcg | |
| | 601 | ctccgtcagg acgtgtcctt cttcgacacc gacgtccgca cctccgacgt gatctacgcc | |
| | 661 | atcaacgccg acgccgtcat cgtgcaggac gccatcagcg agaagctcgg caacctcatc | |
| | 721 | cactacatgg ccaccttcgt ggccggattc gtggtggggt tcacggccgc ctggcagctg | |
| | 781 | gcgctggtga cgctggccgt ggtgccgctc atcgccgtga tcggcggcct cagcgcggcg | |
| | 841 | gcgctggcga agctgtcgtc gcggagccag gacgcgctgg cggaggcgag caacatcgcg | |
| | 901 | gagcaggcgg tggcgcagat acggacggtg caggcgttcg tcggggagga gcgcgcaatg | |
| | 961 | cgggcctact cgttggctct aggaatggcg cagaggatcg ggtaccgcag cggcttcgcc | |
| | 1021 | aagggcctgg gcctgggcgg cacctacttc accgtcttct gctgctacgc gctgctgctc | |
| | 1081 | tggtacggcg gccacctcgt gcgccgccac cacaccaacg gcggcctcgc catcgccacc | |
| | 1141 | atgttctccg tcatgatcgg gggcctcgcg ctgggccagt ccgcgcccag catggccgcc | |
| | 1201 | ttcgcaaagg cgcgcgtcgc cgcggccaag atcttcagga tcatcgacca ccttgccgtc | |
| | 1261 | gtccatggcg accatgtcca gctcccttcc gtgacggggc gggtggagat gcggggcgtt | |
| | 1321 | gacttcgcct acccgtcgcg accggacatc cccgtcctgc gcggcttctc cctcaccgtg | |
| | 1381 | ccgccaggca agaccatcgc gctcgtcggg agctcaggct ccggaaagag cacagtcgtc | |
| | 1441 | tccctcattg agaggttcta cgacccaagt gcaggtacac aaaaaaactt ccttggttga | |
| | 1501 | aaatttctct tgttcgttcg ttcacacaca ttcatcgatt caaaaacata tgcttcttac | |
| | 1561 | taattactac tactaggtga gattctgctt gacgggcacg acctcaagtc cctgaacctg | |
| | 1621 | cgctggctcc ggcagcagat cgggctggtg agccaggagc cgacgctgtt cgccacgagc | |
| | 1681 | atcaaggaga acctgctgct gggccgcgac agccagagcg ccaccctcgc cgagatggag | |
| | 1741 | gaggccgcca gggtcgccaa cgcccactca ttcatcatca agctgcccga cggctacgac | |
| | 1801 | acccaggtat gtacagtatg cttcgctttg ctccatctca ctcaatgcca ccctcggaaa | |
| | 1861 | ggaagaaaag cttttcctcc atcatctcat tccttcctgg cgctctcaga tccacgggca | |
| | 1921 | cgcacgagac tgggcgggct actagagtac taccagtata ctagcaatgc ttgacctcac | |
| | 1981 | tcaccagtac tagtactcta gtagggcagc aacacatgac acgggagaca gcaccacccg | |
| | 2041 | gccggggtcc ctcccattgt taggttagga ggctgttgct gtcacctgtt gttgcgagtt | |
| | 2101 | actcctaaca acatgtaaca tgctaaagat tctttcttcc tttctttcca caacacaggt | |
| | 2161 | cgctttctga gttgcttgct tctcgacaca atgcaatgca gcagcagcag cagctagcag | |
| | 2221 | cccatcactc atctttttgt gctgcattca ttttcaccat ggcttgcctc aaaaaagaag | |
| | 2281 | ctactcactc cagtaaatca tccctctcgt ggtggtagta ctccaaagta gaattcccag | |
| | 2341 | gaatctagta acggaacaag tcccaagttg gcattttgtc aggaatgtca agttgcgaga | |
| | 2401 | tcccatccca agaattttgt ggagagagag agagagagag agggagcaat gggagaggag | |
| | 2461 | gaggaccatg gccgttctgt tgttatttta gtgtcaagtg caggcagagt gcagttaaca | |
| | 2521 | aatcctgagt agtttattaa caagagtttg tactagtacc aaaccacatg gggcgccact | |
| | 2581 | gactctgcat tgctagttaa aaaatattgc agtgtggtgg aggccgggcc ggcccagccc | |
| | 2641 | atcctcctgt gcacatggca tgatccgacg gagatagatc ctgcattagt ttagtttaaa | |
| | 2701 | cctttctttc tgttctttct tctagtagta gtaggagtat cccagtcaaa ctaagcactc | |
| | 2761 | tgctggtcaa aaaaaaaaaa aaacaggcgg agtactcctg atttaattaa ctgatgatga | |
| | 2821 | tgatgacatt tgccttaagt tagtaattca tgatgagaat aatagaagga aaaacaaagt | |
| | 2881 | caatggatgt gcatgcaggt cggtgagcgc ggtctgcagc tgtctggcgg tcagaagcag | |
| | 2941 | cgcatcgcca tcgcgcgcgc catgctcaag aaccctggga tcttgctgct ggacgaggcg | |
| | 3001 | acgagcgcgc tggactcgga gtccgagaag ctggtgcagg aggcgctgga ccggttcatg | |
| | 3061 | atcgggcgca cgaccctggt gatcgcgcac cggctgtcga cgatccgcaa ggccgacctg | |
| | 3121 | gtggccgtgc tgcacggcgg cgccgtgtcg gagattggga cgcacgacga gctgatggcc | |
| | 3181 | aagggcgagg acggcgcgta cgcgcggctg atccggatgc agcaggagca ggccgccgcg | |
| | 3241 | caggaggtgg ccgcccgtcg gagcagcgcg cggccgtcga gcgcgcgcaa ctcggtcagc | |
| | 3301 | tcgcccatca tgacgcgcaa ctcctcctac gggcggtcgc cctactcgcg gcgcctctcc | |
| | 3361 | gacttctcca acgccgactc ccaccactac taccatggtg gtgaacttat tgagagtaac | |
| | 3421 | aacaaggctc atcatcaaag gaggaggatt gcgttccggg ctggggcgag ctcgttctgg | |
| | 3481 | aggctggcca agatgaactc gcccgagtgg gggtacgcgc tggtggggtc cctgggctcc | |
| | 3541 | atggtgtgcg gctccttcag cgccatcttc gcctacgcgc tgagcgccgt gctgagcgtg | |
| | 3601 | tactacgccc ccgacccggg ttacatgcgg cggcagatcg gcaagtactg ctacctgctg | |
| | 3661 | atgggcatgt cttcggcggc gctggtgttc aacacggtgc agcacgcgtt ctgggacacg | |
| | 3721 | gtgggcgaga acctgacgaa gcgcgtccgg gagcgcatgt tcggcgccgt gctccgcaac | |
| | 3781 | gaggtggcgt ggttcgacgc cgaggagaac gccagcgccc gggtggccgc caggctggcg | |
| | 3841 | ctggacgcgc agaacgtgcg ttccgccatc ggcgaccgca tctcggtgat cgtgcagaac | |
| | 3901 | tcggcgctgc tgctggtggc ctgcacggcg gggttcgtgc tgcagtggcg cctggcgctg | |
| | 3961 | gtgctcctgg ccgtgttccc gctggtggtg gccgccaccg tgctgcagaa gatgttcatg | |
| | 4021 | aagggcttct cgggcgacct ggaggcggcc cacgcccgcg ccacgcagat cgccggcgag | |
| | 4081 | gccgtcgcca acctgcgcac cgtcgccgcc ttcaacgccc aggccaagat cgcgggcctc | |
| | 4141 | ttcgccgcca acctgcgggg cccgctgcgg cgctgcttct ggaagggcca ggccgccggc | |
| | 4201 | tgcggctacg ggctggccca gttcctgctc tacgcgtcct acgcgctggg cctctggtac | |
| | 4261 | gcggcctggc tcgtcaagca cggcgtctcc gacttctccc gcgccatccg ggtcttcatg | |
| | 4321 | gtgctcatgg tctccgccaa cggcgccgcc gagacgctca ccctggcgcc ggacttcgtc | |
| | 4381 | aaaggcggcc gcgccatgcg atccgtcttc gagaccatcg acaggcgcac cgagacggag | |
| | 4441 | cccgacgacc cggacgccgc gccactgcca tcggacgcgg tctccgtgga gctgcggcac | |
| | 4501 | gtggacttct gctacccgtc gcggccggac gtgcgggtgc tgcaggacct cagcctgcgc | |
| | 4561 | gcgcgcgcgg ggaagacgct ggcgctcgtg gggcccagcg ggtgcggcaa gagctccgtg | |
| | 4621 | ctcgcgctca tccagcgctt ctacgagccc acctcgggcc gcgtcctcct cgacggccgc | |
| | 4681 | gacgcgcgca agtacaacct ccgggcgctg cggcgcgcca tcgccgtcgt gccgcaggag | |
| | 4741 | cccttcctct tcgcggccac catccacgac aacatcgcct acggccgcga gggcgccacg | |
| | 4801 | gaggcggagg tggtggaggc ggccacgcag gccaacgcgc acaagttcat ctccgcgctg | |
| | 4861 | cccgacgggt acaggacgca ggtcggcgag cgcggggtgc agctctccgg cgggcagcga | |
| | 4921 | cagcgcatcg ccgtggctcg ggcgcttgtc aaacaggccg ccgttctgct cctcgacgag | |
| | 4981 | gccaccagcg cgctggacgc cgagtcggag cgcagcgtgc agcaggcgct ggaccgccac | |
| | 5041 | gccaagaccc gtagcaccac caccatcgtc gtcgcgcacc gcctcgccac cgtccgcgac | |
| | 5101 | gcacacacca tcgccgtcat cgacgacggc aaggtcgtcg agcagggatc gcactcacac | |
| | 5161 | ctgctcaacc accaccccga cggaacctac gcgcgcatgc tccacctcca gcgactcacc | |
| | 5221 | gcgccatcca cttcctaa | |
| 33 | 1 | atgtctagca gcgacccgga ggagatccgg gggcgcgtgg tcgtcctcgg cgccgatgcg | Eragrostis |
| | 61 | gacgagttgg ctcgccccga gctggaggcc ttccacctcc cgtctccgac actagctcag | tef Dw3A |
| | 121 | gaggcaggat ctgtcggagg cccaggccct gctgctgctg ctcccgtggt ggcgccattg | Gene (A |
| | 181 | gagacggagc tgcctcccaa tgccaccatg ccttcttctt cctccgcctc ctcgaacagc | Genome) |
| | 241 | aacagcaatg agcagaacaa ggagcaggag aatacgaaga agaagggggt gtctttggcg | comprising |
| | 301 | cctgctcctc ttggttcgct gttccgtttc gccgacggtc tggactgcgt tctgatgtcg | nucleic acid |
| | 361 | gtgggcacgt tgggcgcgct tgtccacggt tgctccctcc cggtgttcct ccgcttcttc | modification |
| | 421 | gcggacctcg tcgactcgtt cggctcccac gccgacgacc cggacaccat ggtgcggctg | that results |
| | 481 | gtggccaagt acgcgctcta cttcctggtg gtgggcgcgg ccatctgggc gtcctcctgg | in a |
| | 541 | gcggagatct cgtgctggat gtggaccggc gagcggcagt cgacgcggat gcggatccgg | reduction in |
| | 601 | tacctggagt cggcgctccg gcaggacgtg tccttcttcg acaccgacgt ccgcacctcc | auxin |
| | 661 | gacgtgatct acgccatcaa cgcggacgcc gtgatcgtgc aggacgccat cagcgagaag | transport |
| | 721 | ctcggcaacc tcatccacta catggccacc ttcgtggccg ggttcgtggt cgggttcacg | activity |
| | 781 | gcggcatggc agctggcgct ggtgacgctg gccgtggtgc cgctcatcgc cgtgatcggc | |
| | 841 | ggcctcagcg cggcggcgct ggcgaagctg tcgtcgcgga gccaggacgc gctggcggag | |
| | 901 | gcgagcaaca tcgcggagca ggcggtggcg cagatacgga cggtgcaggc cttcgtcggg | |
| | 961 | gaggagcgcg caatgcgggc ctactcgttg gcgctggccg cggcgcagag gatcggatac | |
| | 1021 | cgcagcggct tcgccaaggg cctgggcctg ggcggcacct acttcaccgt cttctgctgc | |
| | 1081 | tacgcgctgc tgctctggta cggcggcctc ctcgtgcgac gccaccacac caacggcggc | |
| | 1141 | ctcgccatcg ccaccatgtt ctccgtcatg atcgggggcc tcgcgctggg ccagtcggcg | |
| | 1201 | cccagcatgg ccgccttcgc aaaggcgcgc gtcgccgcgg ccaagatctt caaaatcatc | |
| | 1261 | gaccacaagc ccctctccgt cgtcgtccat ggcgacgacg atgtccaact cccttccgtg | |
| | 1321 | acggggcggg tggagatgcg gggcgttgac ttcgcctacc cgtcgcggcc ggacgtcccc | |
| | 1381 | gtcctgcgcg gcttctccct caccgtgccg cccggcaaga ccatcgcgct cgtcgggagc | |
| | 1441 | tcaggctccg ggaagagcac ggtcgtctcc ctcatcgaga ggttctacga cccaagtgca | |
| | 1501 | ggtacacaac tacttaacgt cttagaagtg tctgtcagtc aattgtcttc ttcattcaca | |
| | 1561 | atcgtattca ttgattgaaa gaaaaatatg cttctcaggc gagattctgc ttgacgggca | |
| | 1621 | cgacctcaag tccctcaacc tgcggtggct ccggcagcag atcgggctgg tgagccagga | |
| | 1681 | gccgacgctg ttcgccacca gcatcaagga gaacctgctg ctgggccggg acagccacag | |
| | 1741 | cgccaccctc gccgagatgg aggaggccgc aagggtcgcc aacgcccact cattcatcat | |
| | 1801 | caagctgccc gacggatacg acacccaggt atgtactagc accgttgctt tcctttcaca | |
| | 1861 | ttcctctcct ctcctgagca actagatatg tactagcacc attgctccat ctcaatgcca | |
| | 1921 | ccatcctcaa aaaagaagga aagcttttct ccatctcatc tccatcatct cattcctggc | |
| | 1981 | gctcgtgccc tgcctcagat ccatgggcgc aagtggacgg gcacgcacga gacgtggcta | |
| | 2041 | gtagtactcc agtatagcaa tgcttattag cctcactcac tcaccagtgc tatactagta | |
| | 2101 | gggcagccag cagcaacaca tgacacggga ggagacagca ccacccggcc ggggtccctc | |
| | 2161 | ccattgttag gttaggagcc tgttgctgtc acctgctgtt gcgagttaac aacatgtaac | |
| | 2221 | atgctaaaga ttcgttcttc cttcctttcc acaacacagg tcgctttctg acttgcttgc | |
| | 2281 | ttctcgttct cgacacaatg caatgcagca gctagcagcc catcacctag tcctcaccca | |
| | 2341 | tctttctgta gtactgcttc cattttcacc atggcttggc tcaccggccg gggtggtagt | |
| | 2401 | agaaagtaga attcccagga atctagtaag agaacaagtc ccaagttggc attttgtcag | |
| | 2461 | gaatgtcaag ttgcgacttg cgagatccca tcccaagaat tttctgcaat gggagaggag | |
| | 2521 | aggaccatgg ccgttgtgtt tattttactg tcaagcgcag gcatagggcg gttcctcctt | |
| | 2581 | ccttgagtca ttaacacaaa tcctgcgtgg agtgtattaa caagagtttg tagcactacc | |
| | 2641 | aaaccacatg ggccactgac tctgcagctg aggagtagta aaatagtgca gtgtggaggc | |
| | 2701 | cggcccatcc tcctatgcac atgacatgat ccgacggaga tagtagaata gatccgacat | |
| | 2761 | gatatttttt ttaattcatg acaatcatga taaaaaaaaa gcaaagtcaa acaaggtcaa | |
| | 2821 | ttatgataaa aaaaatgcaa tgtgcaggtc ggtgagcgcg gtctgcagct gtctggcggt | |
| | 2881 | cagaagcagc gcatcgccat tgcccgcgcc atgctcaaga accccggcat cttgctgctg | |
| | 2941 | gacgaggcga cgagcgcgct ggactctgag tcggagaagc tggtgcagga ggcgctggac | |
| | 3001 | cggtttcatga tcgggcgcac gaccctggtg atcgcgcacc ggctgtcgac gatccgcaag | |
| | 3061 | gccgacctgg tggcggtgct gcacggcggc gccgtgtcgg agattgggac gcacgaggag | |
| | 3121 | ctgatgggca agggcgagga cggcgcgtac gcgcggctga tccggatgca ggagcaggcc | |
| | 3181 | gcgcaggagg tggccgcccg tcggagcagc gcgcgcaact cggtgagcgc gcgcaactcg | |
| | 3241 | gtgagctcgc ccatcatgac gcgcaactcc tcctacgggc ggtcgcccta ctcgcgccgc | |
| | 3301 | ctctccgact tctccaacgc cgacttccac taccacggcg gtggtgaact accggagggt | |
| | 3361 | aataccaaga agatgattca tcagagggtt gcgttccggg cgggggcgag ctctttcctg | |
| | 3421 | cgtctggcga agatgaactc gcccgagtgg gggtacgctc tggtgggctc gctgggctcc | |
| | 3481 | atggtgtgcg gctccttcag cgccatcttc gcctacgttc tgagcgccgt gctgagcgtg | |
| | 3541 | tactacgccc ccgacccggg ccacatgcgg cgggagatcg ccaagtactg ctacctgctg | |
| | 3601 | atgggcatgt cttccgcggc gctggtgtgc aacacggtgc agcacgtgtt ctgggacacg | |
| | 3661 | gtgggcgaga acctgacgaa gcgcgtccgg gagcgcatgc tcggcgccgt gctccgcaac | |
| | 3721 | gagatggcgt ggttcgacgc cgaggagaac gccagcgccc gggtggccgc gaggctggcg | |
| | 3781 | ctggacgcgc agaacgtgcg ctccgccatc ggcgaccgca tctcggtgat cgtgcagaac | |
| | 3841 | tcggcgctgc tgctggtggc gtgcacggcc gggttcgtgc tgcagtggcg cctcgccctg | |
| | 3901 | gtgctgctgg gcgtgttccc gctggtggtg gccgccaccg tgctgcagaa gatgttcatg | |
| | 3961 | aagggcttct cgggggacct ggaggcggcc cacgcccgcg ccacgcagat cgccggcgag | |
| | 4021 | gccgtcgcca acctgcgcac cgtcgccgcg ttcaacgccg aggccaagat cgcgggactc | |
| | 4081 | ttcgcgggca acctgcgggg cccgctgcgg cggtgcctct ggaagggcca ggtggccggg | |
| | 4141 | tgcggctacg gggtggcgca gttcctgctg tacgcgtcct acgcgctggg gctctggtac | |
| | 4201 | gcggcgtggc tggtgaagca cggcgtctcc gacttctccc gcaccatccg cgtcttcatg | |
| | 4261 | gtgctcatgg tctccgccaa cggcgccgcc gagacgctca cgctggcgcc ggacttcgtc | |
| | 4321 | aggggcggac gcgccatgcg ctccgtcttc gagaccatcg acaggcgcac cgaggcggac | |
| | 4381 | cccgacgacc cggacgcggc gccattgcaa ctgccattat tgacaggggt ggagctgcgg | |
| | 4441 | cacgtggact tctgctaccc gtcgcggccg gaggtgcagg tgctgcagga cctcagcctg | |
| | 4501 | cgcgcgcgcg cggggaagac gctggcgctc gtggggccca gcggctgcgg caagagctcc | |
| | 4561 | gtgctggcgc tcatccagcg cttctacgag cccacctcgg gccgcgtcct cctcgacggc | |
| | 4621 | cgggacgcgc gcaagtacaa cctccgggcg ctgcgccgcg ccgtcgccgt ggtgccgcag | |
| | 4681 | gagcccttcc tgttcgcggc gagcatccac gacaacatcg cctacggccg cgaggggggc | |
| | 4741 | gccacggagg cggaggtgct ggaggcggcc acgcaggcca acgcgcacaa gttcatctcc | |
| | 4801 | gcgctgcccg acggctacag gacgcaggtc ggggagcgcg gggtgcagct ctccggcggc | |
| | 4861 | cagcgccagc gcatcgccgt ggcgcgcgcc ctcgtcaagc aggccgccgt tctgctgctg | |
| | 4921 | gacgaggcca ccagcgcgct ggacgccgag tcggagcgca gcgtgcagca ggcgctggac | |
| | 4981 | cgccacgcca agacccgcag caccaccacc atcgtcgtcg cgcaccgcct cgccaccgtg | |
| | 5041 | cgcaacgccc acaccatcgc cgtcatcgac gagggcaagg tcgtcgagca gggatcgcac | |
| | 5101 | tcgcacctgc tcaaccacca cccggacgga acctacgcgc gcatgctcca actacagcga | |
| | 5161 | ctcacctcat ccacttccta a | |
| 34 | 1 | atgtcagcta gtagcgaccc ggaggagatc cggggccgcg tggtggtcct cggcgccgat | Eragrostis |
| | 61 | gccgacgagt tggctcgccc cgagctggag gccttccacc tcccatctcc gacattagag | tef Dw3B |
| | 121 | gaggcaggag cctctgtcgg aggccctgct gctgctaatc ctccggtggt ggcgccattg | Gene (B |
| | 181 | gagacggagc tgcctcccaa tgcgatgcct tcttcttcct cctcctccaa cagcaacagc | Genome) |
| | 241 | aatgagcaga acaaggagga gaagaagaag ggggtggcgt tggcgcctgc tcctcttggg | comprising |
| | 301 | tcgctgttcc gtttcgccga cggtcttgac tgcgttctga tgtcggtggg cacgttgggc | nucleic acid |
| | 361 | gcgcttgtcc acggctgctc cctccccgtg ttcctccgct tcttcgcgga gctggtggac | modification |
| | 421 | tcgttcggct cccacgccga cgacccggac accatggtgc ggctggtggc caggtacgcg | that results |
| | 481 | ttgtattttc tggtggtggg cgcggccatc tgggcgtcct cctgggcgga gatctcgtgc | in a |
| | 541 | tggatgtgga cgggggagcg gcagtcgacg cggatgcgga tccggtacct ggagtcggcg | reduction in |
| | 601 | ctccgtcagg acgtgtcctt cttcgacacc gacgtccgca cctccgacgt gatctacgcc | auxin |
| | 661 | atcaacgccg acgccgtcat cgtgcaggac gccatcagcg agaagctcgg caacctcatc | transport |
| | 721 | cactacatgg ccaccttcgt ggccggattc gtggtggggt tcacggccgc ctggcagctg | activity |
| | 781 | gcgctggtga cgctggccgt ggtgccgctc atcgccgtga tcggcggcct cagcgcggcg | |
| | 841 | gcgctggcga agctgtcgtc gcggagccag gacgcgctgg cggaggcgag caacatcgcg | |
| | 901 | gagcaggcgg tggcgcagat acggacggtg caggcgttcg tcggggagga gcgcgcaatg | |
| | 961 | cgggcctact cgttggctct aggaatggcg cagaggatcg ggtaccgcag cggcttcgcc | |
| | 1021 | aagggcctgg gcctgggcgg cacctacttc accgtcttct gctgctacgc gctgctgctc | |
| | 1081 | tggtacggcg gccacctcgt gcgccgccac cacaccaacg gcggcctcgc catcgccacc | |
| | 1141 | atgttctccg tcatgatcgg gggcctcgcg ctgggccagt ccgcgcccag catggccgcc | |
| | 1201 | ttcgcaaagg cgcgcgtcgc cgcggccaag atcttcagga tcatcgacca ccttgccgtc | |
| | 1261 | gtccatggcg accatgtcca gctcccttcc gtgacggggc gggtggagat gggggcgtt | |
| | 1321 | gacttcgcct acccgtcgcg accggacatc cccgtcctgc gcggcttctc cctcaccgtg | |
| | 1381 | ccgccaggca agaccatcgc gctcgtcggg agctcaggct ccggaaagag cacagtcgtc | |
| | 1441 | tccctcattg agaggttcta cgacccaagt gcaggtacac aaaaaaactt ccttggttga | |
| | 1501 | aaatttctct tgttcgttcg ttcacacaca ttcatcgatt caaaaacata tgcttcttac | |
| | 1561 | taattactac tactaggtga gattctgctt gacgggcacg acctcaagtc cctgaacctg | |
| | 1621 | cgctggctcc ggcagcagat cgggctggtg agccaggagc cgacgctgtt cgccacgagc | |
| | 1681 | atcaaggaga acctgctgct gggccgcgac agccagagcg ccaccctcgc cgagatggag | |
| | 1741 | gaggccgcca gggtcgccaa cgcccactca ttcatcatca agctgcccga cggctacgac | |
| | 1801 | acccaggtat gtacagtatg cttcgctttg ctccatctca ctcaatgcca ccctcggaaa | |
| | 1861 | ggaagaaaag cttttcctcc atcatctcat tccttcctgg cgctctcaga tccacgggca | |
| | 1921 | cgcacgagac tgggcgggct actagagtac taccagtata ctagcaatgc ttgacctcac | |
| | 1981 | tcaccagtac tagtactcta gtagggcagc aacacatgac acgggagaca gcaccacccg | |
| | 2041 | gccggggtcc ctcccattgt taggttagga ggctgttgct gtcacctgtt gttgcgagtt | |
| | 2101 | actcctaaca acatgtaaca tgctaaagat tctttcttcc tttctttcca caacacaggt | |
| | 2161 | cgctttctga gttgcttgct tctcgacaca atgcaatgca gcagcagcag cagctagcag | |
| | 2221 | cccatcactc atctttttgt gctgcattca ttttcaccat ggcttgcctc aaaaaagaag | |
| | 2281 | ctactcactc cagtaaatca tccctctcgt ggtggtagta ctccaaagta gaattcccag | |
| | 2341 | gaatctagta acggaacaag tcccaagttg gcattttgtc aggaatgtca agttgcgaga | |
| | 2401 | tcccatccca agaattttgt ggagagagag agagagagag agggagcaat gggagaggag | |
| | 2461 | gaggaccatg gccgttctgt tgttatttta gtgtcaagtg caggcagagt gcagttaaca | |
| | 2521 | aatcctgagt agtttattaa caagagtttg tactagtacc aaaccacatg gggcgccact | |
| | 2581 | gactctgcat tgctagttaa aaaatattgc agtgtggtgg aggccgggcc ggcccagccc | |
| | 2641 | atcctcctgt gcacatggca tgatccgacg gagatagatc ctgcattagt ttagtttaaa | |
| | 2701 | cctttctttc tgttctttct tctagtagta gtaggagtat cccagtcaaa ctaagcactc | |
| | 2761 | tgctggtcaa aaaaaaaaaa aaacaggcgg agtactcctg atttaattaa ctgatgatga | |
| | 2821 | tgatgacatt tgccttaagt tagtaattca tgatgagaat aatagaagga aaaacaaagt | |
| | 2881 | caatggatgt gcatgcaggt cggtgagcgc ggtctgcagc tgtctggcgg tcagaagcag | |
| | 2941 | cgcatcgcca tcgcgcgcgc catgctcaag aaccctggga tcttgctgct ggacgaggcg | |
| | 3001 | acgagcgcgc tggactcgga gtccgagaag ctggtgcagg aggcgctgga ccggttacatg | |
| | 3061 | atcgggcgca cgaccctggt gatcgcgcac cggctgtcga cgatccgcaa ggccgacctg | |
| | 3121 | gtggccgtgc tgcacggcgg cgccgtgtcg gagattggga cgcacgacga gctgatggcc | |
| | 3181 | aagggcgagg acggcgcgta cgcgcggctg atccggatgc agcaggagca ggccgccgcg | |
| | 3241 | caggaggtgg ccgcccgtcg gagcagcgcg cggccgtcga gcgcgcgcaa ctcggtcagc | |
| | 3301 | tcgcccatca tgacgcgcaa ctcctcctac gggcggtcgc cctactcgcg gcgcctctcc | |
| | 3361 | gacttctcca acgccgactc ccaccactac taccatggtg gtgaacttat tgagagtaac | |
| | 3421 | aacaaggctc atcatcaaag gaggaggatt gcgttccggg ctggggcgag ctcgttctgg | |
| | 3481 | aggctggcca agatgaactc gcccgagtgg gggtacgcgc tggtggggtc cctgggctcc | |
| | 3541 | atggtgtgcg gctccttcag cgccatcttc gcctacgcgc tgagcgccgt gctgagcgtg | |
| | 3601 | tactacgccc ccgacccggg ttacatgcgg cggcagatcg gcaagtactg ctacctgctg | |
| | 3661 | atgggcatgt cttcggcggc gctggtgttc aacacggtgc agcacgcgtt ctgggacacg | |
| | 3721 | gtgggcgaga acctgacgaa gcgcgtccgg gagcgcatgt tcggcgccgt gctccgcaac | |
| | 3781 | gaggtggcgt ggttcgacgc cgaggagaac gccagcgccc gggtggccgc caggctggcg | |
| | 3841 | ctggacgcgc agaacgtgcg ttccgccatc ggcgaccgca tctcggtgat cgtgcagaac | |
| | 3901 | tcggcgctgc tgctggtggc ctgcacggcg gggttcgtgc tgcagtggcg cctggcgctg | |
| | 3961 | gtgctcctgg ccgtgttccc gctggtggtg gccgccaccg tgctgcagaa gatgttcatg | |
| | 4021 | aagggcttct cgggcgacct ggaggcggcc cacgcccgcg ccacgcagat cgccggcgag | |
| | 4081 | gccgtcgcca acctgcgcac cgtcgccgcc ttcaacgccc aggccaagat cgcgggcctc | |
| | 4141 | ttcgccgcca acctgcgggg cccgctgcgg cgctgcttct ggaagggcca ggccgccggc | |
| | 4201 | tgcggctacg ggctggccca gttcctgctc tacgcgtcct acgcgctggg cctctggtac | |
| | 4261 | gcggcctggc tcgtcaagca cggcgtctcc gacttctccc gcgccatccg ggtcttcatg | |
| | 4321 | gtgctcatgg tctccgccaa cggcgccgcc gagacgctca ccctggcgcc ggacttcgtc | |
| | 4381 | aaaggcggcc gcgccatgcg atccgtcttc gagaccatcg acaggcgcac cgagacggag | |
| | 4441 | cccgacgacc cggacgccgc gccactgcca tcggacgcgg tctccgtgga gctgcggcac | |
| | 4501 | gtggacttct gctacccgtc gcggccggac gtgcgggtgc tgcaggacct cagcctgcgc | |
| | 4561 | gcgcgcgcgg ggaagacgct ggcgctcgtg gggcccagcg ggtgcggcaa gagctccgtg | |
| | 4621 | ctcgcgctca tccagcgctt ctacgagccc acctcgggcc gcgtcctcct cgacggccgc | |
| | 4681 | gacgcgcgca agtacaacct ccgggcgctg cggcgcgcca tcgccgtcgt gccgcaggag | |
| | 4741 | cccttcctct tcgcggccac catccacgac aacatcgcct acggccgcga gggcgccacg | |
| | 4801 | gaggcggagg tggtggaggc ggccacgcag gccaacgcgc acaagttcat ctccgcgctg | |
| | 4861 | cccgacgggt acaggacgca ggtcggcgag cgcggggtgc agctctccgg cgggcagcga | |
| | 4921 | cagcgcatcg ccgtggctcg ggcgcttgtc aaacaggccg ccgttctgct cctcgacgag | |
| | 4981 | gccaccagcg cgctggacgc cgagtcggag cgcagcgtgc agcaggcgct ggaccgccac | |
| | 5041 | gccaagaccc gtagcaccac caccatcgtc gtcgcgcacc gcctcgccac cgtccgcgac | |
| | 5101 | gcacacacca tcgccgtcat cgacgacggc aaggtcgtcg agcagggatc gcactcacac | |
| | 5161 | ctgctcaacc accaccccga cggaacctac gcgcgcatgc tccacctcca gcgactcacc | |
| | 5221 | gcgccatcca cttcctaa | |

| | | |
|---|---|---|
| 35 | GGCCCGGACTTCGAGCCAATGGG | Sd1 gRNA1 |
| 36 | GCGACTTCTTCGAGGACAGCCGG | Sd1 gRNA2 |
| 37 | CCTGGAATATGGTGTGTCACAGG | Dw1 gRNA |
| 38 | TCGTGCGCCCGATCATGAACCGG | Dw3 gRNA |
| 39 | TGCAGTGCAGCGTGACCCGGTCGTGCCCCTCTCTAGAGATAATGAGCATTG | Cas9 and |
| | CATGTCTAAGTTATAAAAAATTACCACATATTTTTTTTGTCACACTTGTTT | Eragrostis tef |
| | GAAGTGCAGTTTATCTATCTTTATACATATATTTAAACTTTACTCTACGAA | SD-1 gRNA |
| | TAATATAATCTATAGTACTACAATAATATCAGTGTTTTAGAGAATCATATA | expression |
| | AATGAACAGTTAGACATGGTCTAAAGGACAATTGAGTATTTTGACAACAGG | cassettes used |
| | ACTCTACAGTTTTATCTTTTTAGTGTGCATGTGTTCTCCTTTTTTTTTGCA | to generate |
| | AATAGCTTCACCTATATAATACTTCATCCATTTTATTAGTACATCCATTTA | semidwarf tef |
| | GGGTTTAGGGTTAATGGTTTTTATAGACTAATTTTTTTAGTACATCTATTT | line |
| | TATTCTATTTTAGCCTCTAAATTAAGAAAACTAAAACTCTATTTTAGTTTT | >1-982 (Maize |
| | TTTATTTAATAATTTAGATATAAAATAGAATAAAATAAAGTGACTAAAAAT | Ubiquitin-1 |
| | TAAACAAATACCCTTTAAGAAATTAAAAAAACTAAGGAAACATTTTTCTTG | promoter) |
| | TTTCGAGTAGATAATGCCAGCCTGTTAAACGCCGTCGACGAGTCTAACGGA | >983-1995 |
| | CACCAACCAGCGAACCAGCAGCGTCGCGTCGGGCCAAGCGAAGCAGACGGC | (Maize |
| | ACGGCATCTCTGTCGCTGCCTCTGGACCCCTCTCGAGAGTTCCGCTCCACC | Ubiquitin-1, |
| | GTTGGACTTGCTCCGCTGTCGGCATCCAGAAATTGCGTGGCGGAGCGGCAG | intron-1) |
| | ACGTGAGCCGGCACGGCAGGCGGCCTCCTCCTCCTCTCACGGCACCGGCAG | >1996-2010 |
| | CTACGGGGGATTCCTTTCCCACCGCTCCTTCGCTTTCCCTTCCTCGCCCGC | sequence) |
| | CGTAATAAATAGACACCCCCTCCACACCCTCTTTCCCCAACCTCGTGTTGT | (intervening |
| | TCGGAGCGCACACACACACAACCAGATCTCCCCCAAATCCACCCGTCGGCA | >2011-6165 |
| | CCTCCGCTTCAAGGTACGCCGCTCGTCCTCCCCCCCCCCCCTCTCTACCTT | (Cas9 coding |
| | CTCTAGATCGGCGTTCCGGTCCATGGTTAGGGCCCGGTAGTTCTACTTCTG | sequence ) |
| | TTCATGTTTGTGTTAGATCCGTGTTTGTGTTAGATCCGTGCTGCTAGCGTT | >6166-6181 |
| | CGTACACGGATGCGACCTGTACGTCAGACACGTTCTGATTGCTAACTTGCC | >6182-6889 |
| | AGTGTTTCTCTTTGGGGAATCCTGGGATGGCTCTAGCCGTTCCGCAGACGG | (Octapine |
| | GATCGATTTCATGATTTTTTTTGTTTCGTTGCATAGGGTTTGGTTTGCCCT | (intervening |
| | TTTCCTTTATTTCAATATATGCCGTGCACTTGTTTGTCGGGTCATCTTTTC | sequence) |
| | ATGCTTTTTTTTGTCTTGGTTGTGATGATGTGGTCTGGTTGGGCGGTCGTT | synthase polyA |
| | CTAGATCGGAGTAGAATTAATTCTGTTTCAAACTACCTGGTGGATTTATTA | signal) |
| | ATTTTGGATCTGTATGTGTGTGCCATACATATTCATAGTTACGAATTGAAG | >6890-6893 |
| | ATGATGGATGGAAATATCGATCTAGGATAGGTATACATGTTGATGCGGGTT | (intervening |
| | TTACTGATGCATATACAGAGATGCTTTTTGTTCGCTTGGTTGTGATGATGT | sequence) |
| | GGTGTGGTTGGGCGGTCGTTCATTCGTTCTAGATCGGAGTAGAATACTGTT | >6894-7251 |
| | TCAAACTACCTGGTGTATTTATTAATTTTGGAACTGTATGTGTGTGTCATA | (Triticum |
| | CATCTTCATAGTTACGAGTTTAAGATGGATGGAAATATCGATCTAGGATAG | aestivum U6 |
| | GTATACATGTTGATGTGGGTTTTACTGATGCATATACATGATGGCATATGC | small nuclear |
| | AGCATCTATTCATATGCTCTAACCTTGAGTACCTATCTATTATAATAAACA | RNA gene |
| | AGTATGTTTTATAATTATTTTGATCTTGATATACTTGGATGATGGCATATG | promoter) |
| | CAGCAGCTATATGTGGATTTTTTTAGCCCTGCCTTCATACGCTATTTATTT | >7252-7255 |
| | GCTTGGTACTGTTTCTTTTGTCGATGCTCACCCTGTTGTTTGGTGTTACTT | (intervening |
| | CCTGCAgggcgatctattcgaATGGACAAGAAGTACTCGATCGGCCTCGAC | sequence) |
| | ATCGGGACGAACTCAGTTGGCTGGGCCGTGATCACCGACGAGTACAAGGTG | >7256-7275 |
| | CCCTCTAAGAAGTTCAAGGTCCTGGGGAACACCGACCGCCATTCCATCAAG | (Eragrostis |
| | AAGAACCTCATCGGCGCTCTCCTGTTCGACAGCGGGGAGACCGCTGAGGCT | tef SD1-gRNA1) |
| | ACGAGGCTCAAGAGAACCGCTAGGCGCCGGTACACGAGAAGGAAGAACAGG | >7276-7358 |
| | ATCTGCTACCTCCAAGAGATTTTCTCCAACGAGATGGCCAAGGTTGACGAT | (gRNA |
| | TCATTCTTCCACCGCCTGGAGGAGTCTTTCCTCGTGGAGGAGGATAAGAAG | scaffold) |
| | CACGAGCGGCATCCCATCTTCGGCAACATCGTGGACGAGGTTGCCTACCAC | >7359-7362 |
| | GAGAAGTACCCTACGATCTACCATCTGCGGAAGAAGCTCGTGGACTCCACC | (intervening |
| | GATAAGGCGGACCTCAGACTGATCTACCTCGCTCTGGCCCACATGATCAAG | sequence) |
| | TTCCGCGGCCATTTCCTGATCGAGGGGGATCTCAACCCAGACAACAGCGAT | >7363-7757 |
| | GTTGACAAGCTGTTCATCCAACTCGTGCAGACCTACAACCAACTCTTCGAG | (Triticum |
| | GAGAACCCGATCAACGCCTCTGGCGTGGACGCGAAGGCTATCCTGTCCGCG | aestivum U3 |
| | AGGCTCTCGAAGTCCAGGAGGCTGGAGAACCTGATCGCTCAGCTCCCAGGC | small nuclear |
| | GAGAAGAAGAACGGCCTGTTCGGGAACCTCATCGCTCTCAGCCTGGGGCTC | RNA gene |
| | ACCCCGAACTTCAAGTCGAACTTCGATCTCGCTGAGGACGCCAAGCTGCAA | promoter) |
| | CTCTCCAAGGACACCTACGACGATGACCTCGATAACCTCCTGGCCCAGATC | >7758-7761 |
| | GGCGATCAATACGCGGACCTGTTCCTCGCTGCCAAGAACCTGTCGGACGCC | (intervening |
| | ATCCTCCTGTCAGATATCCTCCGCGTGAACACCGAGATCACGAAGGCTCCA | sequence) |
| | CTCTCTGCCTCCATGATCAAGCGCTACGACGAGCACCATCAGGATCTGACC | >7762-7781 |
| | CTCCTGAAGGCGCTGGTCCGCCAACAGCTCCCGGAGAAGTACAAGGAGATT | (Eragrostis |
| | TTCTTCGATCAGTCGAAGAACGGCTACGCTGGGTACATCGACGGCGGGGCC | tef SD1-gRNA2) |
| | TCACAAGAGGAGTTCTACAAGTTCATCAAGCCAATCCTGGAGAAGATGGAC | >7782-7864 |
| | GGCACGGAGGAGCTCCTGGTGAAGCTCAACAGGGAGGACCTCCTGCGGAAG | (gRNA |
| | CAGAGAACCTTCGATAACGGCAGCATCCCCCACCAAATCCATCTCGGGGAG | scaffold) |
| | CTGCACGCCATCCTGAGAAGGCAAGAGGACTTCTACCCTTTCCTCAAGGAT | |
| | AACCGGGAGAAGATCGAGAAGATCCTGACCTTCAGAATCCCATACTACGTC | |
| | GGCCCTCTCGCGCGGGGGAACTCAAGATTCGCTTGGATGACCCGCAAGTCT | |
| | GAGGAGACCATCACGCCGTGGAACTTCGAGGAGGTGGTGGACAAGGGCGCT | |
| | AGCGCTCAGTCGTTCATCGAGAGGATGACCAACTTCGACAAGAACCTGCCC | |
| | AACGAGAAGGTGCTCCCTAAGCACTCGCTCCTGTACGAGTACTTCACCGTC | |
| | TACAACGAGCTCACGAAGGTGAAGTACGTCACCGAGGGCATGCGCAAGCCA | |
| | GCGTTCCTGTCCGGGGAGCAGAAGAAGGCTATCGTGGACCTCCTGTTCAAG | |
| | ACCAACCGGAAGGTCACGGTTAAGCAACTCAAGGAGGACTACTTCAAGAAG | |
| | ATCGAGTGCTTCGATTCGGTCGAGATCAGCGGCGTTGAGGACCGCTTCAAC | |
| | GCCAGCCTCGGGACCTACCACGATCTCCTGAAGATCATCAAGGATAAGGAC | |
| | TTCCTGGACAACGAGGAGAACGAGGATATCCTGGAGGACATCGTGCTGACC | |
| | CTCACGCTGTTCGAGGACAGGGAGATGATCGAGGAGCGCCTGAAGACGTAC | |
| | GCCCATCTCTTCGATGACAAGGTCATGAAGCAACTCAAGCGCCGGAGATAC | |
| | ACCGGCTGGGGGAGGCTGTCCCGCAAGCTCATCAACGGCATCCGGGACAAG | |
| | CAGTCCGGGAAGACCATCCTCGACTTCCTCAAGAGCGATGGCTTCGCCAAC | |
| | AGGAACTTCATGCAACTGATCCACGATGACAGCCTCACCTTCAAGGAGGAT | |
| | ATCCAAAAGGCTCAAGTGAGCGGCCAGGGGGACTCGCTGCACGAGCATATC | |
| | GCGAACCTCGCTGGCTCCCCCGCGATCAAGAAGGGCATCCTCCAGACCGTG | |
| | AAGGTTGTGGACGAGCTCGTGAAGGTCATGGGCCGGCACAAGCCTGAGAAC | |
| | ATCGTCATCGAGATGGCCAGAGAGAACCAAACCACGCAGAAGGGGCAAAAG | |
| | AACTCTAGGGAGCGCATGAAGCGCATCGAGGAGGGCATCAAGGAGCTGGGG | |
| | TCCCAAATCCTCAAGGAGCACCCAGTGGAGAACACCCAACTGCAGAACGAG | |
| | AAGCTCTACCTGTACTACCTCCAGAACGGCAGGGATATGTACGTGGACCAA | |
| | GAGCTGGATATCAACCGCCTCAGCGATTACGACGTCGATCATATCGTTCCC | |
| | CAGTCTTTCCTGAAGGATGACTCCATCGACAACAAGGTCCTCACCAGGTCG | |
| | GACAAGAACCGCGGCAAGTCAGATAACGTTCCATCTGAGGAGGTCGTTAAG | |
| | AAGATGAAGAACTACTGGAGGCAGCTCCTGAACGCCAAGCTGATCACGCAA | |
| | AGGAAGTTCGACAACCTCACCAAGGCTGAGAGAGGCGGGCTCTCAGAGCTG | |
| | GACAAGGCCGGCTTCATCAAGCGGCAGCTGGTCGAGACCAGACAAATCACG | |
| | AAGCACGTTGCGCAAATCCTCGACTCTCGGATGAACACGAAGTACGATGAG | |
| | AACGACAAGCTGATCAGGGAGGTTAAGGTGATCACCCTGAAGTCTAAGCTC | |
| | GTCTCCGACTTCAGGAAGGATTTCCAGTTCTACAAGGTTCGCGAGATCAAC | |
| | AACTACCACCATGCCCATGACGCTTACCTCAACGCTGTGGTCGGCACCGCT | |
| | CTGATCAAGAAGTACCCAAAGCTGGAGTCCGAGTTCGTGTACGGGGACTAC | |
| | AAGGTTTACGATGTGCGCAAGATGATCGCCAAGTCGGAGCAAGAGATCGGC | |
| | AAGGCTACCGCCAAGTACTTCTTCTACTCAAACATCATGAACTTCTTCAAG | |
| | ACCGAGATCACGCTGGCCAACGGCGAGATCCGGAAGAGACCGCTCATCGAG | |
| | ACCAACGGCGAGACGGGGGAGATCGTGTGGGACAAGGGCAGGGATTTCGCG | |
| | ACCGTCCGCAAGGTTCTCTCCATGCCCCAGGTGAACATCGTCAAGAAGACC | |
| | GAGGTCCAAACGGGGGGTTCTCAAAGGAGTCTATCCTGCCTAAGCGGAAC | |
| | AGCGACAAGCTCATCGCCAGAAAGAAGGACTGGGACCCAAAGAAGTACGGC | |
| | GGGTTCGACAGCCCTACCGTGGCCTACTCGGTCCTGGTTGTGGCGAAGGTT | |
| | GAGAAGGGCAAGTCCAAGAAGCTCAAGAGCGTGAAGGAGCTCCTGGGGATC | |
| | ACCATCATGGAGAGGTCCAGCTTCGAGAAGAACCCAATCGACTTCCTGGAG | |
| | GCCAAGGGCTACAAGGAGGTGAAGAAGGACCTGATCATCAAGCTCCCGAAG | |
| | TACTCTCTCTTCGAGCTGGAGAACGGCAGGAAGAGAATGCTGGCTTCCGCT | |
| | GGCGAGCTCCAGAAGGGGAACGAGCTCGCGCTGCCAAGCAAGTACGTGAAC | |
| | TTCCTCTACCTGGCTTCCCACTACGAGAAGCTCAAGGGCAGCCCGGAGGAC | |
| | AACGAGCAAAAGCAGCTGTTCGTCGAGCAGCACAAGCATTACCTCGACGAG | |
| | ATCATCGAGCAAATCTCCGAGTTCAGCAAGCGCGTGATCCTCGCCGACGCG | |
| | AACCTGGATAAGGTCCTCTCCGCCTACAACAAGCACCGGGACAAGCCCATC | |
| | AGAGAGCAAGCGGAGAACATCATCCATCTCTTCACCCTGACGAACCTCGGC | |
| | GCTCCTGCTGCTTTCAAGTACTTCGACACCACGATCGATCGGAAGAGATAC | |
| | ACCTCCACGAAGGAGGTCCTGGACGCGACCCTCATCCACCAGTCGATCACC | |
| | GGCCTGTACGAGACGAGGATCGACCTCTCACAACTCGGCGGGGATAAGAGA | |
| | CCCGCAGCAACCAAGAAGGCAGGGCAAGCAAAGAAGAAGAAGTGActcgag | |
| | tccctagagtCCTGCTTTAATGAGATATGCGAGACGCCTATGATCGCATGA | |
| | TATTTGCTTTCAATTCTGTTGTGCACGTTGTAAAAAACCTGAGCATGTGTA | |
| | GCTCAGATCCTTACCGCCGGTTTCGGTTCATTCTAATGAATATATCACCCG | |
| | TTACTATCGTATTTTTATGAATAATATTCTCCGTTCAATTTACTGATTGTA | |
| | CCCTACTACTTATATGTACAATATTAAAATGAAAACAATATATTGTGCTGA | |
| | ATAGGTTTATAGCGACATCTATGATAGAGCGCCACAATAACAAACAATTGC | |
| | GTTTTATTATTACAAATCCAATTTTAAAAAAAGCGGCAGAACCGGTCAAAC | |
| | CTAAAAGACTGATTACATAAATCTTATTCAAATTTCAAAAGTGCCCGGGCT | |
| | AGTATCTACGACACACCGAGCGGCGAACTAATAACGCTCACTGAAGGGAAC | |
| | TCCGGTTCCCCGCCGGCGCGCATGGGTGAGATTCCTTGAAGTTGAGTATTG | |
| | GCCGTCCGCTCTACCGAAAGTTACGGGCACCATTCAACCCGGTCCAGCACG | |
| | GCGGCCGGGTAACCGACTTGCTGCCCCGAGAATTATGCAGCATTTTTTTGG | |
| | TGTATGTGGGCCCCAAATGAAGTGCAGGTCAAACCTTGACAGTGACGACAA | |
| | ATCGTTGGGCGGGTCCAGGGCGAATTTTGCGACAACATGTCGAGGCTCAGC | |
| | AGGAggacGACCAAGCCCGTTATTCTGACAGTTCTGGTGCTCAACACATTT | |
| | ATATTTATCAAGGAGCACATTGTTACTCACTGCTAGGAGGGAATCGAACTA | |
| | GGAATATTGATCAGAGGAACTACGAGAGAGCTGAAGATAACTGCCCTCTAG | |
| | CTCTCACTGATCTGGGTCGCATAGTGAGATGCAGCCCACGTGAGTTCAGCA | |
| | ACGGTCTAGCGCTGGGCTTTTAGGCCCGCATGATCGGGCTTTTGTCGGGTG | |
| | GTCGACGTGTTCACGATTGGGGAGAGCAACGCAGCAGTTCCTCTTAGTTTA | |
| | GTCCCACCTCGCCTGTCCAGCAGAGTTCTGACCGGTTTATAAACTCGCTTG | |
| | CTGCATCAGacttGGCCCGGACTTCGAGCCAATGTTTTAGAGCTAGAAATA | |
| | GCAAGTTAAAATAAGGCTAGTCCGTTATCAACTTGAAAAAGTGGCACCGAG | |
| | TCGGTGCTTTTTTTccggGGCGGCAGGGAGAGTTTTAACATTGACTAGCGT | |
| | GCTGATAATTTGTGAGAAATAATAATTGACAAGTAGATACTGACATTTGAG | |
| | AAGAGCTTCTGAACTGTTATTAGTAACAAAAATGGAAAGCTGATGCACGGA | |
| | AAAAGGAAAGAAAAAGCCATACTTTTTTTTAGGTAGGAAAAGAAAAAGCCA | |
| | TACGAGACTGATGTCTCTCAGATGGGCCGGGATCTGTCTATCTAGCAGGCA | |
| | GCAGCCCTACCAACCTCACGGGCCAGCAATTACGAGTCCTTCTAAAACGTC | |
| | CCGCCGAGGGCGCGTGGCCGTGCTGTGCAGCAGCACGTCTAACATTAGTCC | |
| | CACCTCGCCAGTTTACAGGGAGCAGAACCAGCTTATAAGCGGAGGCGCGGC | |
| | ACCAAgaagGCGACTTCTTCGAGGACAGCGTTTTAGAGCTAGAAATAGCAA | |
| | GTTAAAATAAGGCTAGTCCGTTATCAACTTGAAAAAGTGGCACCGAGTCGG | |
| | TGCTTTTTTT | |
| 40 | TGCAGTGCAGCGTGACCCGGTCGTGCCCCTCTCTAGAGATAATGAGCATTG | Cas9 and |
| | CATGTCTAAGTTATAAAAAATTACCACATATTTTTTTTGTCACACTTGTTT | Eragrostis |
| | GAAGTGCAGTTTATCTATCTTTATACATATATTTAAACTTTACTCTACGAA | tef DW3 and |
| | TAATATAATCTATAGTACTACAATAATATCAGTGTTTTAGAGAATCATATA | DW1 gRNA |
| | AATGAACAGTTAGACATGGTCTAAAGGACAATTGAGTATTTTGACAACAGG | expression |
| | ACTCTACAGTTTTATCTTTTTAGTGTGCATGTGTTCTCCTTTTTTTTTGCA | cassettes |
| | AATAGCTTCACCTATATAATACTTCATCCATTTTATTAGTACATCCATTTA | used to |
| | GGGTTTAGGGTTAATGGTTTTTATAGACTAATTTTTTTAGTACATCTATTT | generate |
| | TATTCTATTTTAGCCTCTAAATTAAGAAAACTAAAACTCTATTTTAGTTTT | semidwarf |
| | TTTATTTAATAATTTAGATATAAAATAGAATAAAATAAAGTGACTAAAAAT | tef line |
| | TAAACAAATACCCTTTAAGAAATTAAAAAAACTAAGGAAACATTTTTCTTG | >1-982 |
| | TTTCGAGTAGATAATGCCAGCCTGTTAAACGCCGTCGACGAGTCTAACGGA | (Maize |
| | CACCAACCAGCGAACCAGCAGCGTCGCGTCGGGCCAAGCGAAGCAGACGGC | Ubiquitin-1 |
| | ACGGCATCTCTGTCGCTGCCTCTGGACCCCTCTCGAGAGTTCCGCTCCACC | promoter) |
| | GTTGGACTTGCTCCGCTGTCGGCATCCAGAAATTGCGTGGCGGAGCGGCAG | >983-1995 |
| | ACGTGAGCCGGCACGGCAGGCGGCCTCCTCCTCCTCTCACGGCACCGGCAG | (Maize |
| | CTACGGGGGATTCCTTTCCCACCGCTCCTTCGCTTTCCCTTCCTCGCCCGC | Ubiquitin-1, |
| | CGTAATAAATAGACACCCCCTCCACACCCTCTTTCCCCAACCTCGTGTTGT | intron-1) |
| | TCGGAGCGCACACACACACAACCAGATCTCCCCCAAATCCACCCGTCGGCA | >1996-2010 |
| | CCTCCGCTTCAAGGTACGCCGCTCGTCCTCCCCCCCCCCCCTCTCTACCTT | (intervening |
| | CTCTAGATCGGCGTTCCGGTCCATGGTTAGGGCCCGGTAGTTCTACTTCTG | sequence) |
| | TTCATGTTTGTGTTAGATCCGTGTTTGTGTTAGATCCGTGCTGCTAGCGTT | >2011-6165 |
| | CGTACACGGATGCGACCTGTACGTCAGACACGTTCTGATTGCTAACTTGCC | (Cas9 coding |
| | AGTGTTTCTCTTTGGGGAATCCTGGGATGGCTCTAGCCGTTCCGCAGACGG | sequence) |
| | GATCGATTTCATGATTTTTTTTGTTTCGTTGCATAGGGTTTGGTTTGCCCT | >6166-6181 |
| | TTTCCTTTATTTCAATATATGCCGTGCACTTGTTTGTCGGGTCATCTTTTC | (intervening |
| | ATGCTTTTTTTTGTCTTGGTTGTGATGATGTGGTCTGGTTGGGCGGTCGTT | sequence) |
| | CTAGATCGGAGTAGAATTAATTCTGTTTCAAACTACCTGGTGGATTTATTA | >6182-6889 |
| | ATTTTGGATCTGTATGTGTGTGCCATACATATTCATAGTTACGAATTGAAG | (Octapine |
| | ATGATGGATGGAAATATCGATCTAGGATAGGTATACATGTTGATGCGGGTT | synthase |
| | TTACTGATGCATATACAGAGATGCTTTTTGTTCGCTTGGTTGTGATGATGT | polyA |
| | GGTGTGGTTGGGCGGTCGTTCATTCGTTCTAGATCGGAGTAGAATACTGTT | signal) |
| | TCAAACTACCTGGTGTATTTATTAATTTTGGAACTGTATGTGTGTGTCATA | >6890-6893 |
| | CATCTTCATAGTTACGAGTTTAAGATGGATGGAAATATCGATCTAGGATAG | (intervening |
| | GTATACATGTTGATGTGGGTTTTACTGATGCATATACATGATGGCATATGC | sequence) |
| | AGCATCTATTCATATGCTCTAACCTTGAGTACCTATCTATTATAATAAACA | >6894-7251 |
| | AGTATGTTTTATAATTATTTTGATCTTGATATACTTGGATGATGGCATATG | (Triticum |
| | CAGCAGCTATATGTGGATTTTTTTAGCCCTGCCTTCATACGCTATTTATTT | aestivum U6 |
| | GCTTGGTACTGTTTCTTTTGTCGATGCTCACCCTGTTGTTTGGTGTTACTT | small |
| | CCTGCAgggcgatctattcgaATGGACAAGAAGTACTCGATCGGCCTCGAC | nuclear RNA |
| | ATCGGGACGAACTCAGTTGGCTGGGCCGTGATCACCGACGAGTACAAGGTG | gene |
| | CCCTCTAAGAAGTTCAAGGTCCTGGGGAACACCGACCGCCATTCCATCAAG | promoter) |
| | AAGAACCTCATCGGCGCTCTCCTGTTCGACAGCGGGGAGACCGCTGAGGCT | >7252-7256 |
| | ACGAGGCTCAAGAGAACCGCTAGGCGCCGGTACACGAGAAGGAAGAACAGG | (intervening |
| | ATCTGCTACCTCCAAGAGATTTTCTCCAACGAGATGGCCAAGGTTGACGAT | sequence) |
| | TCATTCTTCCACCGCCTGGAGGAGTCTTTCCTCGTGGAGGAGGATAAGAAG | >7256-7275 |
| | CACGAGCGGCATCCCATCTTCGGCAACATCGTGGACGAGGTTGCCTACCAC | (Eragrostis |
| | GAGAAGTACCCTACGATCTACCATCTGCGGAAGAAGCTCGTGGACTCCACC | tef DW3- |
| | GATAAGGCGGACCTCAGACTGATCTACCTCGCTCTGGCCCACATGATCAAG | gRNA) |
| | TTCCGCGGCCATTTCCTGATCGAGGGGGATCTCAACCCAGACAACAGCGAT | >7277-7359 |
| | GTTGACAAGCTGTTCATCCAACTCGTGCAGACCTACAACCAACTCTTCGAG | (gRNA |
| | GAGAACCCGATCAACGCCTCTGGCGTGGACGCGAAGGCTATCCTGTCCGCG | scaffold) |
| | AGGCTCTCGAAGTCCAGGAGGCTGGAGAACCTGATCGCTCAGCTCCCAGGC | >7360-7363 |
| | GAGAAGAAGAACGGCCTGTTCGGGAACCTCATCGCTCTCAGCCTGGGGCTC | (intervening |
| | ACCCCGAACTTCAAGTCGAACTTCGATCTCGCTGAGGACGCCAAGCTGCAA | sequence) |
| | CTCTCCAAGGACACCTACGACGATGACCTCGATAACCTCCTGGCCCAGATC | 7364-7758 |
| | GGCGATCAATACGCGGACCTGTTCCTCGCTGCCAAGAACCTGTCGGACGCC | (Triticum |
| | ATCCTCCTGTCAGATATCCTCCGCGTGAACACCGAGATCACGAAGGCTCCA | aestivum U3 |
| | CTCTCTGCCTCCATGATCAAGCGCTACGACGAGCACCATCAGGATCTGACC | small |
| | CTCCTGAAGGCGCTGGTCCGCCAACAGCTCCCGGAGAAGTACAAGGAGATT | nuclear RNA |
| | TTCTTCGATCAGTCGAAGAACGGCTACGCTGGGTACATCGACGGCGGGGCC | gene |
| | TCACAAGAGGAGTTCTACAAGTTCATCAAGCCAATCCTGGAGAAGATGGAC | promoter) |
| | GGCACGGAGGAGCTCCTGGTGAAGCTCAACAGGGAGGACCTCCTGCGGAAG | >7759-7763 |
| | CAGAGAACCTTCGATAACGGCAGCATCCCCCACCAAATCCATCTCGGGGAG | (intervening |
| | CTGCACGCCATCCTGAGAAGGCAAGAGGACTTCTACCCTTTCCTCAAGGAT | sequence) |
| | AACCGGGAGAAGATCGAGAAGATCCTGACCTTCAGAATCCCATACTACGTC | >7764-7783 |
| | GGCCCTCTCGCGCGGGGGAACTCAAGATTCGCTTGGATGACCCGCAAGTCT | (Eragrostis |
| | GAGGAGACCATCACGCCGTGGAACTTCGAGGAGGTGGTGGACAAGGGCGCT | tef DW1- |
| | AGCGCTCAGTCGTTCATCGAGAGGATGACCAACTTCGACAAGAACCTGCCC | gRNA) |
| | AACGAGAAGGTGCTCCCTAAGCACTCGCTCCTGTACGAGTACTTCACCGTC | >7784-7866 |
| | TACAACGAGCTCACGAAGGTGAAGTACGTCACCGAGGGCATGCGCAAGCCA | (gRNA |
| | GCGTTCCTGTCCGGGGAGCAGAAGAAGGCTATCGTGGACCTCCTGTTCAAG | scaffold) |
| | ACCAACCGGAAGGTCACGGTTAAGCAACTCAAGGAGGACTACTTCAAGAAG | |
| | ATCGAGTGCTTCGATTCGGTCGAGATCAGCGGCGTTGAGGACCGCTTCAAC | |
| | GCCAGCCTCGGGACCTACCACGATCTCCTGAAGATCATCAAGGATAAGGAC | |
| | TTCCTGGACAACGAGGAGAACGAGGATATCCTGGAGGACATCGTGCTGACC | |
| | CTCACGCTGTTCGAGGACAGGGAGATGATCGAGGAGCGCCTGAAGACGTAC | |
| | GCCCATCTCTTCGATGACAAGGTCATGAAGCAACTCAAGCGCCGGAGATAC | |
| | ACCGGCTGGGGGAGGCTGTCCCGCAAGCTCATCAACGGCATCCGGGACAAG | |
| | CAGTCCGGGAAGACCATCCTCGACTTCCTCAAGAGCGATGGCTTCGCCAAC | |
| | AGGAACTTCATGCAACTGATCCACGATGACAGCCTCACCTTCAAGGAGGAT | |
| | ATCCAAAAGGCTCAAGTGAGCGGCCAGGGGGACTCGCTGCACGAGCATATC | |
| | GCGAACCTCGCTGGCTCCCCCGCGATCAAGAAGGGCATCCTCCAGACCGTG | |
| | AAGGTTGTGGACGAGCTCGTGAAGGTCATGGGCCGGCACAAGCCTGAGAAC | |
| | ATCGTCATCGAGATGGCCAGAGAGAACCAAACCACGCAGAAGGGGCAAAAG | |
| | AACTCTAGGGAGCGCATGAAGCGCATCGAGGAGGGCATCAAGGAGCTGGGG | |
| | TCCCAAATCCTCAAGGAGCACCCAGTGGAGAACACCCAACTGCAGAACGAG | |
| | AAGCTCTACCTGTACTACCTCCAGAACGGCAGGGATATGTACGTGGACCAA | |
| | GAGCTGGATATCAACCGCCTCAGCGATTACGACGTCGATCATATCGTTCCC | |
| | CAGTCTTTCCTGAAGGATGACTCCATCGACAACAAGGTCCTCACCAGGTCG | |
| | GACAAGAACCGCGGCAAGTCAGATAACGTTCCATCTGAGGAGGTCGTTAAG | |
| | AAGATGAAGAACTACTGGAGGCAGCTCCTGAACGCCAAGCTGATCACGCAA | |
| | AGGAAGTTCGACAACCTCACCAAGGCTGAGAGAGGCGGGCTCTCAGAGCTG | |
| | GACAAGGCCGGCTTCATCAAGCGGCAGCTGGTCGAGACCAGACAAATCACG | |
| | AAGCACGTTGCGCAAATCCTCGACTCTCGGATGAACACGAAGTACGATGAG | |
| | AACGACAAGCTGATCAGGGAGGTTAAGGTGATCACCCTGAAGTCTAAGCTC | |
| | GTCTCCGACTTCAGGAAGGATTTCCAGTTCTACAAGGTTCGCGAGATCAAC | |
| | AACTACCACCATGCCCATGACGCTTACCTCAACGCTGTGGTCGGCACCGCT | |
| | CTGATCAAGAAGTACCCAAAGCTGGAGTCCGAGTTCGTGTACGGGGACTAC | |
| | AAGGTTTACGATGTGCGCAAGATGATCGCCAAGTCGGAGCAAGAGATCGGC | |
| | AAGGCTACCGCCAAGTACTTCTTCTACTCAAACATCATGAACTTCTTCAAG | |
| | ACCGAGATCACGCTGGCCAACGGCGAGATCCGGAAGAGACCGCTCATCGAG | |
| | ACCAACGGCGAGACGGGGGAGATCGTGTGGGACAAGGGCAGGGATTTCGCG | |
| | ACCGTCCGCAAGGTTCTCTCCATGCCCCAGGTGAACATCGTCAAGAAGACC | |
| | GAGGTCCAAACGGGGGGTTCTCAAAGGAGTCTATCCTGCCTAAGCGGAAC | |
| | AGCGACAAGCTCATCGCCAGAAAGAAGGACTGGGACCCAAAGAAGTACGGC | |
| | GGGTTCGACAGCCCTACCGTGGCCTACTCGGTCCTGGTTGTGGCGAAGGTT | |
| | GAGAAGGGCAAGTCCAAGAAGCTCAAGAGCGTGAAGGAGCTCCTGGGGATC | |
| | ACCATCATGGAGAGGTCCAGCTTCGAGAAGAACCCAATCGACTTCCTGGAG | |
| | GCCAAGGGCTACAAGGAGGTGAAGAAGGACCTGATCATCAAGCTCCCGAAG | |
| | TACTCTCTCTTCGAGCTGGAGAACGGCAGGAAGAGAATGCTGGCTTCCGCT | |
| | GGCGAGCTCCAGAAGGGGAACGAGCTCGCGCTGCCAAGCAAGTACGTGAAC | |
| | TTCCTCTACCTGGCTTCCCACTACGAGAAGCTCAAGGGCAGCCCGGAGGAC | |
| | AACGAGCAAAAGCAGCTGTTCGTCGAGCAGCACAAGCATTACCTCGACGAG | |
| | ATCATCGAGCAAATCTCCGAGTTCAGCAAGCGCGTGATCCTCGCCGACGCG | |
| | AACCTGGATAAGGTCCTCTCCGCCTACAACAAGCACCGGGACAAGCCCATC | |
| | AGAGAGCAAGCGGAGAACATCATCCATCTCTTCACCCTGACGAACCTCGGC | |
| | GCTCCTGCTGCTTTCAAGTACTTCGACACCACGATCGATCGGAAGAGATAC | |
| | ACCTCCACGAAGGAGGTCCTGGACGCGACCCTCATCCACCAGTCGATCACC | |
| | GGCCTGTACGAGACGAGGATCGACCTCTCACAACTCGGCGGGGATAAGAGA | |
| | CCCGCAGCAACCAAGAAGGCAGGGCAAGCAAAGAAGAAGAAGTGActcgag | |
| | tccctagagtCCTGCTTTAATGAGATATGCGAGACGCCTATGATCGCATGA | |
| | TATTTGCTTTCAATTCTGTTGTGCACGTTGTAAAAAACCTGAGCATGTGTA | |
| | GCTCAGATCCTTACCGCCGGTTTCGGTTCATTCTAATGAATATATCACCCG | |
| | TTACTATCGTATTTTTATGAATAATATTCTCCGTTCAATTTACTGATTGTA | |
| | CCCTACTACTTATATGTACAATATTAAAATGAAAACAATATATTGTGCTGA | |
| | ATAGGTTTATAGCGACATCTATGATAGAGCGCCACAATAACAAACAATTGC | |
| | GTTTTATTATTACAAATCCAATTTTAAAAAAAGCGGCAGAACCGGTCAAAC | |
| | CTAAAAGACTGATTACATAAATCTTATTCAAATTTCAAAAGTGCCCGGGCT | |
| | AGTATCTACGACACACCGAGCGGCGAACTAATAACGCTCACTGAAGGGAAC | |
| | TCCGGTTCCCCGCCGGCGCGCATGGGTGAGATTCCTTGAAGTTGAGTATTG | |
| | GCCGTCCGCTCTACCGAAAGTTACGGGCACCATTCAACCCGGTCCAGCACG | |
| | GCGGCCGGGTAACCGACTTGCTGCCCCGAGAATTATGCAGCATTTTTTTGG | |
| | TGTATGTGGGCCCCAAATGAAGTGCAGGTCAAACCTTGACAGTGACGACAA | |
| | ATCGTTGGGCGGGTCCAGGGCGAATTTTGCGACAACATGTCGAGGCTCAGC | |
| | AGGAggacGACCAAGCCCGTTATTCTGACAGTTCTGGTGCTCAACACATTT | |
| | ATATTTATCAAGGAGCACATTGTTACTCACTGCTAGGAGGGAATCGAACTA | |
| | GGAATATTGATCAGAGGAACTACGAGAGAGCTGAAGATAACTGCCCTCTAG | |
| | CTCTCACTGATCTGGGTCGCATAGTGAGATGCAGCCCACGTGAGTTCAGCA | |
| | ACGGTCTAGCGCTGGGCTTTTAGGCCCGCATGATCGGGCTTTTGTCGGGTG | |
| | GTCGACGTGTTCACGATTGGGGAGAGCAACGCAGCAGTTCCTCTTAGTTTA | |
| | GTCCCACCTCGCCTGTCCAGCAGAGTTCTGACCGGTTTATAAACTCGCTTG | |
| | CTGCATCAGacttGTCGTGCGCCCGATCATGAACGTTTTAGAGCTAGAAAT | |
| | AGCAAGTTAAAATAAGGCTAGTCCGTTATCAACTTGAAAAAGTGGCACCGA | |
| | GTCGGTGCTTTTTTTccggGGCGGCAGGGAGAGTTTTAACATTGACTAGCG | |
| | TGCTGATAATTTGTGAGAAATAATAATTGACAAGTAGATACTGACATTTGA | |
| | GAAGAGCTTCTGAACTGTTATTAGTAACAAAAATGGAAAGCTGATGCACGG | |
| | AAAAAGGAAAGAAAAAGCCATACTTTTTTTTAGGTAGGAAAAGAAAAAGCC | |
| | ATACGAGACTGATGTCTCTCAGATGGGCCGGGATCTGTCTATCTAGCAGGC | |
| | AGCAGCCCTACCAACCTCACGGGCCAGCAATTACGAGTCCTTCTAAAACGT | |
| | CCCGCCGAGGGCGCGTGGCCGTGCTGTGCAGCAGCACGTCTAACATTAGTC | |
| | CCACCTCGCCAGTTTACAGGGAGCAGAACCAGCTTATAAGCGGAGGCGCGG | |
| | CACCAAgaagGCCTGGAATATGGTGTGTCACGTTTTAGAGCTAGAAATAGC | |
| | AAGTTAAAATAAGGCTAGTCCGTTATCAACTTGAAAAAGTGGCACCGAGTC | |
| | GGTGCTTTTTTT | |

### EXAMPLES

All patents and publications mentioned in the specification are indicative of the levels of those skilled in the art to which the present disclosure pertains. All patents and publications are herein incorporated by reference to the same extent as if each individual publication was specifically and individually indicated to be incorporated by reference.

The publications discussed throughout are provided solely for their disclosure before the filing date of the present application. Nothing herein is to be construed as an admission that the invention is not entitled to antedate such disclosure by virtue of prior invention.

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense.

### Example 1. Development of Reduced Plant Height Teff

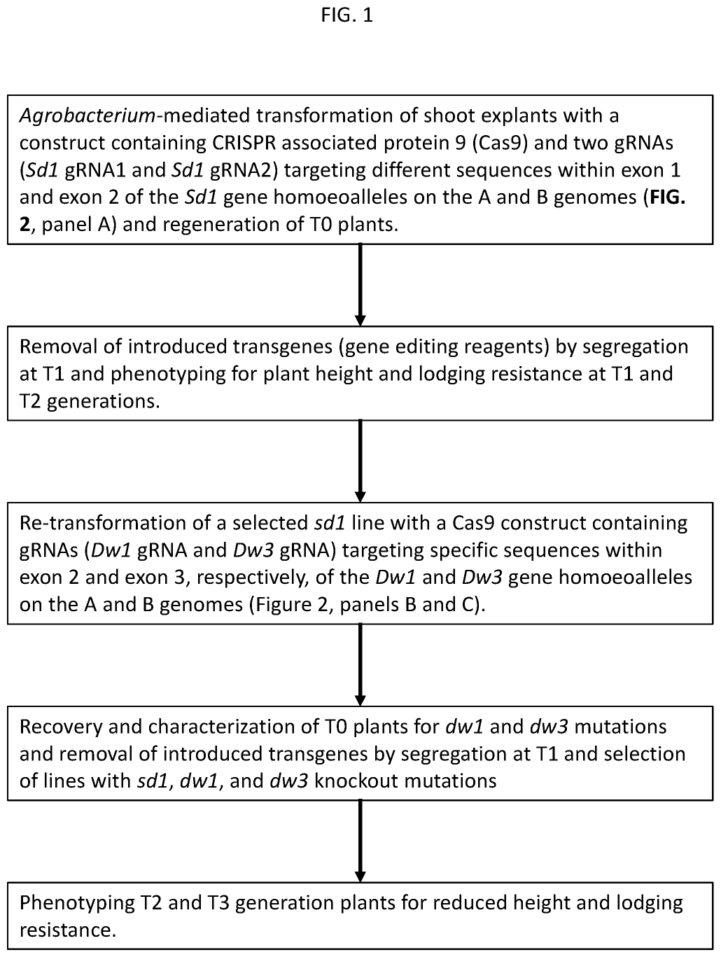

The development of reduced plant height DPS 22-8-12 teff followed a step-wise process that included two plant transformation steps, one to introduce targeted mutations within the Sd1 gene and a re-transformation of a sd1 line to simultaneously introduce targeted mutations within the Dw1 and Dw3 genes (FIG. 1).

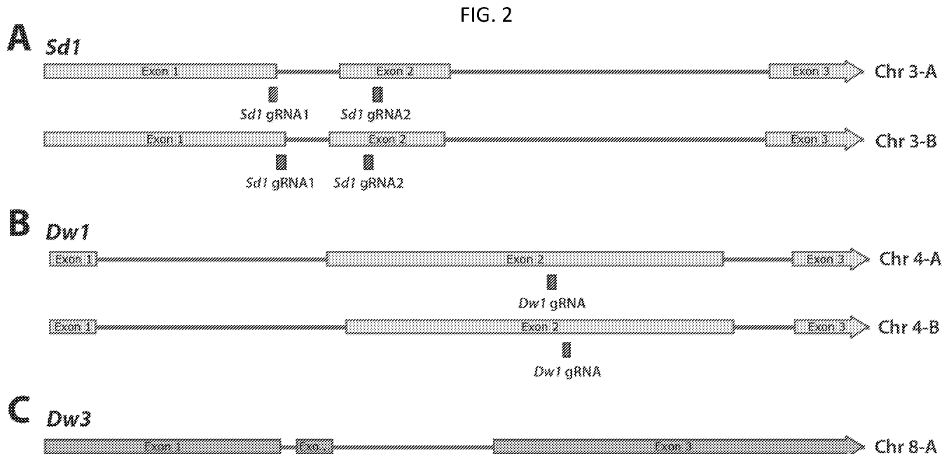

Sequences of rice (Oryza sativa L.) GA20-oxidases Sd1 (OsGA20ox1-OsGA20ox4), sorghum [Sorghum bicolor L. (Moench)] Dw1 (Sobic.009G229800, Chr09:57093313 . . . 57095643) and Dw3 (Sobic.007G163800, Chr07:59821905 . . . 59829910) were used as baits to retrieve orthologous sequences from the published teff genome using CoGeBlast, which were then used to design specific gRNAs. The nucleotide sequences of the gRNAs targeting the teff Sd1, Dw1, and Dw3 genes are shown in Table 1 and their relative locations on the target genes are illustrated schematically in FIG. 2.

TABLE 1
Guide RNA target sequences

| | Target | | | |
|---|---|---|---|---|
| | Gene | Chromosome | | |
| Name | (Exon) | Location¹ | Strand | Sequence² |
| Sd1 | Sd1 | 3A | Sense | 5′-GGCCC |
| gRNA1 | (Exon | (31096526..31096548) | | GGACTTCG |
| | 1) | 3B | | AGCCAATG |
| | | (29293138..29293160) | | GG-3′ |
| Sd1 | Sd1 | 3A | Sense | 5′-GCGAC |
| gRNA2 | (Exon | (31096827..31096849) | | TTCTTCGA |
| | 2) | 3B | | GGACAGCC |
| | | (29293380..29293402) | | GG-3′ |
| Dw1 | Dw1 | 4A | Anti- | 5′-CCTGG |
| gRNA | (Exon | (7977763..7977785) | sense | AATATGGT |
| | 2) | 4B | | GTGTCACA |
| | | (7681439..7681461) | | GG-3′ |
| Dw3 | Dw3 | 8A | Anti- | 5′-TCGTG |
| gRNA | (Exon | (145900..145878) | sense | CGCCCGAT |
| | 3) | 8B | | CATGAACC |
| | | (134813..134791) | | GG-3′ |

¹Chromosome locations are relative to the E. teff database (id 50954 PacBio unmasked vV3) BLAST searches against the teff genome database using gRNA sequences as queries.
²Underlined nucleotides correspond to the protospacer adjacent motif (PAM) recognition sites.

Null-segregant plant lines containing targeted mutations without introduced foreign DNA were selected at the T1 generation using qPCR analysis and NGS was used to confirm the identity of the knockout mutations.

### Example 2. Information on the Comparator Plant

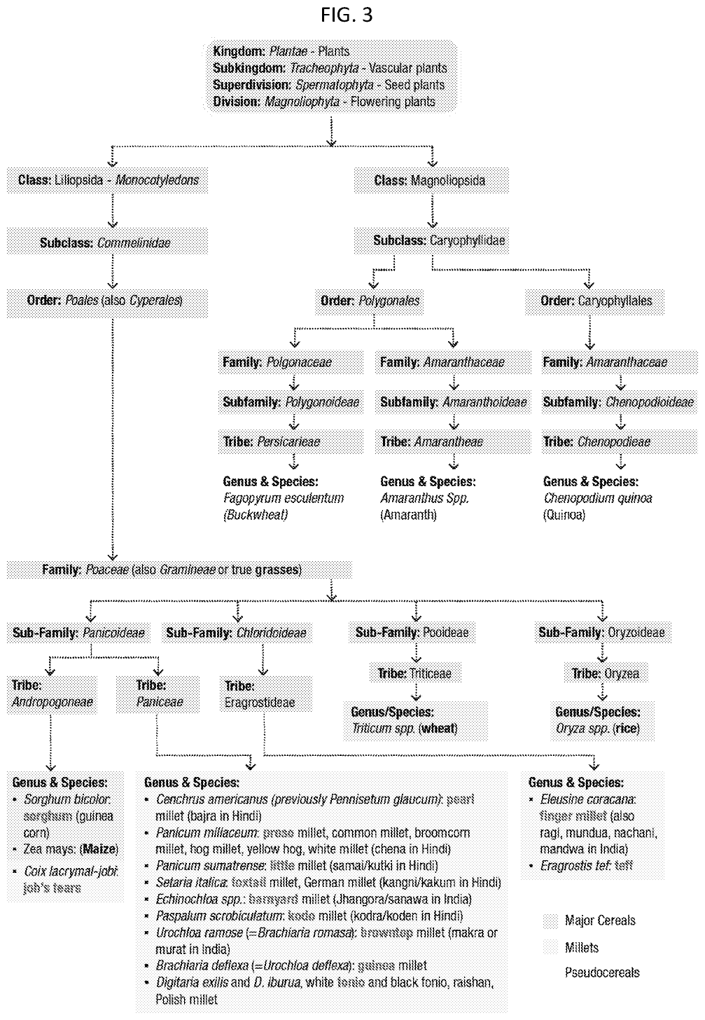

Teff [Eragrostis teff (Zucc.) Trotter] is an allotetraploid (2n=4×=40) C4 plant belonging to the Poaceae or Grass family and is closely related to finger millet (Eleusine coracana Gaerth.) as both are in the subfamily Chloridoideae (FIG. 3). The genus Eragrostis comprises about 350 species from which only teff is cultivated for human consumption.

Ethiopia is the origin and center of diversity for teff. Although the exact date and location of the domestication of teff is unknown, it is believed to be between 1000-4000 BC.

Various investigators have speculated on the origins of teff using morphological, cytological, and/or biochemical characterizations and have suggested a total of 14 wild Eragrostis species as potential progenitors of the crop. The general consensus is that the wild progenitor of teff is likely Eragrostis pilosa, a hardy wild grass sharing considerable overlap in morphological, genetic, and karyotype traits with teff. Eragrostis pilosa is also an allotetraploid native to Eurasia and Africa, and occurs as a common weed throughout the world in tropical and temperate regions. Other Eragrostis species thought to be involved in the evolution of teff include E. heteromera, E. aethiopica, E. barrelieri, E. curvula, and E. cilianensis.

It is likely that both E. pilosa and teff arose from a shared polyploidy event that merged two currently unknown and possibly extinct or unsampled diploid genomes. The only documented and consistent morphological distinction between E. pilosa and teff is spikelet shattering. The multi-floreted spikelets of E. pilosa readily break apart at maturity as a natural mechanism of seed dispersal, whereas the lemmas, paleas, and caryopses of teff remain attached to the rachis at maturity and thereby facilitate harvesting. It has been hypothesized that teff is a domesticate of E. pilosa in which several key agronomic features (e.g., seed mass and spikelet shattering) have been altered through generations of human selection.

The flowers of teff are hermaphroditic with both the stamens and pistils being found in the same floret. The florets consist of a lemma, palea, three stamens, two stigma, and two lodicules that assist in flower opening. Panicle spikelets consist of 2-12 florets, and floret color may vary from white to dark brown. Teff is a self pollinated chasmogamous plant and the degree of outcrossing is very low, ranging between 0.2-1 percent. Teff flowers open and pollinate for only a short time in the early morning hours, typically between 6:45 and 7:45 AM under Ethiopian climatic conditions.

Teff germplasm contains a wealth of diversity that offers many opportunities for genetic improvement and the development of new varieties suitable for various agro-ecosystems, cropping practices, and end uses. Of relevance, there is significant variability in plant height. Teff plants grow to a total height ranging from about 20 to 155 cm, of which the culm (11-82 cm) and the panicle (10-65 cm) comprise 47-65 percent and 35-56 percent, respectively (Table 2). Improved teff cultivars tend to be tall in plant height, which may be due to selection for higher grain yield alone and this may aggravate lodging severity as it does in most cereal crops.

TABLE 2
Ranges of important traits of teff

| | | |
|---|---|---|
| Traits¹ | Minimum | Maximum |

Time-related

| | | |
|---|---|---|
| Days to heading | 25 | 81 |
| Days to maturity | 60 | 140 |
| Grain filling period (days) | 29 | 75 |

Length-related

| | | |
|---|---|---|
| Plant height (cm) | 20 | 156 |
| Culm length (cm) | 11 | 82 |
| First culm internode length (cm) | 2.7 | 8.1 |
| Second culm internode length (cm) | 4.1 | 11.5 |
| Panicle length (cm) | 10 | 66 |
| Peduncle length (cm) | 5.8 | 42.3 |

Diameter-related

| | | |
|---|---|---|
| First and second culm internode diameter (mm) | 1.2 | 4.5 |
| Grain diameter (mm) | 0.5 | 1 |

Number-related

| | | |
|---|---|---|
| Total tillers per plant | 4 | 22 |
| Fertile tillers per plant | 1 | 17 |
| Primary panicle branches per plant | 10 | 40 |
| Spikelets per panicle | 30 | 1070 |
| Florets per spikelet | 3 | 17 |

Mass- and yield-related

| | | |
|---|---|---|
| 100-kernel mass (mg) | 18.9 | 33.9 |
| Grain yield per panicle (g) | 0.1 | 2.5 |
| Grain yield per plant (g) | 0.5 | 21.9 |
| Bulk grain yield (kg/ha) | 1058 | 4599 |
| Total biomass per plant (g) | 4 | 105 |
| Shoot biomass (kg/ha) | 6355 | 19630 |

Other properties

| | | |
|---|---|---|
| Harvest index (%) | 5 | 39 |
| Lodging index (%) | 20 | 100 |

¹Reproduced from Assefa et al. (2017).

### Example 3. Genotypes, and Phenotypes of Genetically Modified Plants

Mutations in the teff SD1, DW1, and DW3 genes were generated using clustered regularly interspersed short palindromic repeats (CRISPR)-Cas9 targeted mutation. The sd1, dw1, dw3 mutations were limited to single nucleotide insertions (A or T) or dinucleotide (GC) deletions (Table 3). The mutations were characterized by nucleotide sequencing of the target loci in T1 generation progeny plants that had been confirmed free of introduced foreign DNA by qPCR. Line DPS 22-8-12 was one of the lines identified that was homozygous at each homoeoallele of the Sd1, Dw1, and Dw3 genes for a knockout (loss-of-function) mutation.

TABLE 3
Summary of sd1, dw1, and dw3 mutations in DPS 22-8-12 teff

| Alleleª | gRNA1 Target Sequence^{b} | Mutation | gRNA2 Target Sequence | Mutation | CDS^{c} | Comments |
|---|---|---|---|---|---|---|
| | | | | | | SD1 (A |
| | | | | | | Wild-type |
| Sd1A | GGCCCGGACTTCGAGCCAAT | | GCGACTTCTTCGAGGACAGC | | 420 | genome) |
| sd1A1 | GGCCCGGACTTCGA--CAAT | -GC | GCGACTTCTTCGAGGACAAGC | +A | 347 | Altered |
| sd1A2 | GGCCCGGACTTCGA--CAAT | -GC | GCGACTTCTTCGAGGACAAGC | +A | | amino acid |
| | | | | | | sequence |
| | | | | | | after F221 |
| | | | | | | Wild-type |
| | | | | | | SD1 (B |
| Sd1B | GGCCCGGACTTCGAGCCAAT | | GCGACTTCTTCGAGGACAGC | | 419 | genome) |
| sd1B1 | GGCCCGGACTTCGAGCCAAAT | +A | GCGACTTCTTCGAGGACAGC | — | 245 | Altered |
| sd1B2 | GGCCCGGACTTCGAGCCAAAT | +A | GCGACTTCTTCGAGGACAGC | — | | amino acid |
| | | | | | | sequence |
| | | | | | | after P222 |

| Allele | gRNA Target Sequence^{b} | Mutation | CDS | Comments |
|---|---|---|---|---|
| Dw1A | GTGACACACCATATTCCAGG | | 528 | Wild-type DW1 (A Genome) |
| dw1A1 | GTGAACACACCATATTCCAGG | +A | 287 | Altered amino acid sequence after |
| dw1A2 | GTGAACACACCATATTCCAGG | +A | | C276 and premature termination |
| Dw1B | GTGACACACCATATTCCAGG | | 528 | Wild-type DW1 (B Genome) |
| dw1B1 | GTGAACACACCATATTCCAGG | +A | 287 | Altered amino acid sequence after |
| dw1B2 | GTGAACACACCATATTCCAGG | +A | | C276 and premature termination |
| Dw3A | GTTCATGATCGGGCGCACGA | | 1354 | Wild-type DW3 (A Genome) |
| dw3A1 | GTTTCATGATCGGGCGCACGA | +T | 743 | Altered amino acid sequence after |
| dw3A2 | GTTTCATGATCGGGCGCACGA | +T | | F630 and premature termination |
| Dw3B | GTTCATGATCGGGCGCACGA | | 1347 | Wild-type DW3 (B Genome) |
| dw3B1 | GTTACATGATCGGGCGCACGA | +A | 736 | Altered amino acid sequence after |
| dw3B2 | GTTACATGATCGGGCGCACGA | +A | | R620 and premature termination |

^{a}The naming convention for wild-type alleles uses three-letter codes for each gene with the first letter capitalized (e.g., Sd1, Dw1, and Dw3) followed by the genome designation, A or B. Mutant alleles are identified with lowercase three-letter codes, followed by the genome designation (A or B), and a number (e.g., sd1A1, sd1A2, sd1B1, etc).
^{b}All gRNA target sequences are shown as the sense strand (5′-3′) without the PAM sites.
^{c}CDS = coding sequence with size indicated as the number of encoded amino acids.

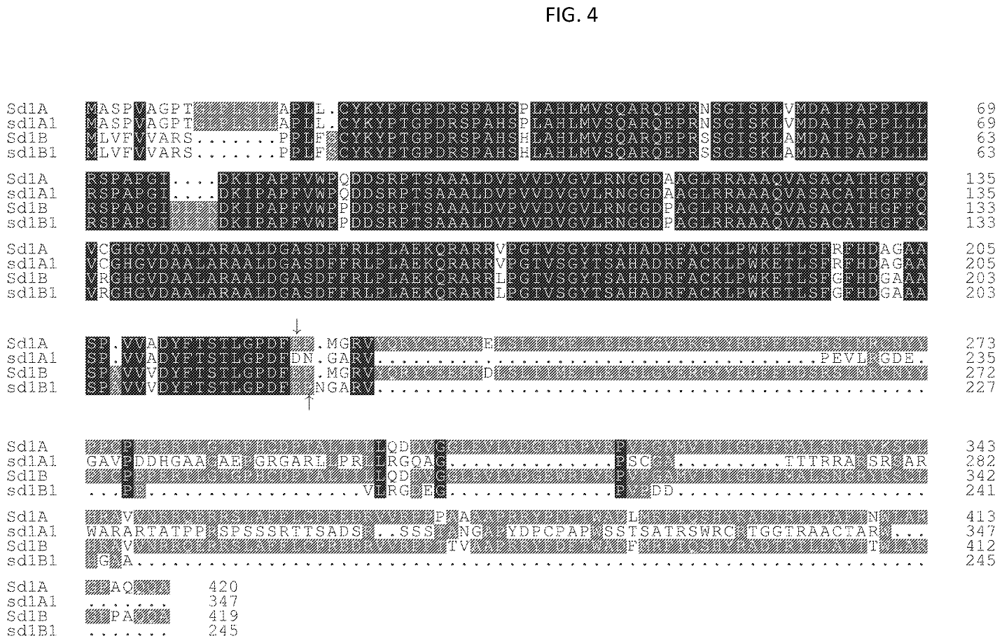

The wild-type Sd1 gene homoeologs on genome A and B encode proteins of 420 and 419 amino acids, respectively, with 93.6 percent sequence identity, that share approximately 89 percent identity with the rice GA-20 oxidase (GenBank: BAL03272.1). The sd1A1 and sd1A2 alleles, which were homozygous following two generations of selfing of the parental sd1-edited line, consisted of a dinucleotide GC deletion in the gRNA1 target site and a single adenine nucleotide insertion (+A) in the gRNA2 target site. These mutations resulted in a codon frameshift and truncation of the coding sequence to 347 amino acids (Table 3). The homozygous sd1B1 and sd1B2 alleles contained a single adenine nucleotide insertion (+A) in the gRNA1 target site that resulted in a truncated coding sequence of 245 amino acids. The nucleotide sequence corresponding to gRNA2 target site was unchanged; however, this was of no consequence as the newly created stop codon was located upstream of the gRNA2 target. Alignments of the wild-type Sd1A and Sd1B encoded sequences and the associated mutated versions are shown in FIG. 4.

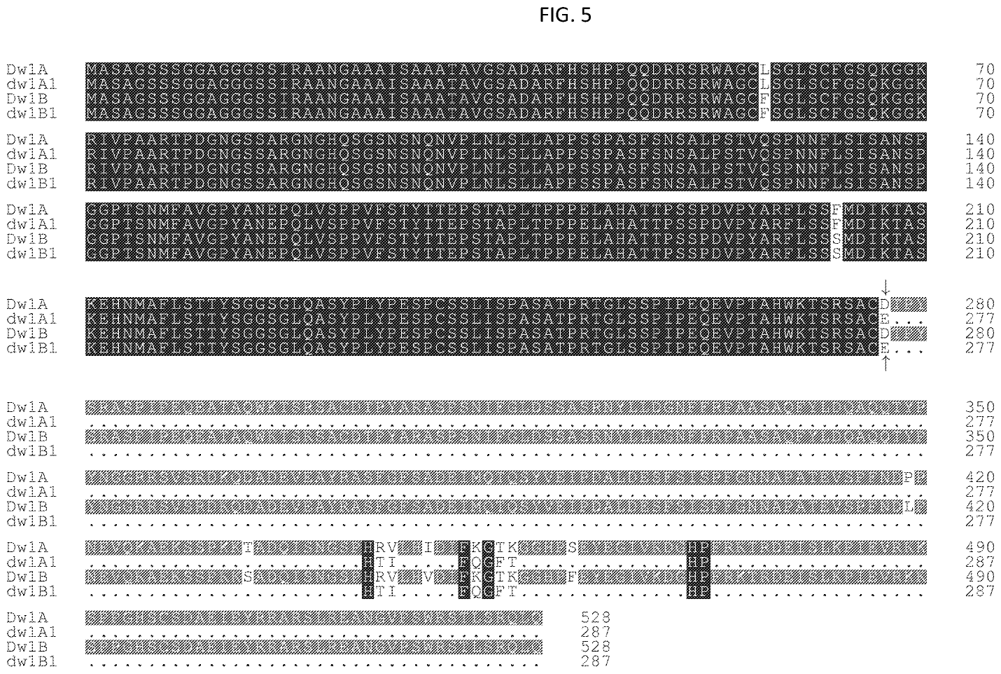

The unmodified Dw1 homoeologs encode proteins of 528 amino acids that are 99 percent sequence identical, and share 81 percent sequence identity with the homologous protein from sorghum (UniProtKB-A0A1B6P9X8). The dw1 mutation consisted of a single adenine nucleotide insertion for each homoeoallele at the same position within the gRNA target sequence (Table 3), which resulted in a truncated open reading frame (ORF) of 287 amino acids for each homoeologous gene. Alignments of the amino acid sequences encoded by the wild-type Dw1 and mutant dw1 alleles are shown in FIG. 5.

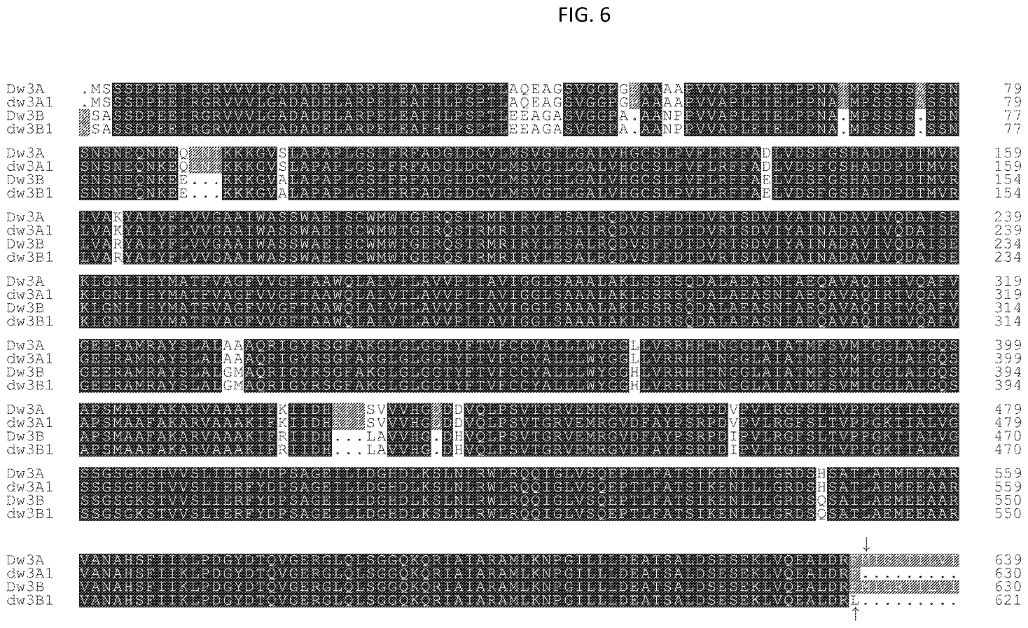

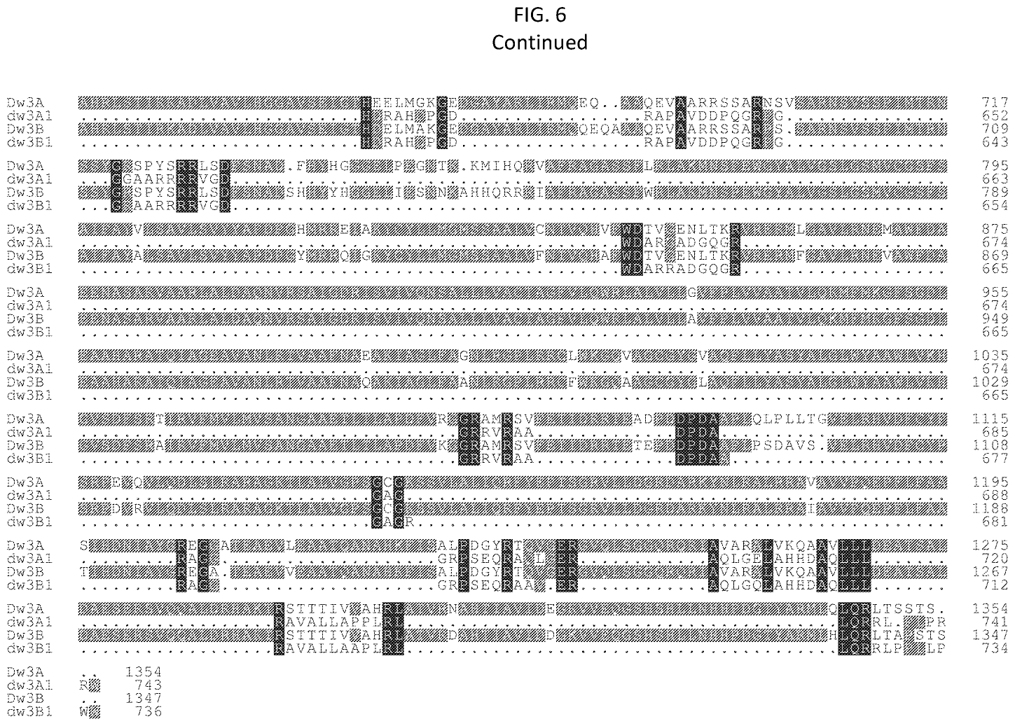

The unmodified Dw3 homoeologs on the A and B genomes encode proteins of 1354 and 1347 amino acids, respectively, that share 93 percent sequence identity. The teff DW3A and DW3B proteins are, respectively, 87 percent and 88 percent identical to the homologous protein from sorghum (UniProtKB-A0A1Z5RA91). The dw3 mutation consisted of a single thymine nucleotide insertion in the gRNA target site of the A genome (dw3A) and a corresponding single adenine nucleotide insertion in the gRNA target site of the B genome (dw3B) (Table 3). The dw3A and dw3B mutations resulted in truncated ORFs of 743 and 736 amino acids, respectively. Alignments of the wild-type Dw3A and Dw3B encoded sequences and the associated mutated versions are shown in FIG. 6.

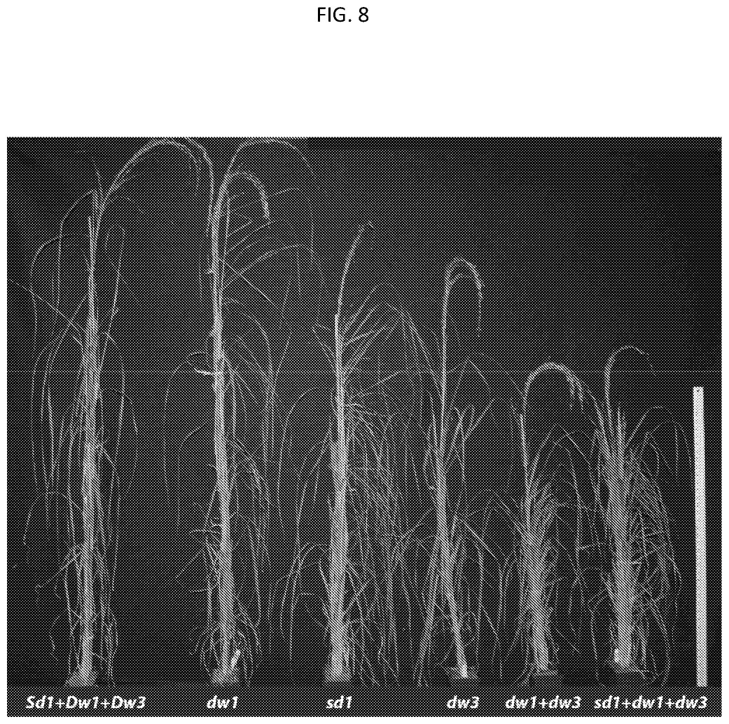

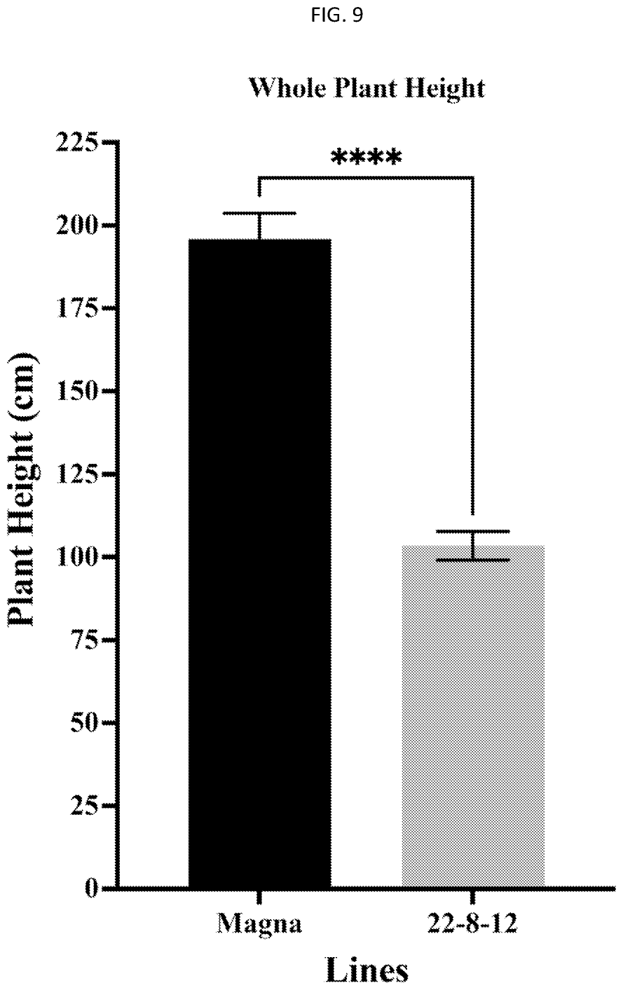

Phenotypes of the combination and subcombinations of sd1, dw1, and dw3 mutant alleles are shown in FIG. 8. As shown in FIG. 8, the effect of each mutation and each combination of mutations on height of plants cannot be predicted from the phenotype of each individual mutation. For instance, although plants containing individual mutations did not show any difference in height (dw1) or a small difference in height (dw3) when compared to the height of wt plants, the dw1 and dw3 mutations generated synergistically shorter plants in combination. A quantitative measurement of the heights of wild type plants and teff plants comprising the sd1, dw1, and dw3 mutation.

### Illustrative Aspects of the Disclosure Include

Aspect 1. A genetically modified lodging resistant Eragrostis plant, part thereof, or plant cell thereof, the plant comprising two or more nucleic acid modifications that result in a reduction in two or more hormone activities selected from auxin transport activity, gibberellic acid (GA) biosynthesis activity, and brassinosteroid (BR) signaling activity when compared to the hormone activities in a wild type plant, wherein lodging resistance negatively correlates with plant height, and wherein the two or more nucleic acid modifications result in a synergistic reduction in height of the Eragrostis plant.

Aspect 2. The genetically modified plant part thereof, or plant cell thereof, wherein the nucleic acid modification that results in a reduced GA biosynthesis activity is a nucleic acid modification in a nucleic acid sequence encoding an SD1 protein, wherein the nucleic acid modification that results in a reduced BR signaling activity is a nucleic acid modification in a nucleic acid sequence encoding a DW1 protein, and wherein the nucleic acid modification that results in a reduced auxin transport activity is a nucleic acid modification in a nucleic acid sequence encoding a DW3 protein.

Aspect 3. The genetically modified plant, part thereof, or plant cell thereof, wherein the plant comprises two or more nucleic acid modifications that result in a reduction of auxin transport activity when compared to the hormone activities in a wild type plant and

BR signaling activity when compared to the hormone activities in a wild type plant.

Aspect 4. The genetically modified plant of any one of claim 1 or claim 2, part thereof, or plant cell thereof, wherein the plant comprises three or more nucleic acid modifications that result in a reduction of auxin transport activity, GA biosynthesis activity, and BR signaling activity.

Aspect 5. The genetically modified plant of any of the preceding claims, part thereof, or plant cell thereof, wherein the genetically modified lodging resistant Eragrostis plant is Eragrostis teff.

Aspect 6. The genetically modified plant of claim 5, part thereof, or plant cell thereof, wherein the nucleic acid modification that results in a reduction in GA biosynthesis when compared to the hormone activities in a wild type plant is a nucleic acid modification in a nucleic acid sequence encoding an SD1A protein and a nucleic acid modification in a nucleic acid sequence encoding an SD1B protein.

Aspect 7. The genetically modified plant of claim 6, part thereof, or plant cell thereof, wherein the SD1A protein comprises an amino acid sequence comprising at least about 75% or more, at least about 85% or more, at least about 95% or more, or 100% sequence identity with an amino acid sequence of SEQ ID NO: 17; and the SD1B protein comprises an amino acid sequence comprising at least about 75% or more, at least about 85% or more, at least about 95% or more, or 100% sequence identity with an amino acid sequence of SEQ ID NO: 19, or a combination thereof.

Aspect 8. The genetically modified plant of claim 6, part thereof, or plant cell thereof, wherein the SD1A protein is encoded by a nucleic acid sequence comprising at least about 75% or more, at least about 85% or more, at least about 95% or more, or 100% sequence identity with a nucleic acid sequence of SEQ ID NO: 18, and the SD1B protein is encoded by a nucleic acid sequence comprising at least about 75% or more, at least about 85% or more, at least about 95% or more, or 100% sequence identity with a nucleic acid sequence of SEQ ID NO: 20.

Aspect 9. The genetically modified plant of claim 6, part thereof, or plant cell thereof, wherein the nucleic acid modification in the nucleic acid sequence encoding an SD1A protein comprises at least about 75% or more, at least about 85% or more, at least about 95% or more, or 100% sequence identity with a nucleic acid sequence of SEQ ID NO: 21, and the nucleic acid modification in the nucleic acid sequence encoding an SD1B protein comprises at least about 75% or more, at least about 85% or more, at least about 95% or more, or 100% sequence identity with a nucleic acid sequence of SEQ ID NO: 22.

Aspect 10. The genetically modified plant of claim 5, part thereof, or plant cell thereof, wherein the nucleic acid modification that results in a reduction in BR signaling activity when compared to the hormone activities in a wild type plant is a nucleic acid modification in a nucleic acid sequence encoding a DW1A protein and a nucleic acid modification in a nucleic acid sequence encoding a DW1B protein.

Aspect 11. The genetically modified plant of claim 10, part thereof, or plant cell thereof, wherein the DW1A protein comprises an amino acid sequence comprising at least about 75% or more, at least about 85% or more, at least about 95% or more, or 100% sequence identity with an amino acid sequence of SEQ ID NO: 23; and the DW1B protein comprises an amino acid sequence comprising at least about 75% or more, at least about 85% or more, at least about 95% or more, or 100% sequence identity with an amino acid sequence of SEQ ID NO: 25, or a combination thereof.

Aspect 12. The genetically modified plant of claim 11, part thereof, or plant cell thereof, wherein the DW1A protein is encoded by a nucleic acid sequence comprising at least about 75% or more, at least about 85% or more, at least about 95% or more, or 100% sequence identity with a nucleic acid sequence of SEQ ID NO: 24, and the DW1B protein is encoded by a nucleic acid sequence comprising at least about 75% or more, at least about 85% or more, at least about 95% or more, or 100% sequence identity with a nucleic acid sequence of SEQ ID NO: 26.

Aspect 13. The genetically modified plant of claim 10, part thereof, or plant cell thereof, wherein the nucleic acid modification in the nucleic acid sequence encoding the DW1A protein comprises at least about 75% or more, at least about 85% or more, at least about 95% or more, or 100% sequence identity with a nucleic acid sequence of SEQ ID NO: 27, and the nucleic acid modification in the nucleic acid sequence encoding an DW1B protein comprises at least about 75% or more, at least about 85% or more, at least about 95% or more, or 100% sequence identity with a nucleic acid sequence of SEQ ID NO: 28.

Aspect 14. The genetically modified plant of claim 5, part thereof, or plant cell thereof, wherein the nucleic acid modification that results in a reduction in auxin transport activity when compared to the hormone activities in a wild type plant is a nucleic acid modification in a nucleic acid sequence encoding an DW3A protein and a nucleic acid modification in a nucleic acid sequence encoding an DW3B protein.

Aspect 15. The genetically modified plant of claim 14, part thereof, or plant cell thereof, wherein an amino acid sequence of the DW3A protein comprises at least about 75% or more, at least about 85% or more, at least about 95% or more, or 100% sequence identity with an amino acid sequence of SEQ ID NO: 29 and an amino acid sequence of the DW3B protein comprises at least about 75% or more, at least about 85% or more, at least about 95% or more, or 100% sequence identity with an amino acid sequence of SEQ ID NO: 31, or a combination thereof.

Aspect 16. The genetically modified plant of claim 15, part thereof, or plant cell thereof, wherein the DW3A protein is encoded by a nucleic acid sequence comprising at least about 75% or more, at least about 85% or more, at least about 95% or more, or 100% sequence identity with a nucleic acid sequence of SEQ ID NO: 30, and the DW3B protein is encoded by a nucleic acid sequence comprising at least about 75% or more, at least about 85% or more, at least about 95% or more, or 100% sequence identity with a nucleic acid sequence of SEQ ID NO: 32.

Aspect 17. The genetically modified plant of claim 14, part thereof, or plant cell thereof, wherein the nucleic acid modification in the nucleic acid sequence encoding the DW3A protein comprises at least about 75% or more, at least about 85% or more, at least about 95% or more, or 100% sequence identity with a nucleic acid sequence of SEQ ID NO: 33, and the nucleic acid modification in the nucleic acid sequence encoding an DW3B protein comprises at least about 75% or more, at least about 85% or more, at least about 95% or more, or 100% sequence identity with a nucleic acid sequence of SEQ ID NO: 34.

Aspect 18. The genetically modified plant of claim 5, part thereof, or plant cell thereof, wherein the genetically modified lodging resistant Eragrostis teff plant comprises a nucleic acid modification in a nucleic acid sequence encoding a DW1A protein, a nucleic acid modification in a nucleic acid sequence encoding a DW1B protein, a nucleic acid modification in a nucleic acid sequence encoding a DW3A protein, and a nucleic acid modification in a nucleic acid sequence encoding a DW3B protein.

Aspect 19. The genetically modified plant of claim 5, part thereof, or plant cell thereof, wherein the genetically modified lodging resistant Eragrostis tef plant comprises a nucleic acid modification in a nucleic acid sequence encoding an SD1A protein, a nucleic acid modification in a nucleic acid sequence encoding an SD1B protein, a nucleic acid modification in a nucleic acid sequence encoding a DW1A protein, a nucleic acid modification in a nucleic acid sequence encoding a DW1B protein, a nucleic acid modification in a nucleic acid sequence encoding a DW3A protein, and a nucleic acid modification in a nucleic acid sequence encoding a DW3B protein.

Aspect 20. The genetically modified plant of any of the preceding claims, part thereof, or plant cell thereof, wherein the synergistic reduction in height comprises an approximate 50 percent reduction in height of the plant when compared to wild type plants, or plants comprising one of the nucleic acid modifications.

What is claimed is:

1. A genetically modified lodging resistant Eragrostis plant, part thereof, or plant cell thereof, the plant comprising two or more nucleic acid modifications that result in a reduction in two or more hormone activities selected from auxin transport activity, gibberellic acid (GA) biosynthesis activity, and brassinosteroid (BR) signaling activity, wherein lodging resistance negatively correlates with plant height, and wherein the two or more nucleic acid modifications result in a synergistic reduction in height of the Eragrostis plant.

2. The genetically modified plant of claim 1, part thereof, or plant cell thereof, wherein the nucleic acid modification that results in a reduced GA biosynthesis activity is a nucleic acid modification in a nucleic acid sequence encoding an SD1 protein, wherein the nucleic acid modification that results in a reduced BR signaling activity is a nucleic acid modification in a nucleic acid sequence encoding a DW1 protein, and wherein the nucleic acid modification that results in a reduced auxin transport activity is a nucleic acid modification in a nucleic acid sequence encoding a DW3 protein.

3. The genetically modified plant of any one of claim 1 or claim 2, part thereof, or plant cell thereof, wherein the plant comprises two or more nucleic acid modifications that result in a reduction of auxin transport activity and BR signaling activity.

4. The genetically modified plant of any one of claim 1 or claim 2, part thereof, or plant cell thereof, wherein the plant comprises three or more nucleic acid modifications that result in a reduction of auxin transport activity, GA biosynthesis activity, and BR signaling activity.

5. The genetically modified plant of any of the preceding claims, part thereof, or plant cell thereof, wherein the genetically modified lodging resistant Eragrostis plant is Eragrostis teff.

6. The genetically modified plant of claim 5, part thereof, or plant cell thereof, wherein the nucleic acid modification that results in a reduction in GA biosynthesis is a nucleic acid modification in a nucleic acid sequence encoding an SD1A protein and a nucleic acid modification in a nucleic acid sequence encoding an SD1B protein.

7. The genetically modified plant of claim 6, part thereof, or plant cell thereof, wherein the SD1A protein comprises an amino acid sequence comprising at least about 75% or more, at least about 85% or more, at least about 95% or more, or 100% sequence identity with an amino acid sequence of SEQ ID NO: 17; and the SD1B protein comprises an amino acid sequence comprising at least about 75% or more, at least about 85% or more, at least about 95% or more, or 100% sequence identity with an amino acid sequence of SEQ ID NO: 19, or a combination thereof.

8. The genetically modified plant of claim 6, part thereof, or plant cell thereof, wherein the SD1A protein is encoded by a nucleic acid sequence comprising at least about 75% or more, at least about 85% or more, at least about 95% or more, or 100% sequence identity with a nucleic acid sequence of SEQ ID NO: 18, and the SD1B protein is encoded by a nucleic acid sequence comprising at least about 75% or more, at least about 85% or more, at least about 95% or more, or 100% sequence identity with a nucleic acid sequence of SEQ ID NO: 20.

9. The genetically modified plant of claim 6, part thereof, or plant cell thereof, wherein the nucleic acid modification in the nucleic acid sequence encoding an SD1A protein comprises at least about 75% or more, at least about 85% or more, at least about 95% or more, or 100% sequence identity with a nucleic acid sequence of SEQ ID NO: 21, and the nucleic acid modification in the nucleic acid sequence encoding an SD1B protein comprises at least about 75% or more, at least about 85% or more, at least about 95% or more, or 100% sequence identity with a nucleic acid sequence of SEQ ID NO: 22.

10. The genetically modified plant of claim 5, part thereof, or plant cell thereof, wherein the nucleic acid modification that results in a reduction in BR signaling activity is a nucleic acid modification in a nucleic acid sequence encoding a DW1A protein and a nucleic acid modification in a nucleic acid sequence encoding a DW1B protein.

11. The genetically modified plant of claim 10, part thereof, or plant cell thereof, wherein the DW1A protein comprises an amino acid sequence comprising at least about 75% or more, at least about 85% or more, at least about 95% or more, or 100% sequence identity with an amino acid sequence of SEQ ID NO: 23; and the DW1B protein comprises an amino acid sequence comprising at least about 75% or more, at least about 85% or more, at least about 95% or more, or 100% sequence identity with an amino acid sequence of SEQ ID NO: 25, or a combination thereof.

12. The genetically modified plant of claim 11, part thereof, or plant cell thereof, wherein the DW1A protein is encoded by a nucleic acid sequence comprising at least about 75% or more, at least about 85% or more, at least about 95% or more, or 100% sequence identity with a nucleic acid sequence of SEQ ID NO: 24, and the DW1B protein is encoded by a nucleic acid sequence comprising at least about 75% or more, at least about 85% or more, at least about 95% or more, or 100% sequence identity with a nucleic acid sequence of SEQ ID NO: 26.

13. The genetically modified plant of claim 10, part thereof, or plant cell thereof, wherein the nucleic acid modification in the nucleic acid sequence encoding the DW1A protein comprises at least about 75% or more, at least about 85% or more, at least about 95% or more, or 100% sequence identity with a nucleic acid sequence of SEQ ID NO: 27, and the nucleic acid modification in the nucleic acid sequence encoding an DW1B protein comprises at least about 75% or more, at least about 85% or more, at least about 95% or more, or 100% sequence identity with a nucleic acid sequence of SEQ ID NO: 28.

14. The genetically modified plant of claim 5, part thereof, or plant cell thereof, wherein the nucleic acid modification that results in a reduction in auxin transport activity is a nucleic acid modification in a nucleic acid sequence encoding an DW3A protein and a nucleic acid modification in a nucleic acid sequence encoding an DW3B protein.

15. The genetically modified plant of claim 14, part thereof, or plant cell thereof, wherein an amino acid sequence of the DW3A protein comprises at least about 75% or more, at least about 85% or more, at least about 95% or more, or 100% sequence identity with an amino acid sequence of SEQ ID NO: 29 and an amino acid sequence of the DW3B protein comprises at least about 75% or more, at least about 85% or more, at least about 95% or more, or 100% sequence identity with an amino acid sequence of SEQ ID NO: 31, or a combination thereof.

16. The genetically modified plant of claim 15, part thereof, or plant cell thereof, wherein the DW3A protein is encoded by a nucleic acid sequence comprising at least about 75% or more, at least about 85% or more, at least about 95% or more, or 100% sequence identity with a nucleic acid sequence of SEQ ID NO: 30, and the DW3B protein is encoded by a nucleic acid sequence comprising at least about 75% or more, at least about 85% or more, at least about 95% or more, or 100% sequence identity with a nucleic acid sequence of SEQ ID NO: 32.

17. The genetically modified plant of claim 14, part thereof, or plant cell thereof, wherein the nucleic acid modification in the nucleic acid sequence encoding the DW3A protein comprises at least about 75% or more, at least about 85% or more, at least about 95% or more, or 100% sequence identity with a nucleic acid sequence of SEQ ID NO: 33, and the nucleic acid modification in the nucleic acid sequence encoding an DW3B protein comprises at least about 75% or more, at least about 85% or more, at least about 95% or more, or 100% sequence identity with a nucleic acid sequence of SEQ ID NO: 34.

18. The genetically modified plant of claim 5, part thereof, or plant cell thereof, wherein the genetically modified lodging resistant Eragrostis teff plant comprises a nucleic acid modification in a nucleic acid sequence encoding a DW1A protein, a nucleic acid modification in a nucleic acid sequence encoding a DW1B protein, a nucleic acid modification in a nucleic acid sequence encoding a DW3A protein, and a nucleic acid modification in a nucleic acid sequence encoding a DW3B protein.

19. The genetically modified plant of claim 5, part thereof, or plant cell thereof, wherein the genetically modified lodging resistant Eragrostis tef plant comprises a nucleic acid modification in a nucleic acid sequence encoding an SD1A protein, a nucleic acid modification in a nucleic acid sequence encoding an SD1B protein, a nucleic acid modification in a nucleic acid sequence encoding a DW1A protein, a nucleic acid modification in a nucleic acid sequence encoding a DW1B protein, a nucleic acid modification in a nucleic acid sequence encoding a DW3A protein, and a nucleic acid modification in a nucleic acid sequence encoding a DW3B protein.

20. The genetically modified plant of any of the preceding claims, part thereof, or plant cell thereof, wherein the synergistic reduction in height comprises an approximate 50 percent reduction in height of the plant.

21. An expression construct for introducing a nucleic acid modification in a nucleic acid sequence in an Eragrostis plant, part thereof, or plant cell thereof, wherein the expression construct comprises a promoter operably linked to a polynucleotide sequence encoding a programmable nucleotide modification system targeted to a polynucleotide in a nucleic acid sequence encoding an SD1 protein, a DW1 protein, or a DW3 protein, wherein expression of the programmable nucleotide modification system introduces a nucleic acid modification into the nucleic acid sequence, and wherein the nucleic acid modification in the nucleic acid sequence encoding an SD1 protein results in a reduction in GA biosynthesis activity, the nucleic acid modification in the nucleic acid sequence encoding a DW1 protein results in a reduction in BR signaling activity, and the nucleic acid modification in the nucleic acid sequence encoding a DW3 protein results in a reduction in auxin transport activity.

22. The expression construct of claim 21, wherein the programmable nucleotide modification system is selected from an RNA-guided clustered regularly interspersed short palindromic repeats (CRISPR)/CRISPR-associated9 (Cas) (CRISPR/Cas9) nuclease system, a CRISPR/Cpf1 nuclease system, a zinc finger nuclease (ZFN), a transcription activator-like effector nuclease (TALEN), a meganuclease, ribozyme, and a programmable DNA binding domain linked to a nuclease domain

23. The expression construct of claim 21, wherein the programmable nucleic acid modification system is a CRISPR/Cas system comprising a Cas9 nuclease and a guide RNAs (gRNA) comprising a sequence complementary to a target sequence within the nucleic acid sequence.

24. The expression construct of claim 23, wherein the Eragrostis plant, part thereof, or plant cell thereof is Eragrostis tef.

25. The expression construct of claim 24, wherein the nucleic acid sequence encodes an SD1A protein or an SD1B protein and the gRNA comprises a nucleic acid sequence of SEQ ID NO: 35 (Sd1 gRNA1), SEQ ID NO: 36 (Sd1 gRNA2), or a combination thereof.

26. The expression construct of claim 24, wherein the nucleic acid sequence encodes an DW1A protein or an DW1B protein and the gRNA comprises a nucleic acid sequence of SEQ ID NO: 37 (Dw1 gRNA).

27. The expression construct of claim 26, wherein the nucleic acid sequence encodes an DW3A protein or an DW3B protein and the gRNA comprises a nucleic acid sequence of SEQ ID NO: 38 (Dw3 gRNA).

28. The expression construct of claim 21, further comprising a nucleic acid delivery vector comprising the nucleic acid expression construct for delivering the nucleic acid expression construct to a target cell.

29. A plant, part thereof, or plant cell thereof comprising one or more expression constructs of claims 21-28, one or more vector of claim 29, or any combination thereof.

30. A method of producing a lodging resistant Eragrostis plant, part thereof, or plant cell thereof, the method comprising: introducing into the plant two or more nucleic acid modifications that result in a reduction in two or more plant hormone activities selected from auxin transport activity, gibberellic acid (GA) biosynthesis activity, and brassinosteroid (BR) signaling activity.

31. The method of claim 31, wherein the nucleic acid modifications are introduced using a programmable nucleotide modification system.

32. The method of claim 32, wherein the programmable nucleotide modification system is selected from an RNA-guided clustered regularly interspersed short palindromic repeats (CRISPR)/CRISPR-associated9 (Cas) (CRISPR/Cas9) nuclease system, a CRISPR/Cpf1 nuclease system, a zinc finger nuclease (ZFN), a transcription activator-like effector nuclease (TALEN), a meganuclease, ribozyme, and a programmable DNA binding domain linked to a nuclease domain.

33. A method of improving the yield of an Eragrostis plant, part thereof, or plant cell thereof, the method comprising using a method of claims 31-33 to generate a genetically modified lodging resistant Eragrostis plant of claim 1-20.

34. A kit for improving the yield of an Eragrostis plant, part thereof, or plant cell thereof, the kit comprising:
a. one or more genetically modified lodging resistant Eragrostis plant, part thereof, or plant cell thereof of claims 1-20;
b. one or more expression constructs of claims 21-28 for introducing a nucleic acid modification in a nucleic acid sequence in an Eragrostis plant;
c. one or more genetically modified lodging resistant Eragrostis plant, part thereof, or plant cell thereof of claim 29 one or more expression constructs of claims 21-28; or
d. any combination of (a)-(c).


SEQUENCE LISTING (40 sequences)

SEQ ID NO: 1 - DNA, 20 bp, Eragrostis tef
  source 1..20: mol_type = other DNA
ggcccggacttcgagccaat

SEQ ID NO: 2 - DNA, 18 bp, synthetic construct
  source 1..18: mol_type = other DNA
ggcccggacttcgacaat

SEQ ID NO: 3 - DNA, 20 bp, Eragrostis tef
  source 1..20: mol_type = other DNA
gcgacttcttcgaggacagc

SEQ ID NO: 4 - DNA, 21 bp, synthetic construct
  source 1..21: mol_type = other DNA
gcgacttcttcgaggacaagc

SEQ ID NO: 5 - DNA, 20 bp, Eragrostis tef
  source 1..20: mol_type = other DNA
ggcccggacttcgagccaat

SEQ ID NO: 6 - DNA, 21 bp, synthetic construct
  source 1..21: mol_type = other DNA
ggcccggacttcgagccaaat

SEQ ID NO: 7 - DNA, 20 bp, Eragrostis tef
  source 1..20: mol_type = other DNA
gcgacttcttcgaggacagc

SEQ ID NO: 8 - DNA, 20 bp, synthetic construct
  source 1..20: mol_type = other DNA
gcgacttcttcgaggacagc

SEQ ID NO: 9 - DNA, 20 bp, Eragrostis tef
  source 1..20: mol_type = other DNA
gtgacacaccatattccagg

SEQ ID NO: 10 - DNA, 21 bp, synthetic construct
  source 1..21: mol_type = other DNA
gtgaacacaccatattccagg

SEQ ID NO: 11 - DNA, 20 bp, Eragrostis tef
  source 1..20: mol_type = other DNA
gtgacacaccatattccagg

SEQ ID NO: 12 - DNA, 21 bp, synthetic construct
  source 1..21: mol_type = other DNA
gtgaacacaccatattccagg

SEQ ID NO: 13 - DNA, 20 bp, Eragrostis tef
  source 1..20: mol_type = other DNA
gttcatgatcgggcgcacga

SEQ ID NO: 14 - DNA, 21 bp, synthetic construct
  source 1..21: mol_type = other DNA
gtttcatgatcgggcgcacga

SEQ ID NO: 15 - DNA, 20 bp, Eragrostis tef
  source 1..20: mol_type = other DNA
gttcatgatcgggcgcacga

SEQ ID NO: 16 - DNA, 21 bp, synthetic construct
  source 1..21: mol_type = other DNA
gttacatgatcgggcgcacga

SEQ ID NO: 17 - protein, 420 aa, Eragrostis tef
MASPVAGPTGLPLSLLAPLLCYKYPTGPDRSPAHSPLAHLMVSQARQEPRNSGISKLVMDAIPAPPLLLRSPAPGIDKIPAPFVWPQDDSRPTSAAALDVPVVDVGVLRNGGDAAGLRRAAAQVASACATHGFFQVCGHGVDAALARAALDGASDFFRLPLAEKQRARRVPGTVSGYTSAHADRFACKLPWKETLSFRFHDAGAASPVVADYFTSTLGPDFEPMGRVYQRYCEEMKELSLTIMELLELSLGVERGYYRDFFEDSRSIMRCNYYPPCPEPERTLGTGPHCDPTALTILLQDDVGGLEVLVDGEWRPVRPVPGAMVINIGDTFMALSNGRYKSCLHRAVVNRRQERRSLAFFLCPREDRVVRPPPAAAAPRRYPDFTWADLMRFTQSHYRADTRTLDAFTNWLARGPAQQQA

SEQ ID NO: 18 - DNA, 2364 bp, Eragrostis tef
  source 1..2364: mol_type = other DNA
atggcgtcccctgtggcaggccccacagggttgcctttgtcgttgcttgcccctctcctctgttacaaataccccaccggtccggacaggtcccctgcacactcacccctcgcacatctcatggtgtcccaagcacggcaagagcctcgcaacagcggcatctcgaagctcgtcatggacgccatcccggcccctcctctcctgctccgctctccagctcccggcattgacaagatcccccaagatcccctgtggccgcaggacgactcgcgcccgacgtcggcggcggcgctggacgtccccgtggtggacgtgggcgtgctccgcaatggcggcgacgccgccgggctgcggcgcgcggcggcgcaggtggcgtcggcgtgcgcgacgcacggcttcttccaggtgtgcgggcacggcgtggacgcggccctggcgcgcgccgcgctggacggcgcatccgacttcttccggctgccgctggccgagaagcagcgcgcccggcgcgtgccaggcaccgtgtccgggtacacgagcgcgcacgccgaccggttcgcgtgcaagctgccctggaaggagacgctgtccttccgcttccacgacgccggcgccgcgtcgcccgtggtcgccgactacttcaccagcaccctcggcccggacttcgagccaatggggtacgtaaccaaccacgttgactggtttaattaattactgcatatatagtatccatcatctccatggatatatatatatatatatatatatatatatatatatatatatatatatatatatatcttggcattgcatcattggacacgtacacacggacggctgaaaaaactatatggcaggcgcgtgtaccagaggtactgcgaggagatgaaggagctgtccctgacgatcatggagctgctggagctgagcctgggcgtggagcgcggctactaccgcgacttcttcgaggacagccggtccatcatgcggtgcaactactacccgccgtgcccggagccggagcgcacgctgggcacgggcccgcactgcgaccccaccgcgctcaccatcctcctccaggacgacgtcggcggactcgaggtcctcgtcgacggcgaatggcgccccgtacgacccgtgcccggcgccatggtcatcaacatcggcgacacgttcatggtatggtattgtattactctgctgctcttggctgcctgcttgcttgtcctgcccctgtttctcctcctaatggaagtgattggacagtcgcagattacacacactcacacgtagctgcctaggcgtgtcatgttgcgcaatgtagacatgcgttcgagttggttccatctcgatcggtgggctagctcggtagtacaggtctatcaatcggtggtggcgaacggcgagccgccttgatgatttcgccaaagaaaaaaaagaaagaaagagagaggttgcgatgcagtaggaggacaaagagagagagagagagagagagagagagagaagaggttggcttccgccgttggcggcttgcgagttgtagaggagtgggggaggcccggaggaggagggggaggatacgatgccgcgagcggtggcgctttcgccgggtggacccaagcccaggtgcgcgcgctcgtcgtcttccctgttttggggccgtccgggtcggtggggccatctcatctcatcccgcggcgtgactgactgtgcgctgcacgggccgccgcgctggatcatacatgggcttggcgttaccactggcagcttctacttggctgtcgctcgtcggcttctagccgtttccacggcgtttgtcgcggttttccggtgcacgcggcgcccgcctccagctcggccgtgcgggtggcacgacaccatcaccccgtcacccgactcacccgtgcgtgcggcttggctccgccacgcgggccgaaaacaaaagggttcctgtgccttgacaggtcgtcacgtgttgtgttgtgcaaaagaactccggattgagctaattgttgtttcgctcactgttgattgcgccgccgatcgccggcagcatctctgacttcagcaactgactcctctgctggttgaacgcaggcgctgtcgaacgggcggtacaagagctgcctgcaccgcgcggtggtgaaccggcggcaggagcggcggtcgctggccttcttcctgtgcccgcgcgaggaccgcgtcgtgcgccccccgccggccgccgccgcgccgcgccgctacccggacttcacgtgggccgacctcatgcgcttcacgcagagccactaccgcgccgacacacgcaccctcgacgcattcaccaactggctcgcccgcggcccggcccagcagcaggcgtag

SEQ ID NO: 19 - protein, 418 aa, Eragrostis tef
MLVFVVARSPPLFSCYKYPTGPDRSPAHSHLAHLMVSQARQEPRSSGIKLAMDAIPAPPLLLRSPAPGIDLPKDKIPAPFVWPPDDSRPTSAAALDVPVVDVGVLRNGGDPAGLRRAAAQVASACATHGFFQVRGHGVDAALARAALDGASDFFRLPLAEKQRARRLPGTVSGYTSAHADRFACKLPWKETLSFGFHDGAAASPAVVVDYFTSTLGPDFEPMGRVYQRYCEEMKDLSLTIMELLELSLGVERGYYRDFFEDSRSIMRCNYYPPCPEPERTLGTGPHCDPTALTILLQDDVGGLEVLVDGEWRPVRPVPGAMVINIGDTFMALSNGRYKSCLHRAVVNRRQERRSLAFFLCPREDRVVRPPTTVAAPRRYPDFTWADFMRFTQSHYRADTRTLDAFTTWLARGPPAQQA

SEQ ID NO: 20 - DNA, 2273 bp, Eragrostis tef
  source 1..2273: mol_type = other DNA
atgcttgtgtttgtcgttgctcgctcccctcccctgttctcctgttacaaataccccaccggcccggacaggtcccctgcacactcacacctcgcacatctcatggtgtcccaagcacggcaagagcctcgcagcagcggcatctccaagctcgccatggacgccatcccggccccgcctctcctgctccgctccccagctcccggcattgacctccccaaagacaagatccccgcgccgttcgtgtggccgccggacgactcgcggccgacgtcggcggcggcgctggacgtccccgtggtggacgtgggcgtgctgcgcaatggcggcgaccccgccgggctgcggcgcgcggcggcgcaggtggcgtcggcgtgcgcgacgcacggcttcttccaggtgcgcgggcacggcgtggacgccgccctggcgcgcgcggcgctggacggcgccagcgacttcttccggctgccgctcgccgagaagcagcgcgcgcggcgactccccggcaccgtgtccgggtacacgagcgcccacgccgaccggttcgcgtgcaagctcccgtggaaggagacgctgtccttcggcttccacgacggcgccgctgcgtcgccggccgtcgtcgtcgactacttcaccagcaccctcggcccggacttcgagccaatggggtaacatataaacactactggcgggcttgctgtggatatagtatcttgtcgccattgcattcgacaccgacactagcttgggacgactggaaccatccatcatgtactacttatatggcaggcgcgtgtaccagaggtactgcgaggagatgaaggacctgtccctgacgatcatggagctgcttgagctgagcctgggcgtggagcgcggctactaccgcgacttcttcgaggacagccggtccatcatgcggtgcaactactacccgccgtgcccggagccggagcgcacgctgggcacgggcccgcactgcgaccccacggcgctcaccatcctgctccaggacgacgtcggcggactcgaggtcctcgtcgacggggaatggcgccccgtccgccccgtgcccggagccatggtcatcaacatcggcgacacgttcatggtacaattcaatttactctgttctttggctggctgctagcttgtcctgccctgttcccgatgtcctcctagctaatggaactaattggacagcgctcagattacacacacacgtaggcgtgtcatgttgcgcaatgtagacatgcgttcgagttggttccatctcgatccgtgggctagctcggtagtaggtctaccggtggtggtgaacggtgagccgccttgatggtttcgcgaaagaagaaaaaagagagagagagagagagagagagaggtagcgatgcagtaggaggacaaagagagagagagaagaggttggcttgtgccgttggcggcttgcgagttgtagaggagtgggggaggaccggaggaggagggggatacgatgccgcgagcggtggcgctttcgcctggtggccccaagcccaggtgcgcgcgcgctcgttgtcttccctgttttgggcccgtccggggccgtctcatccgcggcgtgactgtgcgccgcacgggccgccgcgctggagcacaggatcgtcagcttggcgttacccgctggtatgctctgcgtaattctacttggctgtcgctcgccgggttctagccgtttccgcggcgtttggcgcggtttcccggtgcacgcggcgcccgcgcgggggctccagctcggtgcgggtggcactacgctagcgtatagtacgcgcgccgcaacaaaagggttcctgtgccttgacaagccttcactcacgtctagtgtttgcacaaaaggggggggtttccggattgagctgattgttgtttcgttccagtgttcactgttgatctgcatctctccctgactgactgactgactgactgactgacttctctactggttgtttgcacactggcaacaggcgctgtccaacgggcggtacaagagctgcctgcaccgcgcggtggtgaaccggcggcaggagcggcggtcgctggccttcttcctgtgcccgcgcgaggaccgcgtcgtgcgcccgccgaccaccgtcgccgcgccgcgccgctacccggacttcacgtgggccgacttcatgcgcttcacgcagagccactaccgcgccgacacacgcaccctcgacgccttcaccacctggctcgcccgcggcccgccggcccagcaggcgtag

SEQ ID NO: 21 - DNA, 2363 bp, synthetic construct
  source 1..2363: mol_type = other DNA
atggcgtcccctgtggcaggccccacagggttgcctttgtcgttgcttgcccctctcctctgttacaaataccccaccggtccggacaggtcccctgcacactcacccctcgcacatctcatggtgtcccaagcacggcaagagcctcgcaacagcggcatctcgaagctcgtcatggacgccatcccggcccctcctctcctgctccgctctccagctcccggcattgacaagatcccccaagatcccctgtggccgcaggacgactcgcgcccgacgtcggcggcggcgctggacgtccccgtggtggacgtgggcgtgctccgcaatggcggcgacgccgccgggctgcggcgcgcggcggcgcaggtggcgtcggcgtgcgcgacgcacggcttcttccaggtgtgcgggcacggcgtggacgcggccctggcgcgcgccgcgctggacggcgcatccgacttcttccggctgccgctggccgagaagcagcgcgcccggcgcgtgccaggcaccgtgtccgggtacacgagcgcgcacgccgaccggttcgcgtgcaagctgccctggaaggagacgctgtccttccgcttccacgacgccggcgccgcgtcgcccgtggtcgccgactacttcaccagcaccctcggcccggacttcgacaatggggtacgtaaccaaccacgttgactggtttaattaattactgcatatatagtatccatcatctccatggatatatatatatatatatatatatatatatatatatatatatatatatatatatatcttggcattgcatcattggacacgtacacacggacggctgaaaaaactatatggcaggcgcgtgtaccagaggtactgcgaggagatgaaggagctgtccctgacgatcatggagctgctggagctgagcctgggcgtggagcgcggctactaccgcgacttcttcgaggacaagccggtccatcatgcggtgcaactactacccgccgtgcccggagccggagcgcacgctgggcacgggcccgcactgcgaccccaccgcgctcaccatcctcctccaggacgacgtcggcggactcgaggtcctcgtcgacggcgaatggcgccccgtacgacccgtgcccggcgccatggtcatcaacatcggcgacacgttcatggtatggtattgtattactctgctgctcttggctgcctgcttgcttgtcctgcccctgtttctcctcctaatggaagtgattggacagtcgcagattacacacactcacacgtagctgcctaggcgtgtcatgttgcgcaatgtagacatgcgttcgagttggttccatctcgatcggtgggctagctcggtagtacaggtctatcaatcggtggtggcgaacggcgagccgccttgatgatttcgccaaagaaaaaaaagaaagaaagagagaggttgcgatgcagtaggaggacaaagagagagagagagagagagagagagagagaagaggttggcttccgccgttggcggcttgcgagttgtagaggagtgggggaggcccggaggaggagggggaggatacgatgccgcgagcggtggcgctttcgccgggtggacccaagcccaggtgcgcgcgctcgtcgtcttccctgttttggggccgtccgggtcggtggggccatctcatctcatcccgcggcgtgactgactgtgcgctgcacgggccgccgcgctggatcatacatgggcttggcgttaccactggcagcttctacttggctgtcgctcgtcggcttctagccgtttccacggcgtttgtcgcggttttccggtgcacgcggcgcccgcctccagctcggccgtgcgggtggcacgacaccatcaccccgtcacccgactcacccgtgcgtgcggcttggctccgccacgcgggccgaaaacaaaagggttcctgtgccttgacaggtcgtcacgtgttgtgttgtgcaaaagaactccggattgagctaattgttgtttcgctcactgttgattgcgccgccgatcgccggcagcatctctgacttcagcaactgactcctctgctggttgaacgcaggcgctgtcgaacgggcggtacaagagctgcctgcaccgcgcggtggtgaaccggcggcaggagcggcggtcgctggccttcttcctgtgcccgcgcgaggaccgcgtcgtgcgccccccgccggccgccgccgcgccgcgccgctacccggacttcacgtgggccgacctcatgcgcttcacgcagagccactaccgcgccgacacacgcaccctcgacgcattcaccaactggctcgcccgcggcccggcccagcagcaggcgtag

SEQ ID NO: 22 - DNA, 2274 bp, synthetic construct
  source 1..2274: mol_type = other DNA
atgcttgtgtttgtcgttgctcgctcccctcccctgttctcctgttacaaataccccaccggcccggacaggtcccctgcacactcacacctcgcacatctcatggtgtcccaagcacggcaagagcctcgcagcagcggcatctccaagctcgccatggacgccatcccggccccgcctctcctgctccgctccccagctcccggcattgacctccccaaagacaagatccccgcgccgttcgtgtggccgccggacgactcgcggccgacgtcggcggcggcgctggacgtccccgtggtggacgtgggcgtgctgcgcaatggcggcgaccccgccgggctgcggcgcgcggcggcgcaggtggcgtcggcgtgcgcgacgcacggcttcttccaggtgcgcgggcacggcgtggacgccgccctggcgcgcgcggcgctggacggcgccagcgacttcttccggctgccgctcgccgagaagcagcgcgcgcggcgactccccggcaccgtgtccgggtacacgagcgcccacgccgaccggttcgcgtgcaagctcccgtggaaggagacgctgtccttcggcttccacgacggcgccgctgcgtcgccggccgtcgtcgtcgactacttcaccagcaccctcggcccggacttcgagccaaatggggtaacatataaacactactggcgggcttgctgtggatatagtatcttgtcgccattgcattcgacaccgacactagcttgggacgactggaaccatccatcatgtactacttatatggcaggcgcgtgtaccagaggtactgcgaggagatgaaggacctgtccctgacgatcatggagctgcttgagctgagcctgggcgtggagcgcggctactaccgcgacttcttcgaggacagccggtccatcatgcggtgcaactactacccgccgtgcccggagccggagcgcacgctgggcacgggcccgcactgcgaccccacggcgctcaccatcctgctccaggacgacgtcggcggactcgaggtcctcgtcgacggggaatggcgccccgtccgccccgtgcccggagccatggtcatcaacatcggcgacacgttcatggtacaattcaatttactctgttctttggctggctgctagcttgtcctgccctgttcccgatgtcctcctagctaatggaactaattggacagcgctcagattacacacacacgtaggcgtgtcatgttgcgcaatgtagacatgcgttcgagttggttccatctcgatccgtgggctagctcggtagtaggtctaccggtggtggtgaacggtgagccgccttgatggtttcgcgaaagaagaaaaaagagagagagagagagagagagagaggtagcgatgcagtaggaggacaaagagagagagagaagaggttggcttgtgccgttggcggcttgcgagttgtagaggagtgggggaggaccggaggaggagggggatacgatgccgcgagcggtggcgctttcgcctggtggccccaagcccaggtgcgcgcgcgctcgttgtcttccctgttttgggcccgtccggggccgtctcatccgcggcgtgactgtgcgccgcacgggccgccgcgctggagcacaggatcgtcagcttggcgttacccgctggtatgctctgcgtaattctacttggctgtcgctcgccgggttctagccgtttccgcggcgtttggcgcggtttcccggtgcacgcggcgcccgcgcgggggctccagctcggtgcgggtggcactacgctagcgtatagtacgcgcgccgcaacaaaagggttcctgtgccttgacaagccttcactcacgtctagtgtttgcacaaaaggggggggtttccggattgagctgattgttgtttcgttccagtgttcactgttgatctgcatctctccctgactgactgactgactgactgactgacttctctactggttgtttgcacactggcaacaggcgctgtccaacgggcggtacaagagctgcctgcaccgcgcggtggtgaaccggcggcaggagcggcggtcgctggccttcttcctgtgcccgcgcgaggaccgcgtcgtgcgcccgccgaccaccgtcgccgcgccgcgccgctacccggacttcacgtgggccgacttcatgcgcttcacgcagagccactaccgcgccgacacacgcaccctcgacgccttcaccacctggctcgcccgcggcccgccggcccagcaggcgtag

SEQ ID NO: 23 - protein, 528 aa, Eragrostis tef
MASAGSSSGGAGGGSSIRAANGAAAISAAATAVGSADARFHSHPPQQDRRSRWAGCLSGLSCFGSQKGGKRIVPAARTPDGNGSSARGNGHQSGSNSNQNVPLNLSLLAPPSSPASFSNSALPSTVQSPNNFLSISANSPGGPTSNMFAVGPYANEPQLVSPPVFSTYTTEPSTAPLTPPPELAHATTPSSPDVPYARFLSSFMDIKTASKEHNMAFLSTTYSGGSGLQASYPLYPESPCSSLISPASATPRTGLSSPIPEQEVPTAHWKTSRSACDTPYSRASPIPEQEATAQWKTSRSACDTPYARASPSNIFGLDSSASRNYLLDGNFFRPAASAQFYLDQAQQTYPYNGGRRSVSRDKQDADEVEAYRASFGFSADEIMQTQSYVEIPDALDESFSISPFGNNAPATEVSPFNDPPNEVQKAEKSSPKKTADQISNGSPHRVLHIDIFKGTKGGHPSEYEGIVKDGHPFRKTRDEISLKPIEVRKKSPPGHSCSDAEIEYRRARSLREANGVPSWRSTLSRQLQ

SEQ ID NO: 24 - DNA, 2501 bp, Eragrostis tef
  source 1..2501: mol_type = other DNA
atggcttcagctgggagcagcagtggaggtgcaggtggaggcagcagcatcagggcggcgaatggcgcagctgccatcagtgcggccgcgacggcagtaggctcagccgacgccagattccactcccacccaccacagcaggacagggtatatacactacacgacttcttgttccatggcgcatgcaattgctcatgctttagctgcagcttttacccttcttgctttagatgtgtggatgagtaccagcaatttctagtaggccttgacgacctagattgtcacaggctttgcgtacttagtacgaatggtactgcccagtggtggttgcagcttgcaaccccggcatctttacccccaataattaggcttcagtactgtctgcttgtttgcttctagtactgaatagtttgttcagctgaattttccgttttgctgttgatatttgattattacgcgatttgcactccctctatgtttttccccccggatcctagatgactgtactcttaccataacactcgtccagagacctatatgttgtgtaactgttgatttgaccttgcatacacttccacacgtcgtgtcagttgatgtcatgagcaaactaactatgctgggttgattccttcttgcatgatggatctttttccaaatgaattttcagattcctgtaaaatgcttggtggtgtagctattaatgtcaacacatttcttatattcttaagtaaaattggtttagattaatactccaaggaaatttcgttgttgaacaactagtcatacattttttcccctaccaacagctatgtacttgatactaatatgtgcatctctgatctaaacagcggagtagatgggctggctgcttgtcgggtctttcatgtttcggatctcagaagggcgggaagaggattgttcctgccgcacgtactcctgatgggaatggatcaagtgctcgtggaaatggtcatcagtccggttctaattcaaaccaaaatgtgcctttaaatctgtctcttctggctccaccatcctcaccggcatccttctcaaattctgcacttccttcaactgttcaatcgcctaataactttctgtcaatctcagcaaattctcctggtggtccaacatctaatatgtttgctgttgggccatatgctaatgaacctcagcttgtctcgcctcctgtcttctcaacttacacaactgagccttccacagcaccattgacccctccacctgaactagctcatgcaaccactccctcatctccagatgttccatacgctcggtttctttcttcttttatggatatcaaaactgctagtaaggagcataacatggctttcttatcaacaacatattctggtggttcaggactccaggcatcataccctctttaccctgagagcccttgtagcagcctcatatcacctgcttctgctactccgaggactggtctttcctcaccaatacctgaacaagaggtccctactgcccattggaagacttctaggtctgcctgtgacacaccatattccagggcttcacccatccctgagcaagaagccactgcgcagtggaagacttctagatcggcatgtgatacgccttatgccagggcttcaccatcaaacatctttgggctggactcatctgcttctagaaactatttgttagatggcaacttcttccggccagctgcttctgctcaattctacctggaccaggctcagcagacatatccatataatggtgggaggcgtagtgtctcacgggacaagcaagatgcagatgaagttgaagcttacagagcttcgtttggctttagtgcagatgaaatcatgcaaactcaatcttatgtggagataccagatgcacttgatgaatcattcagtatatcgccatttggaaataatgctcctgctactgaggtgtccccatttaatgatccacccaacgaggttcagaaggctgagaagtcaagtccaaagaaaacggcagatcagatttccaatggctctccacatagagtgctgcacattgacatattcaagggtaaatgtccatactttttgcaccaaccattcagttgtaatctaggcatagtagaattactcttgtttatccttgctatctactgcacctaacaaagctttggtaactttcaatgctaataacctgaactctgggccatttgtcatcagcacttattggtattgtgtttctatttatgtaaatgcttattatgcttatgtgctcgcatttcaggaacaaaaggaggacatccgtctgagtacgagggtattgtgaaagatggccatcctttcagaaagacaagggacgaaatatctctaaaacccatagaagtaaggaagaaatctccacctgggcattcatgctcagatgctgaaattgagtacagaagggcaaggagtctgagggaagccaatggtgtcccctcgtggcgcagcacgttgtcaagacagctgcagtga

SEQ ID NO: 25 - protein, 528 aa, Eragrostis tef
MASAGSSSGGAGGGSSIRAANGAAAISAAATAVGSADARFHSHPPQQDRRSRWAGCFSGLSCFGSQKGGKRIVPAARTPDGNGSSARGNGHQSGSNSNQNVPLNLSLLAPPSSPASFSNSALPSTVQSPNNFLSISANSPGGPTSNMFAVGPYANEPQLVSPPVFSTYTTEPSTAPLTPPPELAHATTPSSPDVPYARFLSSSMDIKTASKEHNMAFLSTTYSGGSGLQASYPLYPESPCSSLISPASATPRTGLSSPIPEQEVPTAHWKTSRSACDTPYSRASPIPEQEATAQWKTSRSACDTPYARASPSNIFGLDSSASRNYLLDGNFFRPAASAQFYLDQAQQTYPYNGGRRSVSRDKQDADEVEAYRASFGFSADEIMQTQSYVEIPDALDESFSISPFGNNAPATEVSPFNDLPNEVQKAEKSSPKKSADQISNGSPHRVLHVDIFKGTKGGHPFEYEGIVKDGHPFRKTRDEISLKPIEVRKKSPPGHSCSDAEIEYRRARSLREANGVPSWRSTLSRQLQ

SEQ ID NO: 26 - DNA, 2561 bp, Eragrostis tef
  source 1..2561: mol_type = other DNA
atggcttcagctgggagcagcagtggaggtgcaggtggaggcagcagcatcagggcggcgaatggcgcagctgctatcagtgcggccgcgacggcagtaggctcagccgacgccagattccactcccacccaccacaacaggacagggtatatacactacacgacttcttgttccatggcgcatgcaattgctcatgctttggctgcagcttttacccttcttgctttagatgtatggatgagtaccagcaatttccagtaggccttgacgacctagattgtcacaggctttgcgtacttagtacgaatggtactgcccagtggtggttgcagcttgcaacccccggcatctttacccccaataattaggcttcagtactgtctgcttgtttgcttctactgaataatttgttcggctgaatttttcgttttgctgtcgatattgattgcacgccatttgcactccctctatgtactcttaccagatgactgtttgtactcttaccataacactcctccagagatcctagatgctgtataactgttgatttgaccttggatacacttccacacgttgtgtcagttgatgtcatgagcaaactaactatgctgggctgaaatcttcttgcatgataaatctttttccatatgaaaattttcagatggctgtaaaattcttggtggtgtagctattattgtcaacaaatttcttatattctttagtaaacttgttttttatgcttacccgttatagtaagtttggcccagtttcccttcccatgattttaatgaataaatcactttttctgtttgtagcgagtatagattaatactctgaggaaatttcgttgttgaacatttagtcatacatttcctccctaccaacagctatgtacttgatactaatatgtgcatctctgatctaaacagcggagtagatgggctggctgcttttcgggcctttcatgtttcggatctcagaagggcgggaagaggattgttccggccgcacgtactcctgatgggaatggatcaagtgctcgtggaaatggtcatcagtctggttctaattcaaaccaaaatgtgcctttaaatctgtcgcttctggctccaccatcgtcaccggcatccttctcaaattctgcacttccttcaactgttcagtcgccaaataactttctgtcaatctcagcaaattctcctggtggtccgacatctaatatgtttgctgttgggccatatgctaatgaacctcagcttgtctcgcctcctgtcttctcaacttacacaactgagccttccacagcaccattgacccctccacctgaactagctcatgcaaccactccctcgtccccagatgttccatatgcccggtttctttcttcttctatggatatcaaaactgctagtaaggagcataacatggctttcttatcaacaacctattctggtggttcaggactccaggcatcataccctctttaccctgagagcccttgtagcagcctcatatcacctgcttctgcaactccgaggactggtctttcctcaccaatacctgaacaagaggtccctacagcccattggaagacttctaggtctgcctgtgacacaccatattccagggcttcacccatccctgagcaagaagccactgcgcagtggaagacttctagatcagcatgtgatacaccttatgccagagcttcaccatcaaacatctttgggctggattcatctgcttctagaaactatttgttagatggcaacttcttccggccagctgcttctgctcaattctacctggaccaggctcagcagacatatccatataatggtgggaggcgtagtgtctcacgggacaagcaagacgcagatgaagttgaagcttacagagcttcgtttggctttagtgcagatgaaatcatgcaaactcaatcttatgtggagataccagatgcacttgatgaatcattcagtatatcaccatttggaaataatgctcctgctactgaggtgtccccatttaatgatctgcccaatgaggttcagaaggctgagaagtcaagtccaaagaaatcagcagatcagatttccaatggttctccacatagagtgctgcacgttgacatattcaagggtaaatgtccatactttttgcatcaaccattcagttgtaatctaggcgtagtagaattactccttttgttatccttgctatctactgcacctaactggtaacttccaatgctaataacctgaactctgggccattggtattgtgtttctgtttacgtaaatgcttatcatgcttatgtgctcacatttcaggaacaaaaggaggacatccgtttgagtacgaggggattgtgaaagatggccatcctttcagaaagacaagggatgagatatctctaaaacccatagaagtaaggaagaaatctccacctggtcattcatgctcagatgctgaaatcgagtacagaagggcaaggagtctgagggaagccaacggtgtcccctcgtggcgcagcacgttgtcgagacagctgcagtga

SEQ ID NO: 27 - DNA, 2502 bp, synthetic construct
  source 1..2502: mol_type = other DNA
atggcttcagctgggagcagcagtggaggtgcaggtggaggcagcagcatcagggcggcgaatggcgcagctgccatcagtgcggccgcgacggcagtaggctcagccgacgccagattccactcccacccaccacagcaggacagggtatatacactacacgacttcttgttccatggcgcatgcaattgctcatgctttagctgcagcttttacccttcttgctttagatgtgtggatgagtaccagcaatttctagtaggccttgacgacctagattgtcacaggctttgcgtacttagtacgaatggtactgcccagtggtggttgcagcttgcaaccccggcatctttacccccaataattaggcttcagtactgtctgcttgtttgcttctagtactgaatagtttgttcagctgaattttccgttttgctgttgatatttgattattacgcgatttgcactccctctatgtttttccccccggatcctagatgactgtactcttaccataacactcgtccagagacctatatgttgtgtaactgttgatttgaccttgcatacacttccacacgtcgtgtcagttgatgtcatgagcaaactaactatgctgggttgattccttcttgcatgatggatctttttccaaatgaattttcagattcctgtaaaatgcttggtggtgtagctattaatgtcaacacatttcttatattcttaagtaaaattggtttagattaatactccaaggaaatttcgttgttgaacaactagtcatacattttttcccctaccaacagctatgtacttgatactaatatgtgcatctctgatctaaacagcggagtagatgggctggctgcttgtcgggtctttcatgtttcggatctcagaagggcgggaagaggattgttcctgccgcacgtactcctgatgggaatggatcaagtgctcgtggaaatggtcatcagtccggttctaattcaaaccaaaatgtgcctttaaatctgtctcttctggctccaccatcctcaccggcatccttctcaaattctgcacttccttcaactgttcaatcgcctaataactttctgtcaatctcagcaaattctcctggtggtccaacatctaatatgtttgctgttgggccatatgctaatgaacctcagcttgtctcgcctcctgtcttctcaacttacacaactgagccttccacagcaccattgacccctccacctgaactagctcatgcaaccactccctcatctccagatgttccatacgctcggtttctttcttcttttatggatatcaaaactgctagtaaggagcataacatggctttcttatcaacaacatattctggtggttcaggactccaggcatcataccctctttaccctgagagcccttgtagcagcctcatatcacctgcttctgctactccgaggactggtctttcctcaccaatacctgaacaagaggtccctactgcccattggaagacttctaggtctgcctgtgaacacaccatattccagggcttcacccatccctgagcaagaagccactgcgcagtggaagacttctagatcggcatgtgatacgccttatgccagggcttcaccatcaaacatctttgggctggactcatctgcttctagaaactatttgttagatggcaacttcttccggccagctgcttctgctcaattctacctggaccaggctcagcagacatatccatataatggtgggaggcgtagtgtctcacgggacaagcaagatgcagatgaagttgaagcttacagagcttcgtttggctttagtgcagatgaaatcatgcaaactcaatcttatgtggagataccagatgcacttgatgaatcattcagtatatcgccatttggaaataatgctcctgctactgaggtgtccccatttaatgatccacccaacgaggttcagaaggctgagaagtcaagtccaaagaaaacggcagatcagatttccaatggctctccacatagagtgctgcacattgacatattcaagggtaaatgtccatactttttgcaccaaccattcagttgtaatctaggcatagtagaattactcttgtttatccttgctatctactgcacctaacaaagctttggtaactttcaatgctaataacctgaactctgggccatttgtcatcagcacttattggtattgtgtttctatttatgtaaatgcttattatgcttatgtgctcgcatttcaggaacaaaaggaggacatccgtctgagtacgagggtattgtgaaagatggccatcctttcagaaagacaagggacgaaatatctctaaaacccatagaagtaaggaagaaatctccacctgggcattcatgctcagatgctgaaattgagtacagaagggcaaggagtctgagggaagccaatggtgtcccctcgtggcgcagcacgttgtcaagacagctgcagtga

SEQ ID NO: 28 - DNA, 2562 bp, synthetic construct
  source 1..2562: mol_type = other DNA
atggcttcagctgggagcagcagtggaggtgcaggtggaggcagcagcatcagggcggcgaatggcgcagctgctatcagtgcggccgcgacggcagtaggctcagccgacgccagattccactcccacccaccacaacaggacagggtatatacactacacgacttcttgttccatggcgcatgcaattgctcatgctttggctgcagcttttacccttcttgctttagatgtatggatgagtaccagcaatttccagtaggccttgacgacctagattgtcacaggctttgcgtacttagtacgaatggtactgcccagtggtggttgcagcttgcaacccccggcatctttacccccaataattaggcttcagtactgtctgcttgtttgcttctactgaataatttgttcggctgaatttttcgttttgctgtcgatattgattgcacgccatttgcactccctctatgtactcttaccagatgactgtttgtactcttaccataacactcctccagagatcctagatgctgtataactgttgatttgaccttggatacacttccacacgttgtgtcagttgatgtcatgagcaaactaactatgctgggctgaaatcttcttgcatgataaatctttttccatatgaaaattttcagatggctgtaaaattcttggtggtgtagctattattgtcaacaaatttcttatattctttagtaaacttgttttttatgcttacccgttatagtaagtttggcccagtttcccttcccatgattttaatgaataaatcactttttctgtttgtagcgagtatagattaatactctgaggaaatttcgttgttgaacatttagtcatacatttcctccctaccaacagctatgtacttgatactaatatgtgcatctctgatctaaacagcggagtagatgggctggctgcttttcgggcctttcatgtttcggatctcagaagggcgggaagaggattgttccggccgcacgtactcctgatgggaatggatcaagtgctcgtggaaatggtcatcagtctggttctaattcaaaccaaaatgtgcctttaaatctgtcgcttctggctccaccatcgtcaccggcatccttctcaaattctgcacttccttcaactgttcagtcgccaaataactttctgtcaatctcagcaaattctcctggtggtccgacatctaatatgtttgctgttgggccatatgctaatgaacctcagcttgtctcgcctcctgtcttctcaacttacacaactgagccttccacagcaccattgacccctccacctgaactagctcatgcaaccactccctcgtccccagatgttccatatgcccggtttctttcttcttctatggatatcaaaactgctagtaaggagcataacatggctttcttatcaacaacctattctggtggttcaggactccaggcatcataccctctttaccctgagagcccttgtagcagcctcatatcacctgcttctgcaactccgaggactggtctttcctcaccaatacctgaacaagaggtccctacagcccattggaagacttctaggtctgcctgtgaacacaccatattccagggcttcacccatccctgagcaagaagccactgcgcagtggaagacttctagatcagcatgtgatacaccttatgccagagcttcaccatcaaacatctttgggctggattcatctgcttctagaaactatttgttagatggcaacttcttccggccagctgcttctgctcaattctacctggaccaggctcagcagacatatccatataatggtgggaggcgtagtgtctcacgggacaagcaagacgcagatgaagttgaagcttacagagcttcgtttggctttagtgcagatgaaatcatgcaaactcaatcttatgtggagataccagatgcacttgatgaatcattcagtatatcaccatttggaaataatgctcctgctactgaggtgtccccatttaatgatctgcccaatgaggttcagaaggctgagaagtcaagtccaaagaaatcagcagatcagatttccaatggttctccacatagagtgctgcacgttgacatattcaagggtaaatgtccatactttttgcatcaaccattcagttgtaatctaggcgtagtagaattactccttttgttatccttgctatctactgcacctaactggtaacttccaatgctaataacctgaactctgggccattggtattgtgtttctgtttacgtaaatgcttatcatgcttatgtgctcacatttcaggaacaaaaggaggacatccgtttgagtacgaggggattgtgaaagatggccatcctttcagaaagacaagggatgagatatctctaaaacccatagaagtaaggaagaaatctccacctggtcattcatgctcagatgctgaaatcgagtacagaagggcaaggagtctgagggaagccaacggtgtcccctcgtggcgcagcacgttgtcgagacagctgcagtga

SEQ ID NO: 29 - protein, 1349 aa, Eragrostis tef
MSSSDPEEIRGRVVVLGADADELARPELEAFHLPSPTLAQEAGSVGGPGPAAAAPVVAPLETELPPNATMPSSSSASSNSNSNEQNKEQENTKKKGVSLAPAPLGSLFRFADGLDCVLMSVGTLGALVHGCSLPVFLRFFADLVDSFGSHADDPDTMVRLVAKYALYFLVVGAAIWASSWAEISCWMWTGERQSTRMRIRYLESALRQDVSFFDTDVRTSDVIYAINADAVIVQDAISEKLGNLIHYMATFVAGFVVGFTAAWQLALVTLAVVPLIAVIGGLSAAALAKLSSRQDALAEASNIAEQAVAQIRTVQAFVGEERAMRAYSLALAAAQRIGYRSGFAKGLGLGGTYFTVFCCYALLLWYGGLLVRRHHTNGGLAIATMFSVMIGGLALGQSAPSMAAFAKARVAAAKIFKIIDHKPLSVVVHGDDDVQLPSVTGRVEMRGVDFAYPSRPDVPVLRGFSLTVPPGKTIALVGSSGSGKSTVVSLIERFYDPSAGEILLDGHDLKSLNRWLRQQIGLVSQEPTLFATSIKENLLLGRDSHSATLAEMEEAARVANAHSFIIKLPDGYDTQVGERGLQLSGGQKQRIAIARAMLKNPGILLLDEATSALDSESEKLVQEALDRFMIGRTTLVIAHRLSTIRKADLVAVLHGGAVSEIGTHEELMGKGEDGAYARLIRMQEQAAQEVAARRSSARNSVSARNSVSSPIMTRNSSYGRSPYSRRLSDFSNADFHYHGGGELPEGNTKKMIHQRVAFRAGASSFLRLAKMNSPEWGYALVGSLGSMVCGSFSAIFAYVLSAVLSVYYAPDPGHMRREIAKYCYLLMGMSSAALVCNTVQHVFWDTVGENLTKRVRERLGAVLRNEMAWFDAEENASARVAARLALDAQNVRSAIGDRISVIVQNSALLLVACTAGFVLQWRLALVLLGVFPLVVAATVLQKMFMKGFSGDLEAAHARATQIAGEAVANLRTVAAFNAEAKIAGLFAGNLRGPLRRCLWKGQVAGCGYGVAQFLLYASYALGLWYAAWLVKHGVSDFSRTIRVFMVLMVSANGAAETLTLAPDFVRGGRAMRSVFETIDRRTEADPDDPDAAPLQLPLLTGELRHVDFCYPSRPEVQVLQDLSLRARAGKTLALVGPSGCGKSSVLALIQRFYEPTSGRVLDGRDARKYNLRALRRAVAVVPQEPFLFAASIHDNIAYGREGGATEAEVLEAATQANAHKFISALPDGYRTQVGERGVQLSGGQRQRIAVARALVKQAAVLLLDEATSALDAESERSVQQALDRHAKTRSTTTIVVAHRLATVRNAHTIAVIDEGKVVEQGSHSHLLNHHPDGTYARMLQLQRLTSSTS

SEQ ID NO: 30 - DNA, 5181 bp, Eragrostis tef
  source 1..5181: mol_type = other DNA
atgtctagcagcgacccggaggagatccgggggcgcgtggtcgtcctcggcgccgatgcggacgagttggctcgccccgagctggaggccttccacctcccgtctccgacactagctcaggaggcaggatctgtcggaggcccaggccctgctgctgctgctcccgtggtggcgccattggagacggagctgcctcccaatgccaccatgccttcttcttcctccgcctcctcgaacagcaacagcaatgagcagaacaaggagcaggagaatacgaagaagaagggggtgtctttggcgcctgctcctcttggttcgctgttccgtttcgccgacggtctggactgcgttctgatgtcggtgggcacgttgggcgcgcttgtccacggttgctccctcccggtgttcctccgcttcttcgcggacctcgtcgactcgttcggctcccacgccgacgacccggacaccatggtgcggctggtggccaagtacgcgctctacttcctggtggtgggcgcggccatctgggcgtcctcctgggcggagatctcgtgctggatgtggaccggcgagcggcagtcgacgcggatgcggatccggtacctggagtcggcgctccggcaggacgtgtccttcttcgacaccgacgtccgcacctccgacgtgatctacgccatcaacgcggacgccgtgatcgtgcaggacgccatcagcgagaagctcggcaacctcatccactacatggccaccttcgtggccgggttcgtggtcgggttcacggcggcatggcagctggcgctggtgacgctggccgtggtgccgctcatcgccgtgatcggcggcctcagcgcggcggcgctggcgaagctgtcgtcgcggagccaggacgcgctggcggaggcgagcaacatcgcggagcaggcggtggcgcagatacggacggtgcaggccttcgtcggggaggagcgcgcaatgcgggcctactcgttggcgctggccgcggcgcagaggatcggataccgcagcggcttcgccaagggcctgggcctgggcggcacctacttcaccgtcttctgctgctacgcgctgctgctctggtacggcggcctcctcgtgcgacgccaccacaccaacggcggcctcgccatcgccaccatgttctccgtcatgatcgggggcctcgcgctgggccagtcggcgcccagcatggccgccttcgcaaaggcgcgcgtcgccgcggccaagatcttcaaaatcatcgaccacaagcccctctccgtcgtcgtccatggcgacgacgatgtccaactcccttccgtgacggggcgggtggagatgcggggcgttgacttcgcctacccgtcgcggccggacgtccccgtcctgcgcggcttctccctcaccgtgccgcccggcaagaccatcgcgctcgtcgggagctcaggctccgggaagagcacggtcgtctccctcatcgagaggttctacgacccaagtgcaggtacacaactacttaacgtcttagaagtgtctgtcagtcaattgtcttcttcattcacaatcgtattcattgattgaaagaaaaatatgcttctcaggcgagattctgcttgacgggcacgacctcaagtccctcaacctgcggtggctccggcagcagatcgggctggtgagccaggagccgacgctgttcgccaccagcatcaaggagaacctgctgctgggccgggacagccacagcgccaccctcgccgagatggaggaggccgcaagggtcgccaacgcccactcattcatcatcaagctgcccgacggatacgacacccaggtatgtactagcaccgttgctttcctttcacattcctctcctctcctgagcaactagatatgtactagcaccattgctccatctcaatgccaccatcctcaaaaaagaaggaaagcttttctccatctcatctccatcatctcattcctggcgctcgtgccctgcctcagatccatgggcgcaagtggacgggcacgcacgagacgtggctagtagtactccagtatagcaatgcttattagcctcactcactcaccagtgctatactagtagggcagccagcagcaacacatgacacgggaggagacagcaccacccggccggggtccctcccattgttaggttaggagcctgttgctgtcacctgctgttgcgagttaacaacatgtaacatgctaaagattcgttcttccttcctttccacaacacaggtcgctttctgacttgcttgcttctcgttctcgacacaatgcaatgcagcagctagcagcccatcacctagtcctcacccatctttctgtagtactgcttccattttcaccatggcttggctcaccggccggggtggtagtagaaagtagaattcccaggaatctagtaagagaacaagtcccaagttggcattttgtcaggaatgtcaagttgcgacttgcgagatcccatcccaagaattttctgcaatgggagaggagaggaccatggccgttgtgtttattttactgtcaagcgcaggcatagggcggttcctccttccttgagtcattaacacaaatcctgcgtggagtgtattaacaagagtttgtagcactaccaaaccacatgggccactgactctgcagctgaggagtagtaaaatagtgcagtgtggaggccggcccatcctcctatgcacatgacatgatccgacggagatagtagaatagatccgacatgatattttttttaattcatgacaatcatgataaaaaaaaagcaaagtcaaacaaggtcaattatgataaaaaaaatgcaatgtgcaggtcggtgagcgcggtctgcagctgtctggcggtcagaagcagcgcatcgccattgcccgcgccatgctcaagaaccccggcatcttgctgctggacgaggcgacgagcgcgctggactctgagtcggagaagctggtgcaggaggcgctggaccggttcatgatcgggcgcacgaccctggtgatcgcgcaccggctgtcgacgatccgcaaggccgacctggtggcggtgctgcacggcggcgccgtgtcggagattgggacgcacgaggagctgatgggcaagggcgaggacggcgcgtacgcgcggctgatccggatgcaggagcaggccgcgcaggaggtggccgcccgtcggagcagcgcgcgcaactcggtgagcgcgcgcaactcggtgagctcgcccatcatgacgcgcaactcctcctacgggcggtcgccctactcgcgccgcctctccgacttctccaacgccgacttccactaccacggcggtggtgaactaccggagggtaataccaagaagatgattcatcagagggttgcgttccgggcgggggcgagctctttcctgcgtctggcgaagatgaactcgcccgagtgggggtacgctctggtgggctcgctgggctccatggtgtgcggctccttcagcgccatcttcgcctacgttctgagcgccgtgctgagcgtgtactacgcccccgacccgggccacatgcggcgggagatcgccaagtactgctacctgctgatgggcatgtcttccgcggcgctggtgtgcaacacggtgcagcacgtgttctgggacacggtgggcgagaacctgacgaagcgcgtccgggagcgcatgctcggcgccgtgctccgcaacgagatggcgtggttcgacgccgaggagaacgccagcgcccgggtggccgcgaggctggcgctggacgcgcagaacgtgcgctccgccatcggcgaccgcatctcggtgatcgtgcagaactcggcgctgctgctggtggcgtgcacggccgggttcgtgctgcagtggcgcctcgccctggtgctgctgggcgtgttcccgctggtggtggccgccaccgtgctgcagaagatgttcatgaagggcttctcgggggacctggaggcggcccacgcccgcgccacgcagatcgccggcgaggccgtcgccaacctgcgcaccgtcgccgcgttcaacgccgaggccaagatcgcgggactcttcgcgggcaacctgcggggcccgctgcggcggtgcctctggaagggccaggtggccgggtgcggctacggggtggcgcagttcctgctgtacgcgtcctacgcgctggggctctggtacgcggcgtggctggtgaagcacggcgtctccgacttctcccgcaccatccgcgtcttcatggtgctcatggtctccgccaacggcgccgccgagacgctcacgctggcgccggacttcgtcaggggcggacgcgccatgcgctccgtcttcgagaccatcgacaggcgcaccgaggcggaccccgacgacccggacgcggcgccattgcaactgccattattgacaggggtggagctgcggcacgtggacttctgctacccgtcgcggccggaggtgcaggtgctgcaggacctcagcctgcgcgcgcgcgcggggaagacgctggcgctcgtggggcccagcggctgcggcaagagctccgtgctggcgctcatccagcgcttctacgagcccacctcgggccgcgtcctcctcgacggccgggacgcgcgcaagtacaacctccgggcgctgcgccgcgccgtcgccgtggtgccgcaggagcccttcctgttcgcggcgagcatccacgacaacatcgcctacggccgcgaggggggcgccacggaggcggaggtgctggaggcggccacgcaggccaacgcgcacaagttcatctccgcgctgcccgacggctacaggacgcaggtcggggagcgcggggtgcagctctccggcggccagcgccagcgcatcgccgtggcgcgcgccctcgtcaagcaggccgccgttctgctgctggacgaggccaccagcgcgctggacgccgagtcggagcgcagcgtgcagcaggcgctggaccgccacgccaagacccgcagcaccaccaccatcgtcgtcgcgcaccgcctcgccaccgtgcgcaacgcccacaccatcgccgtcatcgacgagggcaaggtcgtcgagcagggatcgcactcgcacctgctcaaccaccacccggacggaacctacgcgcgcatgctccaactacagcgactcacctcatccacttcctaa

SEQ ID NO: 31 - protein, 1346 aa, Eragrostis tef
MSASSDPEEIRGRVVVLGADADELARPELEAFHLPSPTLEEAGASVGGPAAANPPVVAPLETELPPNAMPSSSSSSNSNSNEQNKEEKKKGVALAPAPLGSLFRFADGLDCVLMSVGTLGALVHGCSLPVFLRFFAELVDSFGSHADDPDTMVRLVARYALYFLVVGAAIWASSWAEISCWMWTGERQSTRMRIRYLESALRQDVSFFDTDVRTSDVIYAINADAVIVQDAISEKLGNLIHYMATFVAGFVVGFTAAWQLALVTLAVVPLIAVIGGLSAAALAKLSSRSQDALAEASNIAEQAVAQIRTVQAFVGEERAMRAYSLALGMAQRIGYRSGFAKGLGLGGTYFTVFCCYALLLWYGGHLVRRHHTNGGLAIATMFSVMIGGLALGQSAPSMAAFAKARVAAAKIFRIIDHLAVVHGDHVQLPSVTGRVEMRGVDFAYPSRPDIPVLRGFSLTVPPGKTIALVGSSGSGKSTVVSLIERFYDPSAGEILLDGHDLKSLNLRWLRQQIGLVSQEPTLFATSIKENLLLGRDSQSATLAEMEEAARVANAHSFIIKLPDGYDTQVGERGLQLSGGQKQRIAIARAMLKNPGILLLDEATSALDSESEKLVQEALDRFMIGRTTLVIAHRLSTIRKADLVAVLHGGAVSEIGTHDELMAKGEDGAYARLIRMQQEQAAAQEVAARRSSARPSSARNSVSSPIMTRNSSYGRSPYSRRLSDFSNADSHHYYHGGELIESNNKAHHQRRIAFRAGASSFWRLAKMNSPEWGYALVGSLGSMVCGSFSAIFAYALSAVLSVYYAPDPGYMRRQIGKYCYLLMGMSSAALVFNTVQHAFWDTVGENLTKRVRERMFGAVLRNEVAWFDAEENASARVAARLALDAQNVRSAIGDRISVIVQNSALLLVACTAGFVLQWRLALVLLAVFPLVVAATVLQKMFMKGFSGDLEAAHARATQIAGEAVANLRTVAAFNAQAKIAGLFAANLRGPLRRCFWKGQAAGCGYGLAQFLLYASYALGLWYAAWLVKHGVSDFSRAIRVFMVLMVSANGAAETLTLAPDFVKGGRAMRSVFETIDRRTETEPDDPDAAPLPSDAVSVELRHVDFCYPSRPDVRVLQDLSLRARAGKTLALVGPSGCGKSSVLALIQRFYEPTSGRVLLDGRDARKYNLRALRRAIAVVPQEPFLFAATIHDNIAYGREGATEAEVVEAATQANAHKFISALPDGYRTQVGERGVQLSGGQRQRIAVARALVKQAAVLLLDEATSALDAESERSVQQALDRHAKTRSTTTIVVAHRLATVRDAHTIAVIDDGKVVEQGSHSHLLNHHPDGTYARMLHLQRLTAPSTS

SEQ ID NO: 32 - DNA, 5238 bp, Eragrostis tef
  source 1..5238: mol_type = other DNA
atgtcagctagtagcgacccggaggagatccggggccgcgtggtggtcctcggcgccgatgccgacgagttggctcgccccgagctggaggccttccacctcccatctccgacattagaggaggcaggagcctctgtcggaggccctgctgctgctaatcctccggtggtggcgccattggagacggagctgcctcccaatgcgatgccttcttcttcctcctcctccaacagcaacagcaatgagcagaacaaggaggagaagaagaagggggtggcgttggcgcctgctcctcttgggtcgctgttccgtttcgccgacggtcttgactgcgttctgatgtcggtgggcacgttgggcgcgcttgtccacggctgctccctccccgtgttcctccgcttcttcgcggagctggtggactcgttcggctcccacgccgacgacccggacaccatggtgcggctggtggccaggtacgcgttgtattttctggtggtgggcgcggccatctgggcgtcctcctgggcggagatctcgtgctggatgtggacgggggagcggcagtcgacgcggatgcggatccggtacctggagtcggcgctccgtcaggacgtgtccttcttcgacaccgacgtccgcacctccgacgtgatctacgccatcaacgccgacgccgtcatcgtgcaggacgccatcagcgagaagctcggcaacctcatccactacatggccaccttcgtggccggattcgtggtggggttcacggccgcctggcagctggcgctggtgacgctggccgtggtgccgctcatcgccgtgatcggcggcctcagcgcggcggcgctggcgaagctgtcgtcgcggagccaggacgcgctggcggaggcgagcaacatcgcggagcaggcggtggcgcagatacggacggtgcaggcgttcgtcggggaggagcgcgcaatgcgggcctactcgttggctctaggaatggcgcagaggatcgggtaccgcagcggcttcgccaagggcctgggcctgggcggcacctacttcaccgtcttctgctgctacgcgctgctgctctggtacggcggccacctcgtgcgccgccaccacaccaacggcggcctcgccatcgccaccatgttctccgtcatgatcgggggcctcgcgctgggccagtccgcgcccagcatggccgccttcgcaaaggcgcgcgtcgccgcggccaagatcttcaggatcatcgaccaccttgccgtcgtccatggcgaccatgtccagctcccttccgtgacggggcgggtggagatgcggggcgttgacttcgcctacccgtcgcgaccggacatccccgtcctgcgcggcttctccctcaccgtgccgccaggcaagaccatcgcgctcgtcgggagctcaggctccggaaagagcacagtcgtctccctcattgagaggttctacgacccaagtgcaggtacacaaaaaaacttccttggttgaaaatttctcttgttcgttcgttcacacacattcatcgattcaaaaacatatgcttcttactaattactactactaggtgagattctgcttgacgggcacgacctcaagtccctgaacctgcgctggctccggcagcagatcgggctggtgagccaggagccgacgctgttcgccacgagcatcaaggagaacctgctgctgggccgcgacagccagagcgccaccctcgccgagatggaggaggccgccagggtcgccaacgcccactcattcatcatcaagctgcccgacggctacgacacccaggtatgtacagtatgcttcgctttgctccatctcactcaatgccaccctcggaaaggaagaaaagcttttcctccatcatctcattccttcctggcgctctcagatccacgggcacgcacgagactgggcgggctactagagtactaccagtatactagcaatgcttgacctcactcaccagtactagtactctagtagggcagcaacacatgacacgggagacagcaccacccggccggggtccctcccattgttaggttaggaggctgttgctgtcacctgttgttgcgagttactcctaacaacatgtaacatgctaaagattctttcttcctttctttccacaacacaggtcgctttctgagttgcttgcttctcgacacaatgcaatgcagcagcagcagcagctagcagcccatcactcatctttttgtgctgcattcattttcaccatggcttgcctcaaaaaagaagctactcactccagtaaatcatccctctcgtggtggtagtactccaaagtagaattcccaggaatctagtaacggaacaagtcccaagttggcattttgtcaggaatgtcaagttgcgagatcccatcccaagaattttgtggagagagagagagagagagagggagcaatgggagaggaggaggaccatggccgttctgttgttattttagtgtcaagtgcaggcagagtgcagttaacaaatcctgagtagtttattaacaagagtttgtactagtaccaaaccacatggggcgccactgactctgcattgctagttaaaaaatattgcagtgtggtggaggccgggccggcccagcccatcctcctgtgcacatggcatgatccgacggagatagatcctgcattagtttagtttaaacctttctttctgttctttcttctagtagtagtaggagtatcccagtcaaactaagcactctgctggtcaaaaaaaaaaaaaaacaggcggagtactcctgatttaattaactgatgatgatgatgacatttgccttaagttagtaattcatgatgagaataatagaaggaaaaacaaagtcaatggatgtgcatgcaggtcggtgagcgcggtctgcagctgtctggcggtcagaagcagcgcatcgccatcgcgcgcgccatgctcaagaaccctgggatcttgctgctggacgaggcgacgagcgcgctggactcggagtccgagaagctggtgcaggaggcgctggaccggttcatgatcgggcgcacgaccctggtgatcgcgcaccggctgtcgacgatccgcaaggccgacctggtggccgtgctgcacggcggcgccgtgtcggagattgggacgcacgacgagctgatggccaagggcgaggacggcgcgtacgcgcggctgatccggatgcagcaggagcaggccgccgcgcaggaggtggccgcccgtcggagcagcgcgcggccgtcgagcgcgcgcaactcggtcagctcgcccatcatgacgcgcaactcctcctacgggcggtcgccctactcgcggcgcctctccgacttctccaacgccgactcccaccactactaccatggtggtgaacttattgagagtaacaacaaggctcatcatcaaaggaggaggattgcgttccgggctggggcgagctcgttctggaggctggccaagatgaactcgcccgagtgggggtacgcgctggtggggtccctgggctccatggtgtgcggctccttcagcgccatcttcgcctacgcgctgagcgccgtgctgagcgtgtactacgcccccgacccgggttacatgcggcggcagatcggcaagtactgctacctgctgatgggcatgtcttcggcggcgctggtgttcaacacggtgcagcacgcgttctgggacacggtgggcgagaacctgacgaagcgcgtccgggagcgcatgttcggcgccgtgctccgcaacgaggtggcgtggttcgacgccgaggagaacgccagcgcccgggtggccgccaggctggcgctggacgcgcagaacgtgcgttccgccatcggcgaccgcatctcggtgatcgtgcagaactcggcgctgctgctggtggcctgcacggcggggttcgtgctgcagtggcgcctggcgctggtgctcctggccgtgttcccgctggtggtggccgccaccgtgctgcagaagatgttcatgaagggcttctcgggcgacctggaggcggcccacgcccgcgccacgcagatcgccggcgaggccgtcgccaacctgcgcaccgtcgccgccttcaacgcccaggccaagatcgcgggcctcttcgccgccaacctgcggggcccgctgcggcgctgcttctggaagggccaggccgccggctgcggctacgggctggcccagttcctgctctacgcgtcctacgcgctgggcctctggtacgcggcctggctcgtcaagcacggcgtctccgacttctcccgcgccatccgggtcttcatggtgctcatggtctccgccaacggcgccgccgagacgctcaccctggcgccggacttcgtcaaaggcggccgcgccatgcgatccgtcttcgagaccatcgacaggcgcaccgagacggagcccgacgacccggacgccgcgccactgccatcggacgcggtctccgtggagctgcggcacgtggacttctgctacccgtcgcggccggacgtgcgggtgctgcaggacctcagcctgcgcgcgcgcgcggggaagacgctggcgctcgtggggcccagcgggtgcggcaagagctccgtgctcgcgctcatccagcgcttctacgagcccacctcgggccgcgtcctcctcgacggccgcgacgcgcgcaagtacaacctccgggcgctgcggcgcgccatcgccgtcgtgccgcaggagcccttcctcttcgcggccaccatccacgacaacatcgcctacggccgcgagggcgccacggaggcggaggtggtggaggcggccacgcaggccaacgcgcacaagttcatctccgcgctgcccgacgggtacaggacgcaggtcggcgagcgcggggtgcagctctccggcgggcagcgacagcgcatcgccgtggctcgggcgcttgtcaaacaggccgccgttctgctcctcgacgaggccaccagcgcgctggacgccgagtcggagcgcagcgtgcagcaggcgctggaccgccacgccaagacccgtagcaccaccaccatcgtcgtcgcgcaccgcctcgccaccgtccgcgacgcacacaccatcgccgtcatcgacgacggcaaggtcgtcgagcagggatcgcactcacacctgctcaaccaccaccccgacggaacctacgcgcgcatgctccacctccagcgactcaccgcgccatccacttcctaa

SEQ ID NO: 33 - DNA, 5182 bp, synthetic construct
  source 1..5182: mol_type = other DNA
atgtctagcagcgacccggaggagatccgggggcgcgtggtcgtcctcggcgccgatgcggacgagttggctcgccccgagctggaggccttccacctcccgtctccgacactagctcaggaggcaggatctgtcggaggcccaggccctgctgctgctgctcccgtggtggcgccattggagacggagctgcctcccaatgccaccatgccttcttcttcctccgcctcctcgaacagcaacagcaatgagcagaacaaggagcaggagaatacgaagaagaagggggtgtctttggcgcctgctcctcttggttcgctgttccgtttcgccgacggtctggactgcgttctgatgtcggtgggcacgttgggcgcgcttgtccacggttgctccctcccggtgttcctccgcttcttcgcggacctcgtcgactcgttcggctcccacgccgacgacccggacaccatggtgcggctggtggccaagtacgcgctctacttcctggtggtgggcgcggccatctgggcgtcctcctgggcggagatctcgtgctggatgtggaccggcgagcggcagtcgacgcggatgcggatccggtacctggagtcggcgctccggcaggacgtgtccttcttcgacaccgacgtccgcacctccgacgtgatctacgccatcaacgcggacgccgtgatcgtgcaggacgccatcagcgagaagctcggcaacctcatccactacatggccaccttcgtggccgggttcgtggtcgggttcacggcggcatggcagctggcgctggtgacgctggccgtggtgccgctcatcgccgtgatcggcggcctcagcgcggcggcgctggcgaagctgtcgtcgcggagccaggacgcgctggcggaggcgagcaacatcgcggagcaggcggtggcgcagatacggacggtgcaggccttcgtcggggaggagcgcgcaatgcgggcctactcgttggcgctggccgcggcgcagaggatcggataccgcagcggcttcgccaagggcctgggcctgggcggcacctacttcaccgtcttctgctgctacgcgctgctgctctggtacggcggcctcctcgtgcgacgccaccacaccaacggcggcctcgccatcgccaccatgttctccgtcatgatcgggggcctcgcgctgggccagtcggcgcccagcatggccgccttcgcaaaggcgcgcgtcgccgcggccaagatcttcaaaatcatcgaccacaagcccctctccgtcgtcgtccatggcgacgacgatgtccaactcccttccgtgacggggcgggtggagatgcggggcgttgacttcgcctacccgtcgcggccggacgtccccgtcctgcgcggcttctccctcaccgtgccgcccggcaagaccatcgcgctcgtcgggagctcaggctccgggaagagcacggtcgtctccctcatcgagaggttctacgacccaagtgcaggtacacaactacttaacgtcttagaagtgtctgtcagtcaattgtcttcttcattcacaatcgtattcattgattgaaagaaaaatatgcttctcaggcgagattctgcttgacgggcacgacctcaagtccctcaacctgcggtggctccggcagcagatcgggctggtgagccaggagccgacgctgttcgccaccagcatcaaggagaacctgctgctgggccgggacagccacagcgccaccctcgccgagatggaggaggccgcaagggtcgccaacgcccactcattcatcatcaagctgcccgacggatacgacacccaggtatgtactagcaccgttgctttcctttcacattcctctcctctcctgagcaactagatatgtactagcaccattgctccatctcaatgccaccatcctcaaaaaagaaggaaagcttttctccatctcatctccatcatctcattcctggcgctcgtgccctgcctcagatccatgggcgcaagtggacgggcacgcacgagacgtggctagtagtactccagtatagcaatgcttattagcctcactcactcaccagtgctatactagtagggcagccagcagcaacacatgacacgggaggagacagcaccacccggccggggtccctcccattgttaggttaggagcctgttgctgtcacctgctgttgcgagttaacaacatgtaacatgctaaagattcgttcttccttcctttccacaacacaggtcgctttctgacttgcttgcttctcgttctcgacacaatgcaatgcagcagctagcagcccatcacctagtcctcacccatctttctgtagtactgcttccattttcaccatggcttggctcaccggccggggtggtagtagaaagtagaattcccaggaatctagtaagagaacaagtcccaagttggcattttgtcaggaatgtcaagttgcgacttgcgagatcccatcccaagaattttctgcaatgggagaggagaggaccatggccgttgtgtttattttactgtcaagcgcaggcatagggcggttcctccttccttgagtcattaacacaaatcctgcgtggagtgtattaacaagagtttgtagcactaccaaaccacatgggccactgactctgcagctgaggagtagtaaaatagtgcagtgtggaggccggcccatcctcctatgcacatgacatgatccgacggagatagtagaatagatccgacatgatattttttttaattcatgacaatcatgataaaaaaaaagcaaagtcaaacaaggtcaattatgataaaaaaaatgcaatgtgcaggtcggtgagcgcggtctgcagctgtctggcggtcagaagcagcgcatcgccattgcccgcgccatgctcaagaaccccggcatcttgctgctggacgaggcgacgagcgcgctggactctgagtcggagaagctggtgcaggaggcgctggaccggtttcatgatcgggcgcacgaccctggtgatcgcgcaccggctgtcgacgatccgcaaggccgacctggtggcggtgctgcacggcggcgccgtgtcggagattgggacgcacgaggagctgatgggcaagggcgaggacggcgcgtacgcgcggctgatccggatgcaggagcaggccgcgcaggaggtggccgcccgtcggagcagcgcgcgcaactcggtgagcgcgcgcaactcggtgagctcgcccatcatgacgcgcaactcctcctacgggcggtcgccctactcgcgccgcctctccgacttctccaacgccgacttccactaccacggcggtggtgaactaccggagggtaataccaagaagatgattcatcagagggttgcgttccgggcgggggcgagctctttcctgcgtctggcgaagatgaactcgcccgagtgggggtacgctctggtgggctcgctgggctccatggtgtgcggctccttcagcgccatcttcgcctacgttctgagcgccgtgctgagcgtgtactacgcccccgacccgggccacatgcggcgggagatcgccaagtactgctacctgctgatgggcatgtcttccgcggcgctggtgtgcaacacggtgcagcacgtgttctgggacacggtgggcgagaacctgacgaagcgcgtccgggagcgcatgctcggcgccgtgctccgcaacgagatggcgtggttcgacgccgaggagaacgccagcgcccgggtggccgcgaggctggcgctggacgcgcagaacgtgcgctccgccatcggcgaccgcatctcggtgatcgtgcagaactcggcgctgctgctggtggcgtgcacggccgggttcgtgctgcagtggcgcctcgccctggtgctgctgggcgtgttcccgctggtggtggccgccaccgtgctgcagaagatgttcatgaagggcttctcgggggacctggaggcggcccacgcccgcgccacgcagatcgccggcgaggccgtcgccaacctgcgcaccgtcgccgcgttcaacgccgaggccaagatcgcgggactcttcgcgggcaacctgcggggcccgctgcggcggtgcctctggaagggccaggtggccgggtgcggctacggggtggcgcagttcctgctgtacgcgtcctacgcgctggggctctggtacgcggcgtggctggtgaagcacggcgtctccgacttctcccgcaccatccgcgtcttcatggtgctcatggtctccgccaacggcgccgccgagacgctcacgctggcgccggacttcgtcaggggcggacgcgccatgcgctccgtcttcgagaccatcgacaggcgcaccgaggcggaccccgacgacccggacgcggcgccattgcaactgccattattgacaggggtggagctgcggcacgtggacttctgctacccgtcgcggccggaggtgcaggtgctgcaggacctcagcctgcgcgcgcgcgcggggaagacgctggcgctcgtggggcccagcggctgcggcaagagctccgtgctggcgctcatccagcgcttctacgagcccacctcgggccgcgtcctcctcgacggccgggacgcgcgcaagtacaacctccgggcgctgcgccgcgccgtcgccgtggtgccgcaggagcccttcctgttcgcggcgagcatccacgacaacatcgcctacggccgcgaggggggcgccacggaggcggaggtgctggaggcggccacgcaggccaacgcgcacaagttcatctccgcgctgcccgacggctacaggacgcaggtcggggagcgcggggtgcagctctccggcggccagcgccagcgcatcgccgtggcgcgcgccctcgtcaagcaggccgccgttctgctgctggacgaggccaccagcgcgctggacgccgagtcggagcgcagcgtgcagcaggcgctggaccgccacgccaagacccgcagcaccaccaccatcgtcgtcgcgcaccgcctcgccaccgtgcgcaacgcccacaccatcgccgtcatcgacgagggcaaggtcgtcgagcagggatcgcactcgcacctgctcaaccaccacccggacggaacctacgcgcgcatgctccaactacagcgactcacctcatccacttcctaa

SEQ ID NO: 34 - DNA, 5239 bp, synthetic construct
  source 1..5239: mol_type = other DNA
atgtcagctagtagcgacccggaggagatccggggccgcgtggtggtcctcggcgccgatgccgacgagttggctcgccccgagctggaggccttccacctcccatctccgacattagaggaggcaggagcctctgtcggaggccctgctgctgctaatcctccggtggtggcgccattggagacggagctgcctcccaatgcgatgccttcttcttcctcctcctccaacagcaacagcaatgagcagaacaaggaggagaagaagaagggggtggcgttggcgcctgctcctcttgggtcgctgttccgtttcgccgacggtcttgactgcgttctgatgtcggtgggcacgttgggcgcgcttgtccacggctgctccctccccgtgttcctccgcttcttcgcggagctggtggactcgttcggctcccacgccgacgacccggacaccatggtgcggctggtggccaggtacgcgttgtattttctggtggtgggcgcggccatctgggcgtcctcctgggcggagatctcgtgctggatgtggacgggggagcggcagtcgacgcggatgcggatccggtacctggagtcggcgctccgtcaggacgtgtccttcttcgacaccgacgtccgcacctccgacgtgatctacgccatcaacgccgacgccgtcatcgtgcaggacgccatcagcgagaagctcggcaacctcatccactacatggccaccttcgtggccggattcgtggtggggttcacggccgcctggcagctggcgctggtgacgctggccgtggtgccgctcatcgccgtgatcggcggcctcagcgcggcggcgctggcgaagctgtcgtcgcggagccaggacgcgctggcggaggcgagcaacatcgcggagcaggcggtggcgcagatacggacggtgcaggcgttcgtcggggaggagcgcgcaatgcgggcctactcgttggctctaggaatggcgcagaggatcgggtaccgcagcggcttcgccaagggcctgggcctgggcggcacctacttcaccgtcttctgctgctacgcgctgctgctctggtacggcggccacctcgtgcgccgccaccacaccaacggcggcctcgccatcgccaccatgttctccgtcatgatcgggggcctcgcgctgggccagtccgcgcccagcatggccgccttcgcaaaggcgcgcgtcgccgcggccaagatcttcaggatcatcgaccaccttgccgtcgtccatggcgaccatgtccagctcccttccgtgacggggcgggtggagatgcggggcgttgacttcgcctacccgtcgcgaccggacatccccgtcctgcgcggcttctccctcaccgtgccgccaggcaagaccatcgcgctcgtcgggagctcaggctccggaaagagcacagtcgtctccctcattgagaggttctacgacccaagtgcaggtacacaaaaaaacttccttggttgaaaatttctcttgttcgttcgttcacacacattcatcgattcaaaaacatatgcttcttactaattactactactaggtgagattctgcttgacgggcacgacctcaagtccctgaacctgcgctggctccggcagcagatcgggctggtgagccaggagccgacgctgttcgccacgagcatcaaggagaacctgctgctgggccgcgacagccagagcgccaccctcgccgagatggaggaggccgccagggtcgccaacgcccactcattcatcatcaagctgcccgacggctacgacacccaggtatgtacagtatgcttcgctttgctccatctcactcaatgccaccctcggaaaggaagaaaagcttttcctccatcatctcattccttcctggcgctctcagatccacgggcacgcacgagactgggcgggctactagagtactaccagtatactagcaatgcttgacctcactcaccagtactagtactctagtagggcagcaacacatgacacgggagacagcaccacccggccggggtccctcccattgttaggttaggaggctgttgctgtcacctgttgttgcgagttactcctaacaacatgtaacatgctaaagattctttcttcctttctttccacaacacaggtcgctttctgagttgcttgcttctcgacacaatgcaatgcagcagcagcagcagctagcagcccatcactcatctttttgtgctgcattcattttcaccatggcttgcctcaaaaaagaagctactcactccagtaaatcatccctctcgtggtggtagtactccaaagtagaattcccaggaatctagtaacggaacaagtcccaagttggcattttgtcaggaatgtcaagttgcgagatcccatcccaagaattttgtggagagagagagagagagagagggagcaatgggagaggaggaggaccatggccgttctgttgttattttagtgtcaagtgcaggcagagtgcagttaacaaatcctgagtagtttattaacaagagtttgtactagtaccaaaccacatggggcgccactgactctgcattgctagttaaaaaatattgcagtgtggtggaggccgggccggcccagcccatcctcctgtgcacatggcatgatccgacggagatagatcctgcattagtttagtttaaacctttctttctgttctttcttctagtagtagtaggagtatcccagtcaaactaagcactctgctggtcaaaaaaaaaaaaaaacaggcggagtactcctgatttaattaactgatgatgatgatgacatttgccttaagttagtaattcatgatgagaataatagaaggaaaaacaaagtcaatggatgtgcatgcaggtcggtgagcgcggtctgcagctgtctggcggtcagaagcagcgcatcgccatcgcgcgcgccatgctcaagaaccctgggatcttgctgctggacgaggcgacgagcgcgctggactcggagtccgagaagctggtgcaggaggcgctggaccggttacatgatcgggcgcacgaccctggtgatcgcgcaccggctgtcgacgatccgcaaggccgacctggtggccgtgctgcacggcggcgccgtgtcggagattgggacgcacgacgagctgatggccaagggcgaggacggcgcgtacgcgcggctgatccggatgcagcaggagcaggccgccgcgcaggaggtggccgcccgtcggagcagcgcgcggccgtcgagcgcgcgcaactcggtcagctcgcccatcatgacgcgcaactcctcctacgggcggtcgccctactcgcggcgcctctccgacttctccaacgccgactcccaccactactaccatggtggtgaacttattgagagtaacaacaaggctcatcatcaaaggaggaggattgcgttccgggctggggcgagctcgttctggaggctggccaagatgaactcgcccgagtgggggtacgcgctggtggggtccctgggctccatggtgtgcggctccttcagcgccatcttcgcctacgcgctgagcgccgtgctgagcgtgtactacgcccccgacccgggttacatgcggcggcagatcggcaagtactgctacctgctgatgggcatgtcttcggcggcgctggtgttcaacacggtgcagcacgcgttctgggacacggtgggcgagaacctgacgaagcgcgtccgggagcgcatgttcggcgccgtgctccgcaacgaggtggcgtggttcgacgccgaggagaacgccagcgcccgggtggccgccaggctggcgctggacgcgcagaacgtgcgttccgccatcggcgaccgcatctcggtgatcgtgcagaactcggcgctgctgctggtggcctgcacggcggggttcgtgctgcagtggcgcctggcgctggtgctcctggccgtgttcccgctggtggtggccgccaccgtgctgcagaagatgttcatgaagggcttctcgggcgacctggaggcggcccacgcccgcgccacgcagatcgccggcgaggccgtcgccaacctgcgcaccgtcgccgccttcaacgcccaggccaagatcgcgggcctcttcgccgccaacctgcggggcccgctgcggcgctgcttctggaagggccaggccgccggctgcggctacgggctggcccagttcctgctctacgcgtcctacgcgctgggcctctggtacgcggcctggctcgtcaagcacggcgtctccgacttctcccgcgccatccgggtcttcatggtgctcatggtctccgccaacggcgccgccgagacgctcaccctggcgccggacttcgtcaaaggcggccgcgccatgcgatccgtcttcgagaccatcgacaggcgcaccgagacggagcccgacgacccggacgccgcgccactgccatcggacgcggtctccgtggagctgcggcacgtggacttctgctacccgtcgcggccggacgtgcgggtgctgcaggacctcagcctgcgcgcgcgcgcggggaagacgctggcgctcgtggggcccagcgggtgcggcaagagctccgtgctcgcgctcatccagcgcttctacgagcccacctcgggccgcgtcctcctcgacggccgcgacgcgcgcaagtacaacctccgggcgctgcggcgcgccatcgccgtcgtgccgcaggagcccttcctcttcgcggccaccatccacgacaacatcgcctacggccgcgagggcgccacggaggcggaggtggtggaggcggccacgcaggccaacgcgcacaagttcatctccgcgctgcccgacgggtacaggacgcaggtcggcgagcgcggggtgcagctctccggcgggcagcgacagcgcatcgccgtggctcgggcgcttgtcaaacaggccgccgttctgctcctcgacgaggccaccagcgcgctggacgccgagtcggagcgcagcgtgcagcaggcgctggaccgccacgccaagacccgtagcaccaccaccatcgtcgtcgcgcaccgcctcgccaccgtccgcgacgcacacaccatcgccgtcatcgacgacggcaaggtcgtcgagcagggatcgcactcacacctgctcaaccaccaccccgacggaacctacgcgcgcatgctccacctccagcgactcaccgcgccatccacttcctaa

SEQ ID NO: 35 - DNA, 23 bp, synthetic construct
  source 1..23: mol_type = other DNA
ggcccggacttcgagccaatggg

SEQ ID NO: 36 - DNA, 23 bp, synthetic construct
  source 1..23: mol_type = other DNA
gcgacttcttcgaggacagccgg

SEQ ID NO: 37 - DNA, 23 bp, synthetic construct
  source 1..23: mol_type = other DNA
cctggaatatggtgtgtcacagg

SEQ ID NO: 38 - DNA, 23 bp, synthetic construct
  source 1..23: mol_type = other DNA
tcgtgcgcccgatcatgaaccgg

SEQ ID NO: 39 - DNA, 7864 bp, synthetic construct
  source 1..7864: mol_type = other DNA
tgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttcctgcagggcgatctattcgaatggacaagaagtactcgatcggcctcgacatcgggacgaactcagttggctgggccgtgatcaccgacgagtacaaggtgccctctaagaagttcaaggtcctggggaacaccgaccgccattccatcaagaagaacctcatcggcgctctcctgttcgacagcggggagaccgctgaggctacgaggctcaagagaaccgctaggcgccggtacacgagaaggaagaacaggatctgctacctccaagagattttctccaacgagatggccaaggttgacgattcattcttccaccgcctggaggagtctttcctcgtggaggaggataagaagcacgagcggcatcccatcttcggcaacatcgtggacgaggttgcctaccacgagaagtaccctacgatctaccatctgcggaagaagctcgtggactccaccgataaggcggacctcagactgatctacctcgctctggcccacatgatcaagttccgcggccatttcctgatcgagggggatctcaacccagacaacagcgatgttgacaagctgttcatccaactcgtgcagacctacaaccaactcttcgaggagaacccgatcaacgcctctggcgtggacgcgaaggctatcctgtccgcgaggctctcgaagtccaggaggctggagaacctgatcgctcagctcccaggcgagaagaagaacggcctgttcgggaacctcatcgctctcagcctggggctcaccccgaacttcaagtcgaacttcgatctcgctgaggacgccaagctgcaactctccaaggacacctacgacgatgacctcgataacctcctggcccagatcggcgatcaatacgcggacctgttcctcgctgccaagaacctgtcggacgccatcctcctgtcagatatcctccgcgtgaacaccgagatcacgaaggctccactctctgcctccatgatcaagcgctacgacgagcaccatcaggatctgaccctcctgaaggcgctggtccgccaacagctcccggagaagtacaaggagattttcttcgatcagtcgaagaacggctacgctgggtacatcgacggcggggcctcacaagaggagttctacaagttcatcaagccaatcctggagaagatggacggcacggaggagctcctggtgaagctcaacagggaggacctcctgcggaagcagagaaccttcgataacggcagcatcccccaccaaatccatctcggggagctgcacgccatcctgagaaggcaagaggacttctaccctttcctcaaggataaccgggagaagatcgagaagatcctgaccttcagaatcccatactacgtcggccctctcgcgcgggggaactcaagattcgcttggatgacccgcaagtctgaggagaccatcacgccgtggaacttcgaggaggtggtggacaagggcgctagcgctcagtcgttcatcgagaggatgaccaacttcgacaagaacctgcccaacgagaaggtgctccctaagcactcgctcctgtacgagtacttcaccgtctacaacgagctcacgaaggtgaagtacgtcaccgagggcatgcgcaagccagcgttcctgtccggggagcagaagaaggctatcgtggacctcctgttcaagaccaaccggaaggtcacggttaagcaactcaaggaggactacttcaagaagatcgagtgcttcgattcggtcgagatcagcggcgttgaggaccgcttcaacgccagcctcgggacctaccacgatctcctgaagatcatcaaggataaggacttcctggacaacgaggagaacgaggatatcctggaggacatcgtgctgaccctcacgctgttcgaggacagggagatgatcgaggagcgcctgaagacgtacgcccatctcttcgatgacaaggtcatgaagcaactcaagcgccggagatacaccggctgggggaggctgtcccgcaagctcatcaacggcatccgggacaagcagtccgggaagaccatcctcgacttcctcaagagcgatggcttcgccaacaggaacttcatgcaactgatccacgatgacagcctcaccttcaaggaggatatccaaaaggctcaagtgagcggccagggggactcgctgcacgagcatatcgcgaacctcgctggctcccccgcgatcaagaagggcatcctccagaccgtgaaggttgtggacgagctcgtgaaggtcatgggccggcacaagcctgagaacatcgtcatcgagatggccagagagaaccaaaccacgcagaaggggcaaaagaactctagggagcgcatgaagcgcatcgaggagggcatcaaggagctggggtcccaaatcctcaaggagcacccagtggagaacacccaactgcagaacgagaagctctacctgtactacctccagaacggcagggatatgtacgtggaccaagagctggatatcaaccgcctcagcgattacgacgtcgatcatatcgttccccagtctttcctgaaggatgactccatcgacaacaaggtcctcaccaggtcggacaagaaccgcggcaagtcagataacgttccatctgaggaggtcgttaagaagatgaagaactactggaggcagctcctgaacgccaagctgatcacgcaaaggaagttcgacaacctcaccaaggctgagagaggcgggctctcagagctggacaaggccggcttcatcaagcggcagctggtcgagaccagacaaatcacgaagcacgttgcgcaaatcctcgactctcggatgaacacgaagtacgatgagaacgacaagctgatcagggaggttaaggtgatcaccctgaagtctaagctcgtctccgacttcaggaaggatttccagttctacaaggttcgcgagatcaacaactaccaccatgcccatgacgcttacctcaacgctgtggtcggcaccgctctgatcaagaagtacccaaagctggagtccgagttcgtgtacggggactacaaggtttacgatgtgcgcaagatgatcgccaagtcggagcaagagatcggcaaggctaccgccaagtacttcttctactcaaacatcatgaacttcttcaagaccgagatcacgctggccaacggcgagatccggaagagaccgctcatcgagaccaacggcgagacgggggagatcgtgtgggacaagggcagggatttcgcgaccgtccgcaaggttctctccatgccccaggtgaacatcgtcaagaagaccgaggtccaaacgggcgggttctcaaaggagtctatcctgcctaagcggaacagcgacaagctcatcgccagaaagaaggactgggacccaaagaagtacggcgggttcgacagccctaccgtggcctactcggtcctggttgtggcgaaggttgagaagggcaagtccaagaagctcaagagcgtgaaggagctcctggggatcaccatcatggagaggtccagcttcgagaagaacccaatcgacttcctggaggccaagggctacaaggaggtgaagaaggacctgatcatcaagctcccgaagtactctctcttcgagctggagaacggcaggaagagaatgctggcttccgctggcgagctccagaaggggaacgagctcgcgctgccaagcaagtacgtgaacttcctctacctggcttcccactacgagaagctcaagggcagcccggaggacaacgagcaaaagcagctgttcgtcgagcagcacaagcattacctcgacgagatcatcgagcaaatctccgagttcagcaagcgcgtgatcctcgccgacgcgaacctggataaggtcctctccgcctacaacaagcaccgggacaagcccatcagagagcaagcggagaacatcatccatctcttcaccctgacgaacctcggcgctcctgctgctttcaagtacttcgacaccacgatcgatcggaagagatacacctccacgaaggaggtcctggacgcgaccctcatccaccagtcgatcaccggcctgtacgagacgaggatcgacctctcacaactcggcggggataagagacccgcagcaaccaagaaggcagggcaagcaaagaagaagaagtgactcgagtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgcccgggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggaggacgaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgaccggtttataaactcgcttgctgcatcagacttggcccggacttcgagccaatgttttagagctagaaatagcaagttaaaataaggctagtccgttatcaacttgaaaaagtggcaccgagtcggtgctttttttccggggcggcagggagagttttaacattgactagcgtgctgataatttgtgagaaataataattgacaagtagatactgacatttgagaagagcttctgaactgttattagtaacaaaaatggaaagctgatgcacggaaaaaggaaagaaaaagccatacttttttttaggtaggaaaagaaaaagccatacgagactgatgtctctcagatgggccgggatctgtctatctagcaggcagcagccctaccaacctcacgggccagcaattacgagtccttctaaaacgtcccgccgagggcgcgtggccgtgctgtgcagcagcacgtctaacattagtcccacctcgccagtttacagggagcagaaccagcttataagcggaggcgcggcaccaagaaggcgacttcttcgaggacagcgttttagagctagaaatagcaagttaaaataaggctagtccgttatcaacttgaaaaagtggcaccgagtcggtgcttttttt

SEQ ID NO: 40 - DNA, 7866 bp, synthetic construct
  source 1..7866: mol_type = other DNA
tgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttcctgcagggcgatctattcgaatggacaagaagtactcgatcggcctcgacatcgggacgaactcagttggctgggccgtgatcaccgacgagtacaaggtgccctctaagaagttcaaggtcctggggaacaccgaccgccattccatcaagaagaacctcatcggcgctctcctgttcgacagcggggagaccgctgaggctacgaggctcaagagaaccgctaggcgccggtacacgagaaggaagaacaggatctgctacctccaagagattttctccaacgagatggccaaggttgacgattcattcttccaccgcctggaggagtctttcctcgtggaggaggataagaagcacgagcggcatcccatcttcggcaacatcgtggacgaggttgcctaccacgagaagtaccctacgatctaccatctgcggaagaagctcgtggactccaccgataaggcggacctcagactgatctacctcgctctggcccacatgatcaagttccgcggccatttcctgatcgagggggatctcaacccagacaacagcgatgttgacaagctgttcatccaactcgtgcagacctacaaccaactcttcgaggagaacccgatcaacgcctctggcgtggacgcgaaggctatcctgtccgcgaggctctcgaagtccaggaggctggagaacctgatcgctcagctcccaggcgagaagaagaacggcctgttcgggaacctcatcgctctcagcctggggctcaccccgaacttcaagtcgaacttcgatctcgctgaggacgccaagctgcaactctccaaggacacctacgacgatgacctcgataacctcctggcccagatcggcgatcaatacgcggacctgttcctcgctgccaagaacctgtcggacgccatcctcctgtcagatatcctccgcgtgaacaccgagatcacgaaggctccactctctgcctccatgatcaagcgctacgacgagcaccatcaggatctgaccctcctgaaggcgctggtccgccaacagctcccggagaagtacaaggagattttcttcgatcagtcgaagaacggctacgctgggtacatcgacggcggggcctcacaagaggagttctacaagttcatcaagccaatcctggagaagatggacggcacggaggagctcctggtgaagctcaacagggaggacctcctgcggaagcagagaaccttcgataacggcagcatcccccaccaaatccatctcggggagctgcacgccatcctgagaaggcaagaggacttctaccctttcctcaaggataaccgggagaagatcgagaagatcctgaccttcagaatcccatactacgtcggccctctcgcgcgggggaactcaagattcgcttggatgacccgcaagtctgaggagaccatcacgccgtggaacttcgaggaggtggtggacaagggcgctagcgctcagtcgttcatcgagaggatgaccaacttcgacaagaacctgcccaacgagaaggtgctccctaagcactcgctcctgtacgagtacttcaccgtctacaacgagctcacgaaggtgaagtacgtcaccgagggcatgcgcaagccagcgttcctgtccggggagcagaagaaggctatcgtggacctcctgttcaagaccaaccggaaggtcacggttaagcaactcaaggaggactacttcaagaagatcgagtgcttcgattcggtcgagatcagcggcgttgaggaccgcttcaacgccagcctcgggacctaccacgatctcctgaagatcatcaaggataaggacttcctggacaacgaggagaacgaggatatcctggaggacatcgtgctgaccctcacgctgttcgaggacagggagatgatcgaggagcgcctgaagacgtacgcccatctcttcgatgacaaggtcatgaagcaactcaagcgccggagatacaccggctgggggaggctgtcccgcaagctcatcaacggcatccgggacaagcagtccgggaagaccatcctcgacttcctcaagagcgatggcttcgccaacaggaacttcatgcaactgatccacgatgacagcctcaccttcaaggaggatatccaaaaggctcaagtgagcggccagggggactcgctgcacgagcatatcgcgaacctcgctggctcccccgcgatcaagaagggcatcctccagaccgtgaaggttgtggacgagctcgtgaaggtcatgggccggcacaagcctgagaacatcgtcatcgagatggccagagagaaccaaaccacgcagaaggggcaaaagaactctagggagcgcatgaagcgcatcgaggagggcatcaaggagctggggtcccaaatcctcaaggagcacccagtggagaacacccaactgcagaacgagaagctctacctgtactacctccagaacggcagggatatgtacgtggaccaagagctggatatcaaccgcctcagcgattacgacgtcgatcatatcgttccccagtctttcctgaaggatgactccatcgacaacaaggtcctcaccaggtcggacaagaaccgcggcaagtcagataacgttccatctgaggaggtcgttaagaagatgaagaactactggaggcagctcctgaacgccaagctgatcacgcaaaggaagttcgacaacctcaccaaggctgagagaggcgggctctcagagctggacaaggccggcttcatcaagcggcagctggtcgagaccagacaaatcacgaagcacgttgcgcaaatcctcgactctcggatgaacacgaagtacgatgagaacgacaagctgatcagggaggttaaggtgatcaccctgaagtctaagctcgtctccgacttcaggaaggatttccagttctacaaggttcgcgagatcaacaactaccaccatgcccatgacgcttacctcaacgctgtggtcggcaccgctctgatcaagaagtacccaaagctggagtccgagttcgtgtacggggactacaaggtttacgatgtgcgcaagatgatcgccaagtcggagcaagagatcggcaaggctaccgccaagtacttcttctactcaaacatcatgaacttcttcaagaccgagatcacgctggccaacggcgagatccggaagagaccgctcatcgagaccaacggcgagacgggggagatcgtgtgggacaagggcagggatttcgcgaccgtccgcaaggttctctccatgccccaggtgaacatcgtcaagaagaccgaggtccaaacgggcgggttctcaaaggagtctatcctgcctaagcggaacagcgacaagctcatcgccagaaagaaggactgggacccaaagaagtacggcgggttcgacagccctaccgtggcctactcggtcctggttgtggcgaaggttgagaagggcaagtccaagaagctcaagagcgtgaaggagctcctggggatcaccatcatggagaggtccagcttcgagaagaacccaatcgacttcctggaggccaagggctacaaggaggtgaagaaggacctgatcatcaagctcccgaagtactctctcttcgagctggagaacggcaggaagagaatgctggcttccgctggcgagctccagaaggggaacgagctcgcgctgccaagcaagtacgtgaacttcctctacctggcttcccactacgagaagctcaagggcagcccggaggacaacgagcaaaagcagctgttcgtcgagcagcacaagcattacctcgacgagatcatcgagcaaatctccgagttcagcaagcgcgtgatcctcgccgacgcgaacctggataaggtcctctccgcctacaacaagcaccgggacaagcccatcagagagcaagcggagaacatcatccatctcttcaccctgacgaacctcggcgctcctgctgctttcaagtacttcgacaccacgatcgatcggaagagatacacctccacgaaggaggtcctggacgcgaccctcatccaccagtcgatcaccggcctgtacgagacgaggatcgacctctcacaactcggcggggataagagacccgcagcaaccaagaaggcagggcaagcaaagaagaagaagtgactcgagtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgcccgggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggaggacgaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgaccggtttataaactcgcttgctgcatcagacttgtcgtgcgcccgatcatgaacgttttagagctagaaatagcaagttaaaataaggctagtccgttatcaacttgaaaaagtggcaccgagtcggtgctttttttccggggcggcagggagagttttaacattgactagcgtgctgataatttgtgagaaataataattgacaagtagatactgacatttgagaagagcttctgaactgttattagtaacaaaaatggaaagctgatgcacggaaaaaggaaagaaaaagccatacttttttttaggtaggaaaagaaaaagccatacgagactgatgtctctcagatgggccgggatctgtctatctagcaggcagcagccctaccaacctcacgggccagcaattacgagtccttctaaaacgtcccgccgagggcgcgtggccgtgctgtgcagcagcacgtctaacattagtcccacctcgccagtttacagggagcagaaccagcttataagcggaggcgcggcaccaagaaggcctggaatatggtgtgtcacgttttagagctagaaatagcaagttaaaataaggctagtccgttatcaacttgaaaaagtggcaccgagtcggtgcttttttt